(19) United States
(12) Patent Application Publication
(10) Pub. No.: US 2006/0160879 A1
(43) Pub. Date: Jul. 20, 2006

(54) Indoles useful in the treatment of inflammation

(76) Inventors: Kristofer Olofsson, Stockholm (SE); Edgars Suna, Riga (LV); Benjamin Pelcman, Stockholm (SE); Vita Ozola, riga (LV); Martins Katkevics, Riga (LV); Ivars Kalvins, Riga (LV); Wesley Schaal, Stockholm (SE)

Correspondence Address: NIXON & VANDERHYE, PC, 901 NORTH GLEBE ROAD, 11TH FLOOR, ARLINGTON, VA, 22203, US

(21) Appl. No.: 10/563,464

(22) PCT Filed: Jul. 9, 2004
(86) PCT No.: PCT/GB04/02996

§ 371 Date: Feb. 14, 2006

Related U.S. Application Data
(60) Provisional application No. 60/485,390, filed on Jul. 9, 2003.

(30) Foreign Application Priority Data
Jul. 9, 2003 (SE) 0302035-1

(51) Int. Cl. A61K 31/405; C07D 403/02; C07D 209/18

(52) U.S. Cl. 514/414; 514/419; 548/465; 548/495

(57) ABSTRACT

There is provided a compound of formula (I), wherein X, Y, Z, R¹, R², R³, R⁴, and R⁵ have meanings given in the description, and pharmaceutically-acceptable salts thereof, which compounds are useful in the treatment of diseases in which inhibition of the activity of microsomal prostaglandin E synthase-1 is desired and/or required, and particularly in the treatment of inflammation.

### FIELD OF THE INVENTION

This invention relates to novel pharmaceutically-useful compounds, which compounds are useful as inhibitors of microsomal prostaglandin E synthase-1 (mPGES-1). The compounds are of potential utility in the treatment of inflammatory diseases. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several malignancies are known to have inflammatory components adding to the symptomatology of the patients.

The cyclooxygenase (COX) enzyme exists in two forms, one that is constitutively expressed in many cells and tissues (COX-1), and one that is induced by pro-inflammatory stimuli, such as cytokines during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites of arachidonic acid, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic.

In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

mPGES-1 belongs to the membrane-associated proteins in the eicosanoid and glutathione metabolism (MAPEG) family. Other members of this family include the 5-lipoxygenase-activating protein (FLAP), leukotriene C₄ synthase and microsomal glutathione S-transferases (MGST1, MGST2 and MGST3).

Thus, agents that are capable of inhibiting the action of mPGESs and, in particular, mPGES-1, and thus reducing the formation of the specific arachidonic acid metabolite PGE₂, are likely to be of benefit in the treatment of inflammation.

### PRIOR ART

Various indoles, and derivatives thereof, have been disclosed in international patent applications WO 01/30343, WO 96/03377 and WO 99/33800, U.S. Pat. Nos. 5,189,054 and 6,525,083 and European patent application EP 483 881. However, none of these documents disclose or suggest the use of the compounds disclosed therein in the treatment of inflammation.

Structurally related indoles have been disclosed for potential use in the treatment of inflammation in international patent application WO 99/05104 and European patent application EP 985 666. However these documents do not disclose or suggest indoles that are substituted in the 1-position.

Other related indoles have been disclosed for potential use in the treatment of inflammation in international patent application WO 94/13662, U.S. Pat. No. 5,081,145 and European patent application EP 535 924. However, none of these documents disclose or suggest indoles that are directly substituted at the benzenoid moiety of the indole ring with an aryl or heteroaryl group.

U.S. Pat. No. 5,081,138 and European patent application EP 166 591 disclose indoles for potential use in the treatment of inflammation. However, neither of these documents disclose or suggest indoles that are directly substituted at the 2-position of the indole ring with a carboxylic acid group or a derivative thereof.

International patent applications WO 99/07351 and WO 99/07678 disclose indoles for potential use in the treatment of inflammation but do not disclose or suggest indoles that are substituted in the indolic 3-position by an aryl, a heteroaryl or an amide group.

International patent applications WO 00/46198, WO 00/46197, WO 00/46195, WO 00/46199, WO 94/14434, WO 96/18393 and WO 02/30895 describe indole compounds for potential use in the treatment of inflammation. However, there is no specific disclosure in any of these documents of substitution at the benzenoid moiety of the indole ring with an aryl or heteroaryl group.

International patent applications WO 98/08818 and WO 99/43672 also disclose indoles for potential use in the treatment of inflammation. However, there is no specific disclosure in either of these documents of indoles substituted in the 3-position by an aryl or heteroaryl group, or a nitrogen derivative, such as an amine, amide or sulfonamide group attached to the indole ring through the nitrogen atom.

Finally, international patent applications WO 99/43654 and WO 99/43651 describe indole compounds for potential use in the treatment of inflammation. These documents do not specifically disclose indoles substituted in the 3-position by an aryl or heteroaryl group, or a nitrogen derivative, such as an amine, amide or sulfonamide group attached to the indole ring through the nitrogen atom, and which are further substituted at is the benzenoid moiety of the indole ring with an aryl or heteroaryl group.

### DISCLOSURE OF THE INVENTION

According to the invention there is provided a compound of formula I, 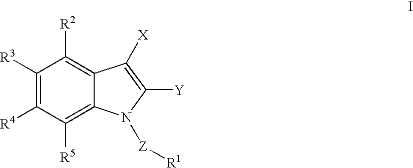
wherein
X represents:
i) an aryl group or a heteroaryl group, both of which groups are optionally substituted by one or more substituents selected from A; or
ii) —N(R⁶)-E-R⁷;
E represents a single bond, —C(O)— or —S(O)ₙ—;
Y represents —CH₂OH, —C(O)N(H)R⁸, —C(O)N(H)OR⁸ or —(O)OR⁸;
Z represents a C₁₋₈ alkylene or a C₂₋₈ heteroalkylene chain, both of which:
(i) optionally contain one or more unsaturations (for example double or triple bonds);
(ii) are optionally substituted by one or more substituents selected from halo, —R⁸, —N(R⁸)(R⁹), —OR and ═O; and/or
(iii) may form part of an additional 3- to 8-membered ring formed is between any one or more (e.g. one or two) members of the C₁₋₈ alkylene or C₂₋₈ heteroalkylene chain, which ring optionally contains 1 to 3 heteroatoms and/or 1 to 3 unsaturations (for example double or triple bonds) and which ring is itself optionally substituted by one or more substituents selected from halo, —R⁸, —N(R⁸)(R⁹), —OR⁸ and ═O;
R¹ represents an aryl or a heteroaryl group, both of which are optionally substituted by one or more substituents selected from A;
one of the groups R², R³, R⁴ and R⁵ represents an aryl group or a heteroaryl group (both of which are optionally substituted by one or more substituents selected from A) and:
a) the other groups are independently selected from hydrogen, G¹, an aryl group, a heteroaryl group (which latter two groups are optionally substituted by one or more substituents selected from A), C₁₋₆ alkyl, C₃₋₁₀ (e.g. C₃₋₈) cycloalkyl, C₂₋₆ alkenyl, C₂₋₆ alkynyl or C₃₋₈ heterocycloalkyl (which latter five groups are optionally substituted by one or more substituents selected from G¹ and/or Q¹); and/or
b) any two other groups which are adjacent to each other are optionally linked to form, along with two atoms of the essential benzene ring in the compound of formula I, a 5- to 6-membered ring, optionally containing 1 or more (e.g. 1 to 3) heteroatoms and/or 1 to 3 unsaturations (for example double or triple bonds), which ring is itself optionally substituted by one or more substituents selected from halo, —R⁸, —OR⁸ and ═O;
A represents, on each occasion when mentioned above:
I) an aryl group or a heteroaryl group, both of which are optionally substituted by one or more substituents selected from B;
II) a C₁₋₆ alkyl, C₃₋₁₀ (e.g. C₃₋₈) cycloalkyl, C₂₋₆ alkenyl, C₂₋₆ alkyl or C₃₋₈ heterocycloalkyl group, all of which are optionally substituted by one or more substituents selected from G¹ and/or Q¹; or
III) a G¹ group; or
IV) two adjacent A substituents may be linked together to form, along with the essential atoms of the aryl or heteroaryl group to which the two A substituents are attached, a further 5- to 6-membered ring, optionally containing 1 or more (e.g. 1 to 3) heteroatoms and/or 1 to 3 unsaturations (for example double or triple bonds), which ring is itself optionally substituted by one or more substituents selected from halo, —R⁸, —OR⁸ and ═O; G¹ represents, on each occasion when mentioned above, halo, cyano, —N₃, —NO₂, —ONO₂ or -A¹-R¹⁰;
wherein A¹ represents a single bond or a spacer group selected from —C(Q²)A²-, —S(O)ₙA³-, —N(R¹¹)A⁴-, —OA⁵- and —S—, in which:
A² represents A⁶ or —S—;
A³ represents A⁶;
A⁴ represents A⁷, —C(Q²)N(R¹¹)C(Q²)N(R¹¹)—, —C(Q²)N(R¹¹)C(Q²)O—, —C(Q²)N(R¹¹)S(O)ₙN(R¹¹)—, —C(Q²)S—, —S(O)ₙN(R¹¹)C(Q²)N(R¹¹)—, —S(O)ₙN(R¹¹)C(Q²)O—, —S(O)ₙN(R¹¹)S(O)ₙN(R¹¹)— or —S(O)ₙO—;
A⁵ represents A⁷ or —S(O)ₙO—;
A⁶ represents a single bond, —N(R¹¹)— or —O—;
A⁷ represents a single bond, —C(Q²)-, —C(Q²)N(R¹¹)—, —C(Q²)O—, —S(O)ₙ—, or —S(O)ₙN(R¹¹);
Q¹ and Q² independently represent, on each occasion when mentioned above, ═O, ═S, ═NR¹⁰, ═NN(R¹⁰)(R¹¹), ═NOR¹⁰, ═NS(O)₂N(R¹⁰)(R¹¹), ═NCN, ═C(H)NO₂ or ═C(R¹⁰)(R¹¹);
R⁶ and R⁷ independently represent, on each occasion when mentioned above:
I) hydrogen;
II) an aryl group or a heteroaryl group, both of which are optionally substituted by one or more substituents selected from B; or
III) a C₁₋₆ allyl, C₃₋₁₀ (e.g. C₃₋₈) cycloalkyl, C₂₋₆ alkenyl, C₂₋₆ alkynyl or C₃₋₈ heterocycloalkyl group, all of which groups are optionally substituted by one or more substituents selected from G² and/or Q³; or
R⁶ and R⁷ may be linked together to form along with the N atom and -E- group to which R⁶ and R⁷ are respectively attached, a 5- to 8-membered ring, optionally containing 1 to 3 heteroatoms and/or 1 to 3 unsaturations (for example double or triple bonds), which ring is optionally substituted by one or more substituents selected from G² and/or Q³;
B represents, on each occasion when mentioned above:
I) an aryl group or a heteroaryl group, both of which are optionally substituted by one or more substituents selected from G² and/or wherein any two adjacent atoms of the aryl or heteroaryl group may be linked together to form a further 5- to 6-membered ring, optionally containing 1 or more (e.g. 1 to 3) heteroatoms and/or 1 to 3 unsaturations (for example double or triple bonds), which ring is itself optionally substituted by one or more substituents selected from halo, —R⁸, —OR⁸ and ═O;
II) a C₁₋₆ alkyl, C₃₋₁₀ (e.g. C₃₋₈) cycloalkyl, C₂₋₆ alkenyl, C₂₋₆ alkynyl or C₃₋₈ heterocycloalkyl group, all of which are optionally substituted by one or more substituents selected from G² and/or Q³; or
III) a G² group; or
IV) two adjacent B substituents may be linked together to form, along with the essential atoms of the aryl or heteroaryl group to which the two B substituents are attached, a further 5- to 6-membered ring, optionally containing 1 or more (e.g. 1 to 3) heteroatoms and/or 1 to 3 unsaturations (e.g. double or triple bonds), which ring is itself optionally substituted by one or more substituents selected from halo, —R⁸, —OR⁸ and ═O;
G² represents, on each occasion when mentioned above, halo, cyano, —N₃, —NO₂, —ONO₂ or -A⁸-R¹²;
wherein A⁸ represents a single bond or a spacer group selected from —C(Q⁴)A⁹-, —S(O)ₙA¹⁰-, —N(R¹³)A¹¹-, —OA¹²- and —S—, in which:
A⁹ represents A¹³ or —S—;
A¹⁰ represents A¹³;
A¹¹ represents A¹⁴, C(Q⁴)N(R¹³)C(Q⁴)N(R¹³)—, —C(Q⁴)N³)C(Q⁴)O—, —C(Q⁴)N(R¹³)S(O)ₙN(R¹³)—, —C(Q⁴)S—, —S(O)ₙNR¹³)C(Q⁴)N(R¹³)—, —S(O)ₙN(R¹³)C(Q⁴)O—, —S(O)ₙN(R¹³)S(O)ₙN(R¹³)— or —S(O)ₙO—;
A¹² represents A¹⁴ or —S(O)ₙO—;
A¹³ represents a single bond, —N(R¹³)— or —O—;
A¹⁴ represents a single bond, —C(Q⁴)-, —C(Q⁴)N(R³)—, —C(Q⁴)O—, —S(O)ₙ— or
Q³ and Q⁴ independently represent, on each occasion when mentioned above, ═O, S, ═NR¹², ═NN(R¹²)(R¹³), ═NOR¹², ═NS(O)₂N(R¹²)(R³), ═NCN, ═C(H)NO₂ or ═C(R¹²)(R¹³);
R⁸, R⁹, R¹⁰, R¹¹, R¹² and R¹³ are independently selected from:
i) hydrogen;
ii) an aryl or a heteroaryl group, both of which are optionally substituted by one or more substituents selected from G³ and/or wherein any two adjacent atoms of the aryl or heteroaryl group may be linked together to form a further 5- to 6-membered ring, optionally containing 1 or more (e.g. 1 to 3) heteroatoms, which ring is itself optionally substituted by one or more substituents selected from halo, —R¹⁴, —OR¹⁴ and ═O; or
iii) a C₁₋₆ alkyl, C₃₋₁₀ (e.g. C₃₋₈) cycloalkyl, C₂₋₆ alkenyl, C₂₋₆ alkynyl or C₃₋₈ heterocycloalkyl group, all of which are optionally substituted by one or more substituents selected from G³ and/or W¹; or
any pair of R⁸, R⁹, R¹⁰, R¹¹, R¹² and R¹³ may, for example when present on the same or on adjacent atoms, be linked together to form with those, or other relevant, atoms, a further 5- to 8-membered ring, optionally containing 1 to 3 heteroatoms and/or 1 to 3 unsaturations (for example double or triple bonds), which ring is itself optionally substituted by one or more substituents selected from G³ and/or W¹;
G³ represents, on each occasion when mentioned above, halo, cyano, —N₃, —NO₂, —ONO₂ or -A¹⁵-R¹⁵;
wherein A¹⁵ represents a single bond or a spacer group selected from —C(W²)A⁶-, —S(O)ₙA¹⁷-, —N(R⁶)A¹⁸-, —OA¹⁹- and —S—, in which:
A¹⁶ represents A²⁰ or —S—;
A¹⁷ represents A²⁰;
A¹⁸ represents A²¹, —C(W²)N(R¹⁶)C(O)C(W²)O—, —C(W²)N(R¹⁶)S(O)ₙN(R¹⁶)—, —C(W²)S—, —S(O)ₙN(R¹⁶)C(W²)N(R¹⁶)—, —S(O)ₙN(R¹⁶)C(W²)O—, —S(O)ₙN(R¹⁶)S(O)ₙN(R¹⁶)— or —S(O)ₙO—;
A¹⁹ represents A²¹ or —S(O)ₙO—;
A²⁰ represents a single bond, —N(R¹⁶)— or —O—;
A²¹ represents a single bond, —C(W²)—, —C(W²)N(R¹⁶)—, —C(W²)O—, —S(O)ₙ— or —S(O)ₙN(R¹⁶);
W¹ and W² independently represent, on each occasion when mentioned above, ═O, ═S, ═NR¹⁵, ═NN(R¹⁵)(R¹¹⁶), ═NOR¹⁵, ═NS(O)₂N(R¹⁵)(R¹⁶), ═NCN, ═C(H)NO₂ or ═C(R¹⁵)(R¹⁶);
R¹⁴, R¹⁵ and R¹⁶ are independently selected from:
i) hydrogen;
ii) an aryl or a heteroaryl group, both of which are optionally substituted by one or more substituents selected from G⁴, methylenedioxy, difluoromethylenedioxy and/or dimethylmethylenedioxy; or
iii) a C₁₋₆ alkyl, C₃₋₁₀ (e.g. C₃₋₈) cycloalkyl, C₂₋₆ alkenyl, C₂₋₆ alkynyl or C₃₋₈ heterocycloalkyl group, all of which are optionally substituted by one or more substituents selected from G⁴ and/or J; or
any pair of R¹⁴, R¹⁵ and R¹⁶ may, for example when present on the same or on adjacent atoms, be linked together to form with those, or other relevant, atoms, a further 5- to 7-membered ring, optionally containing 1 to 3 heteroatoms and/or 1 to 3 unsaturations (for example double or triple bonds), which ring is itself optionally substituted by one or more substituents selected from G⁴ and J;
G⁴ represents, on each occasion when mentioned above, halo, cyano, —N₃, —NO₂, —ONO₂ or -A²²-R¹⁷;
wherein A²² represents a single bond or a spacer group selected from C(O)A²³-, S(O)ₙA²⁴-, —N(R¹⁸)A²⁵-, —OA²⁶- and —S—, in which:
A²³ represents A²⁷ or —S—;
A²⁴ represents A²⁷;
A²⁵ represents A²⁸, C(O)N(R¹⁸)C(O)N(R¹⁸)_, C(O)N(R¹⁸)C(O)O—, —C(O)N(R¹⁸)S(O)ₙN(R¹⁸)—, —C(O)S—, —S(O)ₙN(R¹⁸)C(O)N(R¹⁸)—, —S(O)ₙN(R¹⁸)C(O)O—, —S(O)ₙN(R¹⁸)S(O)ₙN(R¹⁸)— or —S(O)ₙO—;
A²⁶ represents A²⁸ or —S(O)ₙO—;
A²⁷ represents a single bond, —N(R¹⁸)— or —O—;
A²⁸ represents a single bond, —C(O)—, —C(O)N(R¹⁸)—, —C(O)O—, —S(O)ₙ— or —S(O)ₙN(R¹⁸);
J represents, on each occasion when mentioned above, ═O, ═S, ═NR¹⁷, ═NN(R¹⁷)(R¹⁸), ═NOR¹⁷, ═NS(O)₂N(R¹⁷)(R¹⁸), ═NCN, ═C(H)NO₂ or ═C(R¹⁷)(R¹⁸);
R¹⁷ and R¹⁸ are independently selected from hydrogen and C₁₋₆ alkyl, which latter group is optionally substituted by one or more substituents selected from halo, —NH₂, —N(H)Me, —N(H)Et, —N(H)i-Pr, —NMe₂, —N(Me)Et, —N(Me)i-Pr, —NEt₂, —OH, —OMe, —OEt, -Oi-Pr and ═O; and
n represents, on each occasion when mentioned above, 1 or 2,
or a pharmaceutically-acceptable salt thereof,
which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as HPLC or chromatography over silica, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of three) of carbon atoms, be branched-chain. C₁₋₆ alkyl groups that may be mentioned include methyl, ethyl, n-propyl, isopropyl, n-butyl, sec-butyl, isobutyl, tert-butyl, n-pentyl, isopentyl, 77-hexyl, and isohexyl.

C_{1-q} alkylene chains (where q is the upper limit of the range) defined herein are straight-chain alkyl groups, which groups are attached at each terminal end.

C_{v-q} heteroalkylene chains (where v is the lower limit and q is the upper limit of the range) defined herein are C_{v-q} alkylene chains, wherein one or more (e.g. 1 to q−1) carbon atoms have been replaced with a heteroatom.

C_{2-q} alkenyl groups (where q is the upper limit of the range) defined herein may be straight-chain or, when there is a sufficient number (i.e. a minimum of three) of carbon atoms, be branched-chain. Such alkenyl groups may contain one or more double bonds. C₂₋₆ alkenyl groups that may be mentioned include ethenyl (i.e. vinyl), 1-propenyl, 2-propenyl (i.e. allyl), propadienyl, 1-butenyl, 2-butenyl, 3-butenyl, 1-butadienyl, 1-pentenyl, 2-pentenyl, 4-pentenyl, and 5-hexenyl.

C_{2-q} alkynyl groups (where q is the upper limit of the range) defined herein may be straight-chain or, when there is a sufficient number (i.e. a minimum of four) of carbon atoms, be branched-chain. Such alkynyl groups may contain one or more triple bonds. C₂₋₆ alkynyl groups that may be mentioned include ethynyl, 1-propynyl, 2-propynyl, 1-butynyl, 2-butynyl, 3-butynyl, 1-pentynyl, 2-pentynyl, 4-pentynyl, 1-hexynyl, 3-hexynyl, and 5-hexynyl.

C_{3-q} cycloalkyl groups (where q is the upper limit of the range) that may be mentioned include monocyclic or bicyclic alkyl groups, or fused ring systems such as three fused cycloalkyl groups. Such cycloalkyl groups may be saturated or unsaturated containing one or more double or triple bond (forming for example a C_{3-q} cycloalkenyl or a C_{3-q} cycloalkynyl group). C_{3-q} (e.g. C₃₋₁₀) cycloalkyl groups that may be mentioned include adamantyl, cyclopropyl, cyclobutyl, cyclopentyl, cyclohexyl, cycloheptyl, cyclooctyl, cyclopentenyl, cyclohexenyl, cycloheptenyl, cyclooctenyl, cyclooctynyl, bicycloheptyl, bicyclooctyl, and bicyclooctenyl. Substituents may be attached at any point on the cycloalkyl group. Further in the case where the substituent is another cyclic compound, then the cyclic substituent may be attached through a single atom on the cycloalkyl group, forming a so-called “spiro”-compound.

C_{3-q} heterocycloalkyl groups (where q is the upper limit of the range) that may be mentioned include monocyclic or bicyclic alkyl groups in which at least one (e.g. one to four) of the atoms in the ring system is other than carbon (i.e. a hetereoatom). Further, such heterocycloalkyl groups may be saturated or unsaturated containing one or more double and/or triple bonds, forming for example a C_{3-q} heterocycloalkenyl or a C_{3-q} heterocycloalkynyl group. C_{3-q} heterocycloalkyl groups that may be mentioned include aziridinyl, azetidinyl, dihydropyranyl, dihydropyridyl, dihydropyrrolyl (including 2,5-dihydropyrrolyl), dioxolanyl (including 1-dioxolanyl), dioxanyl (including 1-dioxanyl and 1,4-dioxanyl), dithianyl (including 1,4-dithianyl), dithiolanyl (including 1-dithiolanyl), imidazolidinyl, imidazolinyl, morpholinyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl, thietanyl, thiiranyl, thiolanyl, thiomorpholinyl, trithianyl (including 1,5-trithianyl), tropanyl and the like. Substituents on the heterocycloalkyl groups may, where appropriate, be located on any atom in the ring system including a heteroatom. Further in the case where the substituent is another cyclic compound, then the cyclic substituent may be attached through a single atom on the heterocycloalkyl group, forming a so-called “spiro”-compound. The point of attachment of a heterocycloalkyl group may be via any atom in the ring system including (where appropriate) a heteroatom, or an atom on any fused carbocyclic ring that may be present as part of the ring system. Heterocycloalkyl groups may also be in the N- or S-oxidised form.

The term “halo”, when used herein, includes fluoro, chloro, bromo and iodo.

Aryl groups that may be mentioned include C₆₋₁₃ aryl (e.g. C₆₋₁₀) groups. Such groups may be monocyclic, bicyclic or tricylic and have between 6 and 13 ring carbon atoms, in which at least one ring is aromatic. C₆₋₁₃ aryl groups include phenyl, naphthyl and the like, such as 1,2,3,4-tetrahydronaphthyl, indanyl, indenyl and fluorenyl. The point of attachment of aryl groups may be via any atom of the ring system.

Heteroaryl groups that may be mentioned include those which have between 5 and 10 members. Such groups may be monocyclic, bicyclic or tricyclic, in which at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1-benzodioxolyl), benzofuranyl, benzofurazanyl, benzothiazolyl (including 2,1-benzothiazolyl), benzoxadiazolyl (including 2,1-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiazolyl, isoxazolyl, naphthyridinyl (including 1,5-naphthyridinyl and 1,8-naphthyridinyl), oxadiazolyl (including 1,2,3-oxadiazolyl, 1,2,4-oxadiazolyl and 1,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,4-thiadiazolyl), thiazolyl, thiochromanyl, thienyl, triazolyl (including 1,2,3-triazolyl, 1,2,4-triazolyl and 1,4-triazolyl) and the like. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form.

Heteroatoms that may be mentioned include oxygen, nitrogen, sulphur and selenium.

For the avoidance of doubt, optionally substituted methylenedioxy groups, when attached to a ring system, are formed between any two adjacent atoms of the ring system.

For the avoidance of doubt, in cases in which the identity of two or more substituents in a compound of the invention may be the same, the actual identities of the respective substituents are not in any way interdependent. For example, in the situation in which R¹ and R³ are both aryl groups substituted by one or more C₁₋₆ alkyl groups, the alkyl groups in question may be the same or different. Similarly, when groups are substituted by more than one substituent as defined herein, the identities of those individual substituents are not to be regarded as being interdependent. For example, when X and/or R¹ represents e.g. an aryl group substituted by G¹ in addition to, for example, C₁₋₆ alkyl, which latter group is substituted by G¹, the identities of the two G¹ groups are not to be regarded as being interdependent.

Compounds that may be mentioned include those in which:

E represents —C(O)—;

when any two adjacent R², R³, R⁴ or R⁵ groups, adjacent A substituents, adjacent B substituents, and/or, when B represents aryl or heteroaryl, adjacent atoms of those aryl or heteroaryl groups are linked to form a 5- to 6-membered ring, then that ring is fully saturated.

Preferred compounds of the invention include those in which:

Y represents —CH₂OH or, more preferably, —C(O)N(H)R⁸, —C(O)N(H)OR⁸ or —C(O)OR⁸;

A⁸ represents a single bond, —C(Q⁴)A⁹-, —S(O)ₙA¹¹-, —N(R¹³)A¹¹- or —OA¹²-;

A⁹ represents A¹³;

A¹¹ represents A¹⁴;

Q³ represents ═O, ═NN(R¹²)(R¹³), ═NOR¹², ═NS(O)₂N(R¹²)(R¹³), ═NCN or ═C(H)NO₂;

Q⁴ represents ═O;

A¹⁵ represents a single bond, —C(W²)A¹⁶-, —S(O)ₙA⁷-, —N(R¹⁶)A¹⁸- or —OA¹⁹-:

A¹⁶ represents A²⁰;

A¹⁸ represents A²¹;

W¹ represents ═O, ═NN(R¹⁵)(R¹⁶), ═NOR¹⁵, ═NS(O)₂N¹⁵)(R¹⁶), ═NCN or ═C(H)NO₂;

W² represents ═O;

when any pair of R¹⁴, R¹⁵ and R¹⁶ is linked together to form a further optionally substituted 5- to 7-membered ring, then that ring optionally contains only 1 unsaturation;

A²² represents a single bond, —C(O)A²³-, —S(O)ₙA²⁴-, —N(R¹⁸)A²⁵- or —OA²⁶-;

A²³ represents A²⁷;

A²⁵ represents A²⁸; and/or

J represents ═O, ═NN(R¹⁷)(R¹⁸), ═NOR¹⁷, ═NS(O)₂N(R⁷)(R⁸), ═NCN, or ═C(H)NO₂;

More preferred compounds of the invention include those in which:

Y represents —CH₂OH or, more preferably, —C(O)N(H)R⁸ or —C(O)OR⁸;

when any two members of the C₁₋₈ alkylene or C₂₋₈ heteroalkylene chain that Z may represent form part of an additional optionally substituted 3- to 8-membered ring, then that ring optionally contains 1 to 2 heteroatoms and/or 1 unsaturation;

A¹ represents —S— or, more preferably, a single bond, —C(Q²)A²-, —S(O)ₙA³-, —N(R¹¹)A⁴- or —OA⁵-:

A² represents A⁶;

A⁴ represents A⁷;

Q¹ represents ═O, ═NOR¹⁰, ═NS(O)₂N(R¹⁰)(R¹¹), ═NCN or ═C(H)NO₂;

Q² represents ═O;

when two adjacent atoms of the optionally substituted aryl or heteroaryl group that B may represent are linked together to form a further optionally substituted ring, then the linking divalent substituent that forms part of that ring is selected from methylenedioxy, difluoromethylenedioxy and dimethylmethylenedioxy;

Q³ represents ═O, ═NOR¹², ═NS(O)₂N(R¹²)(R¹³), ═NCN or ═C(H)NO₂;

when R⁶ and R⁷ are linked to form an optionally substituted 5- to 8-membered ring, then that ring optionally contains only 1 unsaturation;

when two adjacent atoms of the optionally substituted aryl or heteroaryl group that R⁸, R⁹, R¹⁰, R¹¹, R¹² and/or R¹³ may represent are linked together to form a further optionally substituted ring, then the linking divalent substituent that forms part of that ring is selected from methylenedioxy, difluoromethylenedioxy and dimethylmethylenedioxy;

W¹ represents ═O, ═NOR¹⁵, ═NS(O)₂N(R¹⁵)(R¹⁶), ═NCN or ═C(H)NO₂; and/or

R¹⁴, R¹⁵ and R¹⁶ independently represent an aryl or heteroaryl group, both of which groups are optionally substituted by one or more substituents selected from halo, —NH₂, —N(H)Me, —N(H)Et, —N(H)i-Pr, —NMe₂, —N(Me)Et, —N(Me)i-Pr, —NEt₂, —OH, —OMe, —OEt, -Oi-Pr and ═O or, R¹⁴, R¹⁵ and R¹⁶ are, more preferably, hydrogen or C₁₋₆ alkyl, which latter group is optionally substituted by one or more substituents selected from halo, —NH₂, —N(H)Me, —N(H)Et, —N(H)i-Pr, —NMe₂, —N(Me)Et, —N(Me)i-Pr, —NEt₂, —OH, —OMe, —OEt, -Oi-Pr and ═O;

More preferred compounds include those in which:

Y represents —CH₂OH, —C(O)NHR⁸ or, more preferably, —C(O)OR⁸;

when any one or more members of the C₁₋₈ alkylene or C₂₋₈ heteroalkylene chain that Z may represent form part of an additional ring, then that ring is a cyclopropyl group formed together with the same or adjacent carbon atoms of that C₁₋₈ alkylene or C₂₋₈ heteroalkylene chain;

when any adjacent pair of R², R³, R⁴ or R⁵ is linked, to form an optionally substituted ring, then the linking divalent substituent that forms part of that ring is a methylenedioxy, difluoromethylenedioxy or dimethylmethylenedioxy group;

when any two adjacent A substituents are linked to form an optionally substituted ring, then the linking divalent substituent that forms part of that ring is selected from methylenedioxy, difluoromethylenedioxy and dimethylmethylenedioxy;

when any two adjacent B substituents are linked to form an optionally substituted ring, then the linking divalent substituent that forms part of that ring is selected from methylenedioxy, difluoromethylenedioxy and dimethylmethylenedioxy;

when R⁶ and R⁷ are linked to form an optionally substituted ring, then that ring is a 5- to 6-membered ring;

when any pair of R⁸, R⁹, R¹⁰, R¹¹, R¹² and R¹³ is linked to form a further optionally substituted ring, then that ring optionally contains only 1 unsaturation; and/or

n represents 2;

Preferred compounds of the invention include those in which R¹, and (when they represent an aryl or a heteroaryl group) X, R², R³, R⁴ and/or R⁵ represent an optionally substituted fluorenyl, phenyl, naphthyl, pyrrolyl, furanyl, thienyl, pyrazolyl, imidazolyl, oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1-benzodioxolyl, benzothiazolyl, and/or benzodioxanyl, group

Preferred values of R¹ include optionally substituted fluorenyl (e.g. 2-fluorenyl) or pyridyl and, especially, optionally substituted phenyl.

Preferred values of X, when X represents an optionally substituted aryl or heteroaryl group include optionally substituted phenyl, thienyl (e.g. 2-thienyl), pyridyl (e.g. 3-pyridyl and 4-pyridyl), pyrazolyl, pyrazinyl or quinolinyl.

Preferred values of R², R³, R⁴ and R⁵, when they represent an optionally substituted aryl or heteroaryl group, include optionally substituted phenyl, pyridyl (e.g. 3-pyridyl) or naphthyl (e.g. l-naphthyl).

Further preferred compound of the invention include those in which:

Z represents C₁₋₆ alkylene, such as methylene, propylene or hexylene, in which one of the carbon atoms in the chain may be replaced with a heteroatom (e.g. oxygen) so forming, for example, an oxypentylene group;

R² represents hydrogen or G¹;

R³ represents hydrogen, phenyl or pyridyl (e.g. 3-pyridyl), which latter two groups are optionally substituted by one or more substituents selected from A;

R⁴ represents hydrogen, phenyl or naphthyl, which latter two groups are optionally substituted by one or more substituents selected from A;

R⁵ represents hydrogen or phenyl, which latter group is optionally substituted by one or more substituents selected from A or is preferably unsubstituted;

when R², R³, R⁴ or R⁵ represents an optionally substituted phenyl, pyridyl or naphthyl group, then the other substituents on the essential benzene ring of the indole of formula I, (i.e. R², R³, R⁴ or R⁴ (as appropriate)) represent hydrogen or G¹;

A represents G¹ or any two adjacent A substituents may be linked to form a further ring, wherein the linking divalent substituent that forms part of that ring is preferably methylenedioxy, which group is preferably unsubstituted;

G¹ represents halo (such as chloro or fluoro), cyano, —NO₂ or -A¹-R¹⁰;

A² represents A⁶;

A³ represents a single bond;

A⁴ represents A⁷;

A⁵ represents A⁷ and, preferably, a single bond;

A⁷ represents a single bond, —C(Q²)- or —S(O)₂—;

Q² represents ═O;

R⁶ represents hydrogen or C₁₋₃ alkyl group (such as methyl or ethyl), which latter group is optionally substituted by G²;

R⁷ represents an aryl group (such as phenyl) or a heteroaryl group (such as pyridyl), which latter two groups are optionally substituted by one or more substituents selected from B, or R⁷ represents C₁₋₄ alkyl (such as methyl, ethyl, propyl, butyl (e.g. n-butyl or t-butyl)), C₂₋₄ alkenyl (such as ethenyl) or C₅₋₁₀ cycloalkyl (such as cyclohexyl or adamantyl), which latter three groups are optionally substituted by one or more substituents selected from G²; or

R⁶ and R⁷ are optionally linked to form a 5- to 6-membered ring optionally substituted by ═O;

B represents G²,

G² represents halo (such as chloro or fluoro), cyano, —NO₂ or -A⁸-R¹²;

A⁸ represents a single bond, —N(R¹³)A¹¹- or —OA¹²-;

A¹¹ and A¹² independently represent A¹⁴;

A¹⁴ represents a single bond;

R⁸ represents C₁₋₃ alkyl (such as ethyl) or, preferably, hydrogen;

R¹⁰ represents hydrogen, aryl (such as phenyl), heteroaryl (such as tetrazolyl), C₁₋₄ alkyl (such as methyl, ethyl, isopropyl or butyl (e.g. n-butyl or t-butyl)), C₂₋₄ alkenyl (such as ethenyl or butenyl (e.g. but-3-enyl)) or C₅₋₆ cycloalkyl (such as cyclohexyl), which latter five groups are optionally substituted by one or more substituents selected from G³;

R¹¹ represents hydrogen or C₂₋₄ alkenyl (such as propenyl (e.g. propen-2-yl, i.e. allyl));

R¹² represents hydrogen, an aryl group (such as a phenyl group), a heteroaryl group (such as a pyrrolyl group), C₁₋₄ alkyl (such as methyl, isopropyl or butyl (e.g. n-butyl or t-butyl)) or C₅₋₁₀ cycloalkyl (such as cyclohexyl or adamantyl) which latter four groups are optionally substituted by one or more substituents selected from G³;

R¹³ represents hydrogen or C₁₋₃ alkyl (such as methyl); G³ represents halo (such as fluoro) or -A¹⁵-R¹⁵;

A¹⁵ represents a single bond or —OA¹⁹-;

A¹⁹ represents a single bond;

R¹⁵ represents hydrogen, C₁₋₃ alkyl (such as C₁₋₂ alkyl (e.g. methyl)) or aryl (such as phenyl).

Preferred optional substituents on R¹, R⁶, R⁷ and (when they represent an aryl or heteroaryl group) X, R², R³, R⁴ and R⁵ groups are selected from: halo (e.g. chloro, fluoro or bromo);

—NO₂;

cyano;

methylenedioxy;

C₁₋₆ alkyl, which alkyl group may be linear or branched (e.g. C₁₋₄ alkyl (including methyl, ethyl, n-propyl, isopropyl, n-butyl, s-butyl, isobutyl or t-butyl), n-pentyl, isopentyl, n-hexyl or isohexyl), and which alkyl groups are optionally substituted by one or more substituents selected from a halo (e.g. fluoro) group (so forming, for example, —CH₂F, —CHF₂ or —CF₃), an aryl group (such as phenyl) and OR¹⁹;

C₂₋₆ alkenyl (e.g. ethenyl, 1-propenyl, 2-propenyl, 1-butenyl, 2-butenyl, 3-butenyl, 1-pentenyl, 2-pentenyl, 4-pentenyl or 5-hexenyl);

C₃₋₁₀ (e.g. C₃₋₈) cycloalkyl (e.g. cyclopropyl, cyclobutyl, cyclopentyl, cyclohexyl or cyclooctyl), optionally substituted with C₁₋₆ alkyl (such as methyl);

phenyl, optionally substituted with one or more substituents selected from halo (e.g. fluoro or, especially, chloro) and OR¹⁹;

a heteroaryl group selected from tetrazolyl and pyrrolyl, optionally substituted by one or more C₁₋₆ alkyl group (such as methyl);

methylthio, methylsulfinyl, methylsulfonyl;

═O;

—OR¹⁹;

—N(R¹⁹)R²⁰;

—C(O)OR¹⁹;

—C(O)R¹⁹;

—C(O)N(R¹⁹)R²⁰;

—S(O)₂N(R¹⁹)R¹⁰; and/or

—N(R¹⁹)S(O)₂R²¹;

wherein R¹⁹ and R²⁰ independently represent, on each occasion when used above, hydrogen, phenyl, C₁₋₄ alkenyl (such as propenyl (e.g. propen-2-yl, i.e. allyl) or butenyl (e.g. but-3-enyl)), C₁₋₆ alkyl (such as methyl, ethyl, n-propyl, isopropyl, n-butyl or t-butyl) which alkyl group is optionally substituted by one or more fluoro atoms or a phenyl group;

R²¹ represents phenyl or C₁₋₆ alkyl (such as methyl, ethyl, n-propyl, isopropyl, n-butyl or t-butyl), which alkyl group is optionally substituted by one or more fluoro atoms.

When X represents an aryl or a heteroaryl group, then the substituents on such groups are preferably selected from carboxy, acetyl, methoxy, —NO₂, fluoro, methyl, chloro, hydroxymethyl, ethyl, isopropoxy, trifluoromethoxy and methylthio.

Preferred optional substituents on R¹ groups include phenoxy, trifluoromethyl, nitro, fluoro, chloro, cyano, carbamoyl, trifluoromethoxy, tetrazolyl (e.g. 2H-tetrazol-5-yl) and methyl.

When they represent an aryl or heteroaryl group, preferred optional substituents on R², R³, R⁴ or R⁵ groups include t-butyl, methylenedioxy, benzyloxy, nitro, methoxy, acetyl, chloro, fluoro, N-allyl-N-methanesulfonyl, cyano, trifluoromethyl, 2,2-dimethylpropionylamino, methanesulfonylamino, amino, but-3-enylamino, isopropoxy, methylthio, methylsulfonyl, ethenyl (i.e. vinyl), trifluoromethoxy, cyclohexyl, n-butyl, carboxy and hydroxymethyl.

Preferred optional substituents on R⁶ (when R⁶ does not represent hydrogen and is not linked to form a ring with R⁷) include an optionally substituted phenyl group. Optional substituents on such phenyl groups include halo (especially fluoro) and C₁₋₃ alkoxy (such as methoxy), which substituents are preferably in the 4-position of the phenyl ring.

Preferred optionally substituents on R⁷ (when R⁷ is not linked to form a ring with R⁶) include chloro, methoxy, amino, methyl, dimethylamino, phenyl, 4-methoxyphenyl, adamantyl, cyclohexyl, 3,3,5,5-tetramethylcyclohexyl, isopropoxy, trifluoromethyl, t-butyl, n-butyl, isopropyl, trifluoromethoxy, cyano, pyrrole (e.g. 2,5-dimethylpyrrole) and nitro.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:
(i) reaction of a compound of formula I¹, 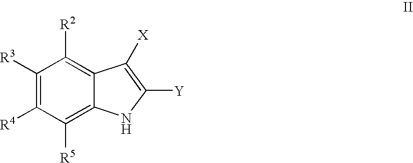
is wherein X, Y, R², R³, R⁴ and R⁵ are as hereinbefore defined, with a compound of formula III,
R¹ZL¹ III
wherein L¹ represents a suitable leaving group, such as halo (e.g. chloro, bromo or iodo), a carboxylate group, a sulfonylate group (e.g.—OS(O)₂CF₃, —OS(O)₂CH₃, —OS(O)₂PhMe or nonaflates), or an N-imidazolyl group and R¹ and Z are as hereinbefore defined, for example at around 0° C. to room temperature, or at above room temperature (e.g. up to 40-180° C.) in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, potassium hydroxide, Ar-ethyl-diisopropylamine, N-(methylpolystyrene)-4-(methylamino)pyridine, lithiumdiisopropylamide or mixtures thereof) and an appropriate solvent (e.g. tetrahydrofuran, pyridine, toluene, dichloromethane, chloroform, dimethylformamide, trifluoromethylbenzene, dimethylsulfoxide or triethylamine). Preferred base/solvent systems include sodium hydride/dimethylformamide, dimethylaminopyridine/pyridine, sodium hydroxide/dichloromethane (optionally in the presence of a phase transfer catalyst (e.g. tetrabutylammonium hydrogensulfate)), lithiumdiisopropylamide/tetrahydrofuran, potassium hydroxide/dimethylsulfoxide. Other systems that may be mentioned include sodium/ammonia.
(ii) reaction of a compound of formula IV, 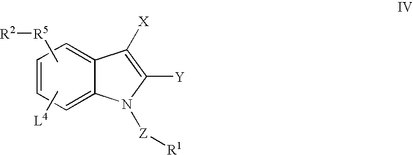
wherein L⁴ represents L² or L³, in which L² represents a leaving group such as halo (e.g. chloro, bromo or iodo), a sulfonylate group (e.g. —OS(O)₂CF₃, —OS(O)₂CH₃, —OS(O)₂PhMe or nonaflates) or —B(OH)₂, L³ represents a leaving group such as —B(OH)₂, -4,4,5,5-tetramethyl-1,2-dioxaborolan-2-yl, —SnBu₃, or a similar group known to the skilled person, L⁴ is attached to one or more of the carbon atoms of the benzenoid ring of the indole, and the remaining positions of the benzenoid ring are substituted with 1 to 3 (depending on the number of L⁴ substituents) substituents R² to R⁵ as appropriate, and Z, X, Y, R¹, R², R³, R⁴ and R⁵ are as hereinbefore defined, with a compound of formula V,
R²²L⁵ V
wherein R²² represents R², R³, R⁴ or R⁵ (as appropriate), and L⁵ represents L² (when L⁴ is L³) or L³ (when L⁴ is L²) as hereinbefore defined. The skilled person will appreciate that L² and L³ must be mutually compatible. This reaction may be performed, for example in the presence of a suitable catalyst system, e.g. a metal (or a salt or complex thereof) such as Cut, Pd/C, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-binaphthyl, 1,1′-bis(diphenylphosphinoferrocene), 1-bis(diphenylphosphino)propane or xantphos, together with a suitable base, such as Na₂CO₃, K₃PO₄, Cs₂CO₃, NaOH, K₂CO₃, CsF, Et₃N, (i-Pr)₂NEt or t-BuOK (or mixtures thereof) in a suitable solvent, such as dioxane, toluene, ethanol, dimethylformamide, ethylene glycol dimethyl ether, water, dimethylsulfoxide, acetonitrile, dimethylacetamide, N-methylpyrrolidinone or mixtures thereof. The reaction may also be carried out for example at room temperature or above (e.g. at a high temperature such as the reflux temperature of the solvent system) or using microwave irradiation;
(iii) for compounds of formula I, wherein X represents an optionally substituted aryl or heteroaryl group, reaction of a compound of formula VI, 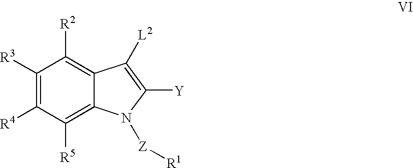
wherein Z, Y, L², R¹, R², R³, R⁴ and R⁵ are as hereinbefore defined, with a compound of formula VII,
X^{a}L³ VII
wherein X^{a} represents an aryl or heteroaryl group, optionally substituted as hereinbefore defined, and L³ is as hereinbefore defined, for example under reaction conditions such as those hereinbefore described hereinbefore in respect of process step (ii);
(iv) for compounds of formula I wherein X represents —N(R⁶)-E-R⁷, reaction of a compound of formula VI as hereinbefore defined, with a compound of formula VIII,
HN(R⁶)-E-R⁷ VIII
wherein E, R⁶ and R⁷ are as hereinbefore defined for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as Ph₃P, 2,2′-bis(diphenylphosphino)-1,1′-binaphthyl or xantphos, in the presence of an appropriate base such as Et₃N, pyridine, N,N′-dimethylethylenediamine, Na₂CO₃, K₃PO₄, Cs₂CO₃ or t-BuOK (or mixtures thereof), in a suitable solvent (e.g. dichloromethane, dioxane, toluene, ethanol, dimethylformamide, ethylene glycol dimethyl ether, water, dimethylsulfoxide, acetonitrile, dimethylacetamide, N-methylpyrrolidinone or mixtures thereof) or in the absence of an additional solvent when the reagent may itself act as a solvent (e.g. when R¹ represents phenyl and L¹ represents bromo, i.e. bromobenzene). This reaction may be carried out at room temperature or above (e.g. at a high temperature, such as the reflux temperature of the solvent system that is employed) or using microwave irradiation;
(v) for compounds of formula I wherein X represents —N(R⁶)-E-R⁷, reaction of a compound of formula IX, 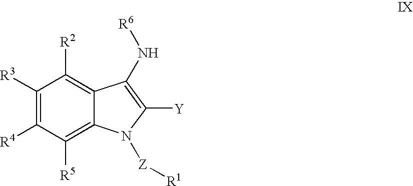
wherein Z, Y, R¹, R², R³, R⁴, R⁵ and R⁶ are as hereinbefore defined, with a compound of formula X,
R⁷-E-L¹ X
wherein E, R⁷ and L¹ are as hereinbefore defined, for example under reaction conditions such as those hereinbefore described in respect of process step (i); or
(vi) for compounds of formula I wherein E represents a single bond and R⁷ is a C₁₋₆ alkyl group, C₃₋₆ alkenyl or a C₃₋₆ alkynyl group, reduction of a compound of formula I, wherein X represents —C(O)— and R⁷ represents H, a C₁₋₅ alkyl group, a C₂₋₅ alkenyl or a C₂₋₅ alkynyl group, in the presence of a suitable reducing agent. A suitable reducing agent may be an appropriate reagent that reduces the amide group to the amine group in the presence of other functional groups (for example an ester or a carboxylic acid). Suitable reducing agents include borane and other reagents known to the skilled person, under reaction conditions known to the skilled person.

Compounds of formula II may be prepared by:
(a) reaction of a compound of formula XI, 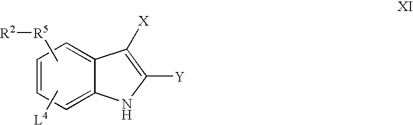
wherein X, Y, L⁴, R², R³, R⁴ and R⁵ are as hereinbefore defined with a compound of formula V for example under conditions such as those described hereinbefore in respect of process step (ii);
(b) for compounds of formula II wherein X represents an optionally substituted aryl or heteroaryl group, reaction of a compound of formula XII, 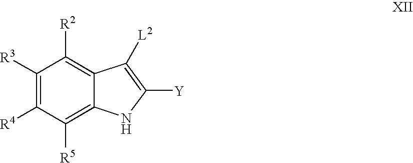
wherein Y, L², R², R³, R⁴ and R⁵ are as hereinbefore defined, with a compound of formula VII as hereinbefore defined, for example under conditions such as those described hereinbefore in respect of process step (iii);
(c) for compounds of formula II, wherein X represents —N(R⁶)-E-R⁷, reaction of a compound of formula XII as hereinbefore defined, with a compound of formula VIII for example under conditions such as those described hereinbefore in respect of process step (iv);
(d) for compounds of formula II, wherein X represents —N(R⁶)-E-R⁷, reaction of a compound of formula XIII, 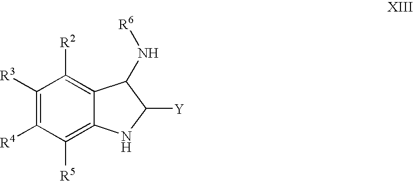
wherein Y, R², R³, R⁴, R⁵ and R⁶ are as hereinbefore defined with a compound of formula X for example under conditions such as those described hereinbefore in respect of process step (v);

Compounds of formula IV may be prepared by reaction of a compound of formula XI as hereinbefore defined, with a compound of formula III as hereinbefore defined for example under conditions such as those described hereinbefore in respect of process step (i).

Compounds of formula IV in which L⁴ represents L³ may be prepared by reaction of a compound of formula IV in which L⁴ represents L², with an appropriate reagent for the conversion of the L² group to the L³ group. This conversion may be performed by methods known to those skilled in the art, for example, compounds of formula IV, in which L³ is 4,4,5,5-tetramethyl-1,2-dioxaborolan-2-yl may be prepared by reaction of the reagent bis(pinacolato)diboron with a compound of formula IV in which L⁴ represents L², for example under reaction conditions such as those described hereinbefore in respect of process route (ii) above.

Compounds of formula VI may be prepared by:
(a) reaction of a compound of formula XII as hereinbefore defined with a compound of formula III as hereinbefore defined for example under conditions such as those described hereinbefore in respect of process step (i);
(b) for compounds of formula VI wherein L² represents a sulfonylate group, reaction of a compound of formula XV, 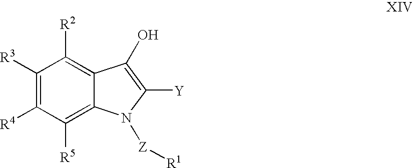
wherein Y, Z, R¹, R², R³, R⁴ and R⁵ are as hereinbefore defined, with an appropriate reagent for the conversion of the hydroxyl group to the sulfonylate group (e.g. tosyl chloride, mesyl chloride, triflic anhydride and the like) under conditions known to those skilled in the art.

Compounds of formula IX may be prepared for example by reaction of a compound of formula XIII as hereinbefore defined with a compound of formula III as hereinbefore defined for example under conditions such as those described hereinbefore in respect of process step (i).

Compounds of formula XII may be prepared by standard techniques. For example:
(a) Compounds of formula XII, wherein L² represents halo may be prepared by reaction of a compound of formula XV, 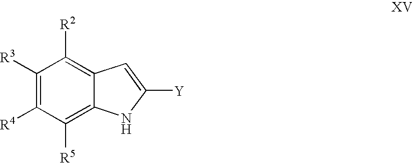
wherein R², R³, R⁴, R⁵ and Y are as hereinbefore defined, with a reagent, or mixture of reagents known to be a source of halide ions. For example, for bromide ions, N-bromosuccinimide may be employed, for iodide ions, iodine or a mixture of NaI and N-chlorosuccinimide may be employed, for chloride ions, N-chlorosuccinimide may be employed and for fluoride ions, 1-(chloromethyl)-4-fluoro-1,4-diazoniabicyclo[2.2.2]octane bis(tetrafluoroborate) may be employed. This reaction may be carried out in a suitable solvent (e.g. acetone or benzene) under conditions known to the skilled person.
(b) by reaction of a compound of formula XVI, 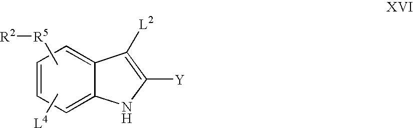
wherein Y, L², L⁴, R², R³, R⁴ and R⁵ are as hereinbefore defined with a compound of formula V as hereinbefore defined, for example under reaction conditions such as those described hereinbefore in respect of process route (ii) above; or
(c) for compounds of formula XII wherein L¹ represents a sulfonylate group, reaction of a compound of formula XVII, 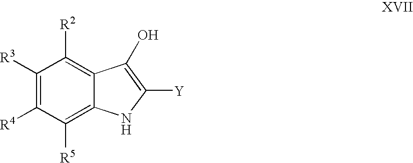
wherein Y, R², R³, R⁴ and R⁵ are as hereinbefore defined, with an appropriate reagent for the conversion of the hydroxyl group to a sulfonylate group as described hereinbefore.

Compounds of formula XM may be prepared by reaction of a compound of formula XII as hereinbefore defined with a compound of formula XVIII,
H₂NR⁶ XVIII
wherein R⁶ is as hereinbefore defined, for example under reaction conditions such as those described in respect of process step (iv).

Compounds of formula XII wherein R⁶ represents hydrogen may be prepared by an aromatic nitration reaction carried out on a compound of formula XV, as hereinbefore defined, followed by reduction of the nitro group of the resultant intermediate to an amino group. Both reactions may be performed under conditions known to the skilled person.

Compounds of formulae III, V, VII, VIII, X, XVIII are either commercially available, are known in the literature, or may be obtained either by analogy with the processes described herein, or by conventional synthetic procedures, in accordance with standard techniques, from available starting materials using appropriate reagents and reaction conditions.

Indoles of formulae II, IV, VI, IX, XI, XII, III, XIV, XV, XVI and XVII may also be prepared with reference to a standard heterocyclic chemistry textbook (e.g. “Heterocyclic Chemistry” by J. A. Joule, K. Mills and G. F. Smith, 3^{rd} edition, published by Chapman & Hall) and/or made according to the following general procedures.

For example compounds of formulae II, XI, XIII and XV may be prepared by reaction of a compound of formula XIX, 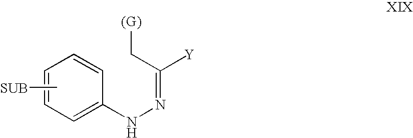
wherein SUB represents the substitution pattern that is present in the compound of formula II, XI, III or XV to be formed, (G) represents either X (as required for formation of compounds of formulae II and XI), a —N(R⁶)H group (as required for formation of compounds of formula XIII) or the (G) substituent is absent (as required for formation of compounds of formula XV) and Y is as hereinbefore defined, under Fischer indole synthesis conditions known to the person skilled in the art.

Compounds of formula XV may alternatively be prepared by reaction of a compound of formula XX, 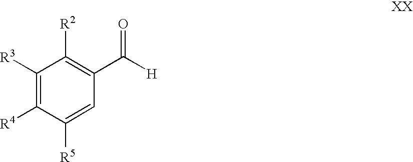
wherein R², R³, R⁴ and R⁵ are as hereinbefore defined with a compound of formula XXI,
N₃CH₂Y XXI
wherein Y is as hereinbefore defined, and preferably does not represent —COOH, under conditions known to the person skilled in the art (i.e. conditions to induce a condensation reaction, followed by a thermally induced cyclisation).

Compounds of formulae XV and XVII may be prepared by reaction of a compound of formula XXII, 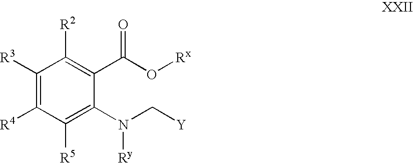
wherein R^{x} represents a C₁₋₆ alkyl group, R^{y} represents either -Z-R¹ as hereinbefore defined (as required for formation of compounds of formula XIV), hydrogen (as required for formation of compounds of formula XVII) or a nitrogen-protected derivative thereof, and Y, R², R³, R⁴ and R⁵ are as hereinbefore defined, under standard cyclisation conditions known to those skilled in the art.

Compounds of formulae IX and XIII, wherein R⁶ represents H, may be prepared by reaction of a compound of formula XXIII, 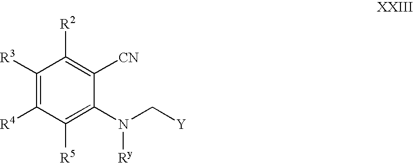
wherein Y, R², R³, R⁴, R⁵ and R^{y} are as hereinbefore defined, for example under intramolecular cyclisation conditions known to those skilled in the art.

Compounds of formula II and XI, wherein X represents aryl or heteroaryl, may alternatively be prepared by reaction of a compound of formula XXIV, 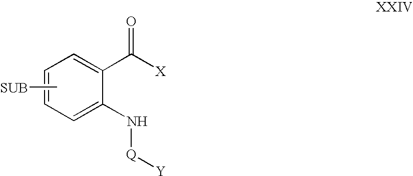
wherein Q represents either —(O)— or —CH₂—, X represents aryl or heteroaryl, and SUB and Y are as hereinbefore defined. When Q represents —C(O)—, the intramolecular cyclisation may be induced by a reducing agent such as TiCl₃/C₈K, TiCl₄/Zn or SmI₂ under conditions known to the skilled person, for example, at room temperature in the presence of a polar aprotic solvent (such as THF). When Q represents —CH₂—, the reaction may be performed in the presence of base under intramolecular condensation reaction conditions known to the skilled person.

Compounds of formula XIX may be prepared by:
(a) reaction of a compound of formula XXV, 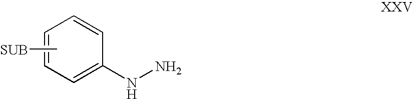
wherein SUB is as hereinbefore defined with a compound of formula XXVI, 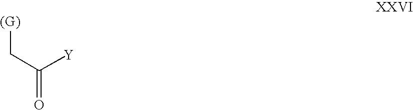
wherein (G) and Y are as hereinbefore defined under condensation conditions known to the skilled person; or
(b) reaction of a compound of formula XXVII, 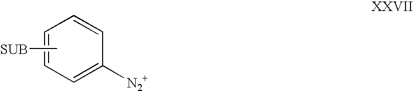
wherein SUB is as hereinbefore defined with a compound of formula XXVIII, 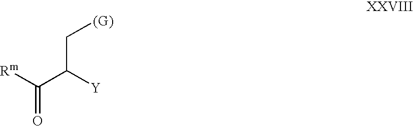
wherein R^{m} represents OH, O—C₁₋₆ alkyl, C₁₋₆ alkyl and (G) and Y are as hereinbefore defined, for example under Japp-Klingemann conditions known to the skilled person.

Compounds of formulae XX, XXI, XX XI, XI, XIV, XV, XVI, XVII and XVIII are either commercially available, are known in the literature, or may be obtained either by analogy with the processes described herein, or by conventional synthetic procedures, in accordance with standard techniques, from available starting materials using appropriate reagents and reaction conditions.

The substituents X, Y, Z, R¹, R², R³, R⁴ and R⁵ in final compounds of the invention or relevant intermediates may be modified one or more times, after or during the processes described above by way of methods that are well known to those skilled in the art. Examples of such methods include substitutions, reductions, oxidations, alkylations, hydrolyses, esterifications, and etherifications. The precursor groups can be changed to a different such group, or to the groups defined in formula I, at any time during the reaction sequence. For example, in cases where Y represent a carboxylic acid ester functional group, the skilled person will appreciate that at any stage during the synthesis (e.g. the final step), the relevant substituent may be hydrolysed so forming for example a carboxylic acid functional group. In cases where Y represents a carboxylic acid or carboxylic acid ester functional group, the relevant substituent may be reduced, under suitable conditions known to the skilled person (for example in the presence of other potentially reducible functional groups), at any stage during the synthesis (e.g. the final step), so forming for example a hydroxymethyl substituent.

Compounds of the invention may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chernistry”, edited by J W F McOrnie, Plenum Press (1973), and “Protective Groups in Organic Synthesis”, 3^{rd} edition, T. W. Greene & P. G. M. Wutz, Wiley-Interscience (1999).

Medical and Pharmaceutical Uses

Compounds of the invention are indicated as pharmaceuticals. According to a further aspect of the invention there is provided a compound of the invention for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which Y is —C(O)OR⁸ and R⁸ is other than hydrogen) may possess no or minimal pharmacological activity as such, but may be administered parenterally or orally, and thereafter be metabolised in the body to form compounds of the invention that possess pharmacological activity as such (including, but not limited to, corresponding compounds of formula I in which Y represents —COOH). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention are particularly useful because they may inhibit (for example selectively) the activity of prostaglandin E synthases (mPGESs) and particularly the activity of microsomal prostaglandin E synthase-1 (mPGES-1), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of inflammatory bowel disease, irritable bowel syndrome, migraine, headache, low back pain, fibromyalgia, myofascial disorders, viral infections (e.g. influenza, common cold, herpes zoster, and AIDS), bacterial infections, fungal infections, dysmenorrhea, burns, surgical or dental procedures, malignancies (e.g. breast cancer, colon cancer, and prostate cancer), atherosclerosis, gout, arthritis, osteoarthritis, juvenile arthritis, rheumatoid arthritis, rheumatic fever, ankylosing spondylitis, systemic lupus erythematosus, vasculitis, pancreatitis, nephritis, bursitis, conjunctivitis, iritis, scleritis, uveitis, wound healing, dermatitis, eczema, psoriasis, stroke, diabetes (e.g. diabetes mellitus), neurodegenerative disorders such as Alzheimer's disease and multiple sclerosis, autoimmune diseases, osteoporosis, asthma, chronic obstructive pulmonary disease, pulmonary fibrosis, allergic disorders, rhinitis, ulcers, coronary heart disease, sarcoidosis and any other disease with an inflammatory component.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a PGES (such as a mPGES, e.g. mPGES-1), and/or a method of treatment of a disease in which inhibition of the activity of a PGES, and particularly mPGES-1, is desired and/or required (e.g. inflammation), which method comprises administration of a therapeutically effective amount of a compound of the invention, as hereinbefore defined, to a patient suffering from, or susceptible to, such a condition,

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, intraperitoneally, topically (e.g. ocularly), intramuscularly, intraspinally, epidurally, transdermally, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:
(A) a compound of the invention, as hereinbefore defined; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of inflammation, and a pharmaceutically-acceptable adjuvant, diluent or carrier; and

(2) a kit of parts comprising components:

(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of prostaglandin E synthases (PGES) and particularly microsomal prostaglandin E synthase-1 (mPGES-1). The compounds of the invention may reduce the formation of the specific arachidonic acid metabolite PGE₂ without reducing the formation of other arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

Biological Test

In the assay mPGES-1 catalyses the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
180 μL per sample is analyzed with HPLC.

### EXAMPLES

The invention is illustrated by way of the following examples, in which the following abbreviations may be employed:

| | |
|---|---|
| BINAP | 2,2′-bis(diphenylphosphino)-1,1′-binaphthyl |
| dba | dibenzylideneacetone |
| DMAP | 4,4-dimethylaminopyridine |
| DME | ethylene glycol dimethyl ether |
| DMF | dimethylformamide |
| DMSO | dimethylsulfoxide |
| EtOAc | ethyl acetate |
| HPLC | High Pressure Liquid Chromatography |
| MeCN | acetonitrile |
| NBS | N-bromosuccinimide |
| NCS | N-chlorosuccinimide |
| NMR | nuclear magnetic resonance |
| TFA | trifluoroacetic acid |
| THF | tetrahydrofuran |
| TLC | thin layer chromatography |

Starting materials and chemical reagents specified in the syntheses described below are commercially available from, e.g. Sigma-Aldrich Fine Chemicals.

### Example 1

### 6-(4-tert-Butylphenyl)-1-(3-phenoxybenzyl)-3-phenylindole-2-carboxylic acid

### (a) 6-(4-tert-Butylphenyl)indole-2-carboxylic acid ethyl ester

A mixture of 6-bromoindole-2-carboxylic acid ethyl ester (400 mg, 1.5 mmol), 4-tert-butylphenylboronic acid (400 mg, 2.25 mmol), K₃PO₄ (950 mg, 1.5 mmol), Pd(OAc)₂ (18 mg, 0.075 mmol), 2,2′-bis(di-tertbutylphosphino)-1,1′-biphenyl (45 mg, 0.15 mmol), and toluene (9 mL) were stirred in an argon atmosphere for 30 min at room temperature, and at 100° C. for 40 min using microwave irradiation. The mixture was cooled to room temperature and poured into NaHCO₃ (aq., sat.). The mixture was extracted with EtOAc and the combined extracts were washed with water, brine and dried over Na₂SO₄. The organic phase was then concentrated and the product purified by chromatography to give the sub-title compound (392 mg, 81%).

### (b) 6-(4-tert-Butylphenyl)-3-iodoindole-2-carboxylic acid ethyl ester

The reaction was performed with the exclusion of light. A solution of NaI (300 mg, 2.0 mmol) in acetone (15 mL) was added dropwise to a stirred solution of NCS (270 mg, 2.0 mmol) in acetone (4 mL), followed after 15 min by the dropwise addition of 6-(4-tert-butylphenyl)indole-2-carboxylic acid ethyl ester (650 mg, 2.0 mmol; see step (a) above) in acetone (20 mL). After 30 min at room temperature the mixture was poured into an aqueous solution of Na₂S₂O₃ (aq., 10%) and extracted with EtOAc. The combined extracts were washed with water, brine and dried over Na₂SO₄. The organic phase was then concentrated and then purified by chromatography to give the sub-title compound (743 mg, 82%).

### (c) 6-(4-tert-Butylphenyl)-3-iodo-1-(3-phenoxybenzyl)-indole-2-carboxylic acid ethyl ester

A solution of 6-(4-tert-butylphenyl)-3-iodoindole-2-carboxylic acid ethyl ester (743 mg, 1.66 mmol; see step (b) above) in DMF (10 mL) was added carefully to a stirred suspension of NaH (41 mg, 1.69 mmol) in DMF (4 mL) at 0° C. The mixture was stirred at room temperature for 25 min. A solution of 3-phenoxybenzyl chloride (378 mg, 1.69 mmol) in DM (6 mL) was then added in portions and the mixture was stirred at room temperature for a further 24 h, then poured into water and extracted with t-BuOMe. The combined extracts were washed with water, brine and dried over Na₂SO₄. The organic phase was concentrated and the product purified by chromatography and then crystallisation from EtOH to give the sub-title compound (766 mg, 73%).

### (d) 6-(4-tert-Butylphenyl)-1-(3-phenoxybenzyl)-3-phenylindole-2-carboxylic acid ethyl ester

A mixture of 6-(4-tert-butylphenyl)-3-iodo-1-(3-phenoxybenzyl)-indole-2-carboxylic acid ethyl ester (200 mg, 0.32 mmol; see step (c) above), phenylboronic acid (59 mg, 0.48 mmol), K₃PO₄ (238 mg, 1.12 mmol), Pd(OAc)₂ (3.6 mg, 0.016 mmol) and toluene (3 mL) was stirred for 20 min at room temperature and then for 4 h at 80° C. The mixture was poured into NaHCO₃ (aq., sat.) and extracted with EtOAc. The combined extracts were washed with water, brine and dried over Na₂SO₄. The organic phase was then concentrated and the product purified by column chromatography to give the sub-title compound (163 mg, 88%).

### (e) 6-(4-tert-Butylphenyl)-1-(3-phenoxybenzyl)-3-phenylindole-2-carboxylic acid

A mixture of 6-(4-tert-butylphenyl)-1-(3-phenoxybenzyl)-3-phenylindole-2-carboxylic acid ethyl ester (163 mg, 0.281 mmol; see step (d)), aqueous NaOH (1 M, 10 mL) and MeCN (40 mL) was heated at reflux for 4 h. The mixture was then allowed to cool, acidified with HCl (1M) to pH 2 and extracted with EtOAc. The combined extracts were washed with water, brine and dried over Na₂SO₄. The combined extracts were concentrated and the product was purified by chromatography and recrystallisation firstly from EtOH and then from MeCN to give the title compound (95 mg, 61%).

¹HNMR (DMSO-d₆, 200 MHz): δ 7.61-7.37 (12H, m), 7.29-7.17 (3H, m), 7.09-7.00 (1H, m), 6.97-6.90 (2H, m), 6.85 (1H, d, J=2.0 Hz), 6.83-6.77 (2H, m), 5.86 (2H, s), 1.37 (9H, s).

### Example 2

### 6-(4-tert-Butylphenyl)-1-(3-phenoxybenzyl)-3-(2-thienyl)indole-2-carboxylic acid

### (a) 6-(4-tert-Butylphenyl)-1-(3-phenoxybenzyl)-3-(2-thienyl)indole-2-carboxylic acid ethyl ester

2-(Tributylstannyl)thiophene (72 mg, 0.20 mmol) was added to a stirred mixture of 6-(4-tert-butylphenyl)-3-iodo-1-(3-phenoxybenzyl)-indole-2carboxylic acid ethyl ester (150 mg, 0.24 mmol; see Example 1(d)), CuI (25 mg, 0.13 mmol), Pd(PPh₃)₂Cl₂ (18 mg, 0.026 mmol) and DMF (3 mL). After 10 min at room temperature and 1 h at 90° C. another portion of 2-(tributylstannyl)thiophene (72 mg, 0.20 mmol) was added and the heating was continued for 3 h. The mixture was filtered through Celite® and the solids were washed with EtOAc. Concentration and purification by chromatography gave the sub-title compound (125 mg, 90%).

### (b) 6-(4-tert-Butylphenyl)-1-(3-phenoxybenzyl)-3-(2-thienyl)indole-2-carboxylic acid ethyl ester

A mixture of 6-(4-tert-butylphenyl)-1-(3-phenoxybenzyl)-3-phenylindole-2-carboxylic acid ethyl ester (125 mg, 0.213 mmol; see step (a)), aqueous KOH (2M, 2 mL) and MeCN (6 mL) was heated for 30 min at 130° C. using microwave irradiation. The mixture was acidified with HCl (1M) to pH 2 and extracted with EtOAc. The combined extracts were washed with water, brine and dried over Na₂SO₄. Concentration and purification by chromatography gave the title compound (91 mg, 77%). ¹H NMR (DMSO-d₆, 200 MHz): δ 7.75 (1H, d, J=8.4 Hz), 7.56-7.45 (6H, m), 7.44 (1H, dd, J=4.0, 1.4 Hz), 7.30-7.15 (5H, m), 7.09-7.03 (1H, m), 6.98-6.90 (2H, m), 6.86-6.79 (3H, m), 5.86 (2H, s), 1.37 (9H, s).

### Example 3

### 5-(3,4-Methylenedioxyphenyl)-3-phenyl-1-(3-phenylpropyl)indole-2-carboxylic acid

### (a) N-(2-Benzoyl-4-chlorophenyl)oxalamic acid ethyl ester

A mixture of 2-amino-5-chlorobenzophenone (11.6 g, 50 mmol), ethyl oxalylchloride (6.8 g, 50 mmol) and toluene (70 mL) was heated at reflux for 1.5 h. On cooling a yellow precipitate formed. EtOAc (250 mL) was added and the solution was washed with NaHCO₃ (aq., 5%), H₃PO₄ (aq., 5%), brine and dried over Na₂SO₄. Concentration gave the sub-title compound (15.5 g, 94%).

### (b) 5-Chloro-3-phenylindole-2-carboxylic acid ethyl ester

TiCl₄ in THF (0.25 M, 19.5 mL, 54.9 mmol) was added slowly to a stirred mixture of N-(2-benzoyl-4-chlorophenyl)oxalamic acid ethyl ester (8.88 g, 26.8 mmol; see step (a)), Zn (7.19 g, 110 mmol) and THF (60 mL) at room temperature. After 2 h, silica gel was added and then after a further 30 min the mixture was filtered through a pad of silica gel which was washed with EtOAc. The combined filtrates were washed with NaHCO₃ (aq., 5%), water, brine and dried over Na₂SO₄. Concentration and crystallisation of the residue from CH₂Cl₂/petroleum ether gave the title compound (3.13 g, 39%).

### (c) 5-Chloro-3-phenyl-1-(3-phenylpropyl)indole-2-carboxylic acid ethyl ester

NaH (60% dispersion in mineral oil, 0.25 g, 6.2 mmol) was washed with hexane (2×1 mL) and Et₂O (1 mL) and suspended in DMF (1 mL). A solution of 5-chloro-3-phenylindole-2-carboxylic acid ethyl ester (1.55 g, 5.17 mmol; see step (b)) in DMF (10 mL) was added carefully at 0° C. and the mixture was stirred for 20 min. A solution of (3-bromopropyl)-benzene (1.54 g, 7.75 mmol) in DMF (3 mL) was added carefully at 0° C. The cooling bath was removed and the mixture was stirred at room temperature for 16 h, poured into water, and extracted with EtOAc. The extract was washed with water, brine and dried over Na₂SO₄. Concentration and chromatography gave the title compound (1.66 g, 77%).

### (d) 5-(3,4-Methylenedioxyphenyl)-3-phenyl-1-(3-phenylpropyl)indole-2-carboxylic acid

The title compound was prepared in accordance with the procedure in Example 1(a) from 5-chloro-3-phenyl-1-(3-phenylpropyl)indole-2-carboxylic acid ethyl ester (see step (c)) and 3,4-methylenedioxyphenylboronic acid, followed by hydrolysis in accordance with the procedure described in Example 2(b).

¹HNMR (CDCl₃, 200 MHz): δ 7.61-7.38 (8H, m), 7.36-7.15 (5H, m), 7.04 (1H, s), 7.02 (1H, dd, J=8.5, 1.8 Hz), 6.85 (1H, d, J=8.5 Hz), 5.98 (2H, s), 4.67-4.55 (2H, m), 2.75 (2H, t, J=7.6 Hz), 2.31-2.12 (2H, m).

### Example 4

### 3-Phenyl-1-(3-phenylpropyl)-5-(3-pyridyl)indole-2-carboxylic acid

### (a) 3-Phenyl-1-(3-phenylpropyl)-5-(4,4,5,5-tetramethyl-1,2-dioxaborolan-2-yl)indole-2-carboxylic acid ethyl ester

A 0.01 M stock solution of a Pd/(C₆H₁₁)₃P was prepared from Pd₂(dba)₃, (0.457 g, 0.5 mmol), tricyclohexylphosphine (0.841 g, 3 mmol) and dioxane (100 mL). An aliquot of this stock solution (12.5 mL, 0.125 mmol Pd), 5-chloro-3-phenyl-1-(3-phenylpropyl)indole-2-carboxylic acid ethyl ester (1.05 g, 2.5 mmol; see Example 3(c)), bis(pinacolato)diboron (0.762 g, 3.0 mmol), KOAc (0.44 g, 4.5 mmol), and dioxane (25 mL) were heated at 80° C. for 16 h. Another aliquot of the Pd/(C₆H₁₁)₃P reagent (2.5 mL, 0.025 mmol Pd) was added and the mixture was heated at 100° C. for 24 h. The mixture was filtered through Celite®, and the filtrate was concentrated and purified by chromatography to give the sub-title compound (0.47 g, 37%) together with 0.55 g recovered starting material.

### (b) 3-Phenyl-1-(3-phenylpropyl)-5-(3-pyridyl)indole-2-carboxylic acid ethyl ester

3-Phenyl-1-(3-phenylpropyl)-5-(4,4,5,5-tetramethyl-1,2-dioxaborolan-2-yl)indole-2-carboxylic acid ethyl ester (0.40 g, 91 mmol; see step (a)), 3-iodopyridine (0.28 g, 1.37 mmol), aqueous Na₂CO₃ (2M, 0.46 mL, 0.91 mmol), Pd(PPh₃)₄ (53 mg, 46 μmol), toluene (7.3 mL), and EtOH (1.8 mL) were heated at 80° C. for 16 h. More 3-iodopyridine (0.19 g, 0.91 mmol), aqueous Na₂CO₃ (2M, 1.4 mL, 2.73 mmol), and Pd(PPh₃)₄ (23 mg, 20 μmol) were added and the mixture was heated for a further 8 h. EtOAc (30 mL) and brine (30 mL) were added. The layers were separated and the aqueous phase was washed with EtOAc. The combined organic phases were dried with brine and Na₂SO₄. Concentration and chromatography gave the sub-title compound (0.32 g, 76%).

### (c) 3-Phenyl-1-(3-phenylpropyl)-5-(3-pyridyl)indole-2-carboxylic acid

The title compound was prepared from 3-phenyl-1-(3-phenylpropyl)-5-(3-pyridyl)indole-2-carboxylic acid ethyl ester (see step (b)) in accordance with the procedure in Example 2(b).

¹HNMR (CDCl₃, 200 MHz): δ 8.78 (1H, s), 8.52 (1H, d, J=4.2 Hz), 7.92 (1H, d, J=8.0 Hz)), 7.65-7.71 (1H, m), 7.59-7.13 (13H, m), 4.71-4.58 (2H, m), 2.75 (2H, t, J=7.6 Hz), 2.32-2.17 (2H, m).

### Example 5

### 6-(4-Benzyloxyphenyl)-3-(3-carboxyphenyl)-1-(3-nitrobenzyl)indole-2-carboxylic acid

The title compound was prepared in accordance with Example 1 from 4-benzyloxyphenylboronic acid, 3-nitrobenzylbromide and 3-carboxyphenylboronic acid.

¹HNMR (DMSO-d₆, 200 MHz): δ 13.02 (2H, s), 8.13-7.94 (5H, m), 7.75 (1H, ddd, J=7.6, 1.5, 1.5 Hz), 7.63 (1H, dd, J=7.6, 2.0 Hz), 7.59-7.27 (12H, m), 7.04-6.97 (1H, m), 6.12 (2H, s), 5.18 (2H, s).

### Example 6

### 3-(3-Carboxyphenyl)-4-phenyl-1-[3-(trifluoromethyl)benzyl]indole-2-carboxylic acid

The title compound was prepared in accordance with Example 1 from 4-bromoindole-2-carboxylic acid ethyl ester, phenylboronic acid, 3-(trifluoromethyl)benzylbromide and 3-carboxyphenylboronic acid.

¹HNMR (CDCl₃, 200 MHz): δ 7.79 (1H, dd, J=1.5, 1.5 Hz), 7.70 (1H, ddd, J=7.7, 1.5, 1.5 Hz), 7.57-7.50 (2H, m), 7.47-7.34 (3H, m), 7.25-7.20 (1H, m), 7.06 (1H, dd, J=6.2, 1.8 Hz), 7.06-6.85 (7H, m), 5.93 (2H, s).

### Example 7

### 6-(4-Benzyloxyphenyl)-1-(3-nitrobenzyl)-3-(2-oxopyrrolidin-1-yl)indole-2-carboxylic acid

### (a) 6-(4-Benzyloxyphenyl)-1-(3-nitrobenzyl)-3-(2-oxopyrrolidin-1-yl)indole-2-carboxylic acid ethyl ester

A stock suspension of a CuI/MeNHCH₂CH₂NHMe complex was prepared by heating CuI (95.2 mg, 0.5 mmol), MeNHCH₂CH₂NHMe (213 μL, 2.0 mmol), and dioxane (5 mL) at 100° C. for 5 min using microwave irradiation. 1.5 mL of this solution was added to 6-(4-benzyloxyphenyl)-3-iodo-1-(3-nitrobenzyl)indole-2-carboxylic acid ethyl ester (630 mg, 1.0 mmol, prepared in accordance with Example 1 from 6-bromoindole-2-carboxylic acid ethyl ester, 4-benzyloxyphenylboronic acid, and 3-nitrobenzylbromide), K₃PO₄ (530 mg, 2.5 mmol), and dioxane (5 mL). Pyrrolidinone (390 mg, 5.0 mmol) was added and the mixture was stirred at 95° C. for 24 h, cooled to room temperature, poured into aqueous HCl (0.1M) and extracted with EtOAc. The combined extracts were dried with brine and Na₂SO₄. Concentration and chromatography gave the sub-title compound (538 mg, 91%).

### (b) 6-(4-Benzyloxyphenyl)-1-(3-nitrobenzyl)-3-(2-oxopyrrolidin-1-yl)indole-2-carboxylic acid

6-(4-Benzyloxyphenyl)-1-(3-nitrobenzyl)-3-(2-oxopyrrolidin-1-yl)indole-2-carboxylic acid ethyl ester (see step (a)) was hydrolyzed in accordance with Example 2(b), using dioxane as the solvent, to give the title compound.

¹HNMR (DMSO, 200 MHz): δ 13.37 (1H, s), 8.10-8.06 (2H, m), 7.96 (1H, s), 7.66-7.26 (12H, m), 7.00 (1H, d, J=7.8 Hz), 6.06 (2H, s), 5.17 (2H, s), 3.81-3.74 (2H, m), 2.46-2.38 (2H, m), 2.24-2.06 (2H, m).

### Example 8

### 3-(2-Oxopyrrolidin-1-yl)-1-(3-phenoxybenzyl)-5-phenylindole-2-carboxylic acid

The title compound was prepared in accordance with Example 7 from 5-bromoindole-2-carboxylic acid ethyl ester, phenylboronic acid, 3-phenoxybenzylchloride and pyrrolidinone.

¹HNMR (CDCl₃, 200 MHz): δ 7.69-7.65 (1H, m), 7.63-7.52 (3H, m), 7.50-7.15 (7H, m), 7.13-7.01 (1H, m), 7.00-6.91 (2H, m), 6.88-6.79 (3H, m), 5.74 (2H, s), 3.98 (2H, t, J=7.0 Hz), 2.72 (2H, t, J=8.0 Hz), 2.41-2.34 (2H, m).

### Example 9

### 1-(3,5-Difluorobenzyl)-4-methoxy-3-(2-oxopyrrolidin-1-yl)-7-phenylindole-2-carboxylic acid

### (a) 2-Azido-3-(4-methoxybiphenyl-3-yl)acrylic acid ethyl ester

A solution of 4-methoxybiphenyl-3-carboxaldehyde (1.8 g, 8.48 mmol) and azidoacetic acid ethyl ester (5.62 g, 44 mmol) in EtOH (15 mL) was added dropwise to a solution of NaOEt (3.13 g, 46 mmol) in EtOH (35 mL) at −25° C. The mixture was stirred at that temperature for 10 min, kept in the freezer (−18° C.) for 24 h and then poured whilst stirring vigorously to a cooled (0° C.) solution of NH₄Cl (aq., sat.). The mixture was extracted with EtOAc and the combined extracts were washed with brine and dried over Na₂SO₄. Concentration and crystallisation from EtOH gave the title compound (1.80 g, 66%).

### (b) 4-Methoxy-7-phenylindole-2-carboxylic acid ethyl ester

A solution of 2-azido-3-(4-methoxybiphenyl-3-yl)acrylic acid ethyl ester (1.75 g, 5.40 mmol; see step (a)) in o-xylene (25 mL) was added dropwise to boiling o-xylene (25 mL). The heating was continued for 5 min, then the solution was allowed to cool to room temperature and kept in the freezer (−18° C.) for 16 h. The precipitate was isolated by filtration, washed with petroleum ether and dried in vacuo to afford the sub-title compound (1.20 g, 74%).

### (c) 1-(3,5-Difluorobenzyl)-4-methoxy-3-(2-oxopyrrolidin-1-yl)-7-phenylindole-2-carboxylic acid

The title compound was prepared from 4-methoxy-7-phenylindole-2-carboxylic acid ethyl ester (see step (b)) in accordance with Example 7.

¹HNMR (DMSO-d₆, 200 MHz): δ 7.34-7.21 (3H, m), 7.10-7.06 (2H, m), 7.01-6.91 (1H, m), 6.94 (1H, d, J=7.8 Hz), 6.67 (1H, d, J=8.1 Hz), 5.92-5.89 (2H, m), 5.51-5.28 (2H, m), 3.89 (3H, s), 3.77-3.67 (2H, m), 2.37-2.17 (2H, m), 2.14-2.10 (2H, m).

### Example 10

### 6-(3,4-Methylenedioxyphenyl)-1-[3,5-bis(trifluoromethyl)benzyl]-3-(4-chlorobenzoylamino)indole-2-carboxylic acid

The title compound was prepared according in accordance with Example 7 from 6-bromoindole-2-carboxylic acid ethyl ester, 3,4-methylenedioxyphenylboronic acid, 3,5-bis(trifluoromethyl)benzylchloride and 4-chlorobenzamide.

¹HNMR (DMSO-d₆, 200 MHz): δ 10.35 (1H, s), 8.09-8.03 (2H, m), 8.01-7.99 (1H, m), 7.95-7.94 (1H, m), 7.81 (2H, s), 7.73 (1H, d, J=8.6 Hz), 7.66-7.59 (2H, m), 7.44 (1H, dd, J=8.6, 1.2 Hz), 7.34 (1H, d, J=1.8 Hz), 7.22 (1H, dd, J=8.2, 1.8 Hz), 6.99 (1H, d, J=8.2 Hz), 6.08 (2H, s), 6.05 (2H, s).

### Example 11

### 3-(3,5-Dimethoxybenzoylamino)-5-(4-nitrophenyl)-1-(3-phenoxybenzyl)indole-2-carboxylic acid

The title compound was prepared from 5-(4-nitrophenyl)indole-2-carboxylic acid ethyl ester prepared from 5-(4,4,5,5-tetramethyl-1,2-dioxaborolan-2-yl)indole-2-carboxylic acid ethyl ester (prepared from 5-bromoindole-2-carboxylic acid ethyl ester) and 4-nitrobromobenzene), 3-phenoxybenzylchloride and 3,5-dimethoxybenzamide in accordance with Example 7.

¹HNMR (DMSO-d₆, 200 MHz): δ 10.37 (1H, s), 8.32-8.24 (2H, m), 8.15 (1H, s), 8.01-7.91 (2H, m), 7.76 (2H, s), 7.41-7.16 (5H, m), 7.15-7.05 (1H, m), 6.99-6.90 (2H, m), 6.84-6.69 (4H, m), 5.90 (2H, s), 3.81 (6H, s).

### Example 12

### 3-(3-Amino-4-methylbenzoylamino)-5-(4-tert-butylphenyl)-1-(3-chlorobenzyl)indole-2-carboxylic acid

The title compound was prepared in accordance with Example 7 from 5-bromoindole-2-carboxylic acid ethyl ester, 4-tert-butylphenylboronic acid, 3-chlorobenzylchloride and 3-amino-4-methylbenzamide.

¹H NMR (DMSO-d₆, 200 MHz): δ 10.09 (1H, s), 8.00 (1H, s), 7.69-7.40 (6H, m), 7.36-7.31 (3H, m), 7.19-6.94 (3H, m), 5.86 (2H, s), 4.3 (1H, br s), 3.3 (1H, br s), 2.11 (3H, s), 1.29 (9H, s).

### Example 13

### 5-(4-tert-Butylphenyl)-1-(3-chlorobenzyl)-3-[(pyridine-3-carbonyl)amino]-indole-2-carboxylic acid

The title compound was prepared in accordance with Example 7 from 5-bromoindole-2-carboxylic acid ethyl ester, 4-tert-butylphenylboronic acid, 3-chlorobenzylchloride and nicotinamide.

¹HNMR (DMSO-d₆, 200 MHz): δ 10.50 (1H, s), 9.20 (1H, s), 8.80-8.71 (1H, m), 8.42-8.31 (1H, m), 7.91 (1H, s), 7.32-7.52 (5H, m), 7.48-7.40 (2H, m), 7.34-7.27 (2H, m), 7.14 (1H, s), 7.06-6.97 (1H, m), 5.89 (2H, s), 1.29 (9H, s).

### Example 14

### 3-[4-(Dimethylamino)butyrylamino]-6-(3,4-methylenedioxphenyl)-1-(3-phenoxybenzyl)indole-2-carboxylic acid

The title compound was prepared in accordance with Example 7 from 6-bromoindole-2-carboxylic acid ethyl ester, 3,4-methylenedioxyphenylboronic acid, 3-phenoxybenzylchloride and 4-(dimethylamino)butyrylamide.

¹HNMR (DMSO-d₆, 200 MHz): δ 12.3-11.2 (1H, br s), 8.22 (1H, d, J=8.6 Hz), 7.54 (1H, s), 7.37-7.05 (7H, m), 7.00-6.84 (5H, m), 6.71 (1H, dd, J=8.2, 2.3 Hz), 6.14 (2H, s), 6.07 (2H, s), 2.81 (2H, t, J=7.5 Hz), 2.54 (6H, s), 2.46 (2H, t, J=7.3 Hz), 1.94 (2H, dt, J=7.5, 7.3 Hz).

### Example 15

### 1-(3-Cyanobenzyl)-6-(3,4-methylenedioxyphenyl)-3-(3-phenylacryloylamino)indole-2-carboxylic acid

The title compound was prepared in accordance with Example 7 from 6-bromoindole-2-carboxylic acid ethyl ester, 3,4-methylenedioxyphenylboronic acid, 3-cyanobenzylchloride and cinnamamide.

¹H NMR (DMSO-d₆, 200 MHz): δ 11.95 (1H, br s), 8.28 (1H, d, J=8.6 Hz), 7.70-7.59 (6H, m), 7.54-7.37 (6H, m), 7.28-7.23 (2H, m), 7.15 (1H, dd, J=8.2, 1.8 Hz), 6.96 (1H, d, J=8.2 Hz), 6.88 (1H, d, J=15.8 Hz), 6.16 (2H, s), 6.03 (2H, s).

### Example 16

### 1-(3-Carbamoylbenzyl)-6-(3,4-methylenedioxyphenyl)-3-(3-phenylacryloylamino)indole-2-carboxylic acid

The title compound was isolated in the last synthetic step (the hydrolysis) in the preparation of the title compound of Example 15.

¹HNMR (DMSO-d₆, 200 MHz): δ 11.58 (1H, br s), 8.17 (1H, d, J=8.6 Hz), 7.89 (1H, s), 7.74-7.55 (6H, m), 7.48-7.37 (3H, m), 7.34-7.23 (4H, m), 7.16-7.08 (2H, m), 6.95 (1H, d, J=8.2 Hz), 6.91 (1H, d, J=15.6 Hz), 6.13 (2H, s), 6.03 (2H, s).

### Example 17

### 3-Acetylamino-5-(3,4-methylenedioxyphenyl)-1-(5-phenoxypentyl)indole-2-carboxylic acid

The title compound was prepared in accordance with Example 7 from 5-bromoindole-2-carboxylic acid ethyl ester, 3,4-methylenedioxyphenylboronic acid, (5-bromopentyloxy)benzene, and acetamide.

¹H NMR (DMSO-d₆, 200 MHz): δ 9.60 (1H, s), 7.71 (1H, s), 7.65-7.49 (2H, m), 7.30-7.15 (3H, m), 7.09 (1H, dd, J=8.1, 1.4 Hz), 6.77 (1H, d, J=8.1 Hz), 6.93-6.82 (3H, m), 6.04 (2H, s), 4.60-4.46 (2H, m), 3.90 (2H, t, J=6.3 Hz), 2.09 (3H, s), 1.81-1.61 (4H, m), 1.48-1.32 (2H, m).

### Example 18

### 5-(3,4-Methylenedioxyphenyl)-3-(2-oxopiperidin-1-yl)-1-(5-phenoxypentyl)indole-2-carboxylic acid

The title compound was prepared in accordance with Example 7 from 5-bromoindole-2-carboxylic acid ethyl ester, 3,4-methylenedioxyphenylboronic acid, (5-bromopentyloxy)benzene, and 2-piperidone.

¹H NMR (DMSO-d₆, 200 MHz): δ 7.66-7.49 (3H, m), 7.30-7.18 (3H, m), 7.13 (1H, dd, J=8.1, 1.8 Hz), 6.97 (1H, d, J-8.1 Hz), 6.92-6.83 (3H, m), 6.04 (2H, s), 4.65-4.50 (2H, m), 3.91 (2H, t, J=6.4 Hz), 3.64-3.55 (2H, m), 2.44-2.28 (2H, m), 1.98-1.64 (8H, m), 1.52-1.33 (2H, m).

### Example 19

### 3-(3-Acetylphenyl)-5-(4-methoxyphenyl)-1-[3-(trifluoromethyl)benzyl]-indole-2-carboxylic acid

### (a) 5-(4-Methoxyphenyl)indole-2-carboxylic acid ethyl ester

A mixture of 5-bromoindole-2-carboxylic acid ethyl ester (268 mg, 1.0 mmol), 4-methoxyphenylboronic acid (243 mg, 1.6 mmol), Pd(OAc)₂ (11.2 mg, 50 nmol), 2-(di-t-butylphosphino)biphenyl (60 mg, 200 mmol), K₃PO₄ (425 mg, 2 mmol) and toluene (2 mL) was heated at 170° C. for 10 min using microwave irradiation. The mixture was extracted with EOAc, and the combined extracts washed with water, dried with Na₂SO₄, and concentrated. The residue was crystallised from EtOH to yield the sub-title compound (50%).

### (b) 3-Iodo-5-(4-methoxyphenyl)indole-2-carboxylic acid ethyl ester

NaI (566 mg, 3.8 mmol) in acetone (18 mL) was added dropwise to a solution of NCS (458.5 mg, 3.4 mmol) in acetone (6 mL) at room temperature. After 15 min, 5-(4-methoxyphenyl)indole-2-carboxylic acid ethyl ester (1.01 g, 3.4 mmol; see step (a)) in acetone (48 mL) was added. After 60 min Na₂S₂O₃ (aq., 10%) was added and the mixture was extracted with EtOAc, and the combined extracts washed with water, dried with Na₂SO₄, and concentrated. The residue was crystallised from EtOAc/heptane to yield the sub-title compound (958 mg, 66%)

### (c) 3-Iodo-5-(4-methoxyphenyl)-1-[3-(trifluoromethyl)benzyl]indole-2-carboxylic acid ethyl ester

A mixture of 3-iodo-5-(4-methoxyphenyl)indole-2-carboxylic acid ethyl ester (200 mg, 475 nmol; see step (b)), 1-(bromomethyl)-3-(trifluoromethyl)benzene (182 μL, 1.2 mmol), NaH (48 mg, 2 mmol) and DMF (2.5 mL) was heated at 170° C. for 2 min using microwave irradiation. The mixture was extracted with EOAc. The combined extracts were washed with water, dried with Na₂SO₄ and concentrated. The residue was crystallised from EtOH to yield the sub-title compound (several batches were combined and employed in subsequent steps).

### (d) 3-(3-Acetylphenyl)-5-(4-methoxyphenyl)-1-[3-(trifluoromethyl)benzyl]-indole-2-carboxylic acid ethyl ester

A mixture of 3-iodo-5-(4-methoxyphenyl)-1-[3-(trifluoromethyl)benzyl]-indole-2-carboxylic acid ethyl ester (100 mg, 173 nmol; see step (c)), 3-acetylphenylboronic acid (42.5 mg, 259 nmol), Na₂CO₃ (27 mg, 259 nmol), Pd(PPh₃)₂Cl₂ (6.1 mg, 9 mmol) and DME/H₂O/EtOH 7:3:2 (1 mL) was heated at 160° C. for 10 minutes using microwave irradiation. The reaction mixture was filtered through Celite® and the filter cake was washed with Et₂O. The combined filtrates were poured into NaHCO₃ (aq., sat.) and extracted with Et₂O. The combined extracts were dried over Na₂SO₄ and concentrated. Purification by chromatography yielded the sub-title compound (74 mg, 71%).

### (e) 3-(3-Acetylphenyl)-5-(4-methoxyphenyl)-1-[3-(trifluoromethyl)benzyl]-indole-2-carboxylic acid

A mixture of 3-(3-acetylphenyl)-5-(4-methoxyphenyl)-1-[3-(trifluoromethyl)benzyl]indole-2-carboxylic acid ethyl ester (59 mg, 103 nmol; see step (d)), NaOH (2 M, 500 μL) and MeCN (2 mL) was heated at 120° C. for 10 min using microwave irradiation. The mixture was acidified with HCl (2M) and extracted with EOAc. The combined extracts were dried and concentrated. Purification by chromatography yielded the title compound.

¹HNMR (200 MHz, CDCl₃): δ 8.10 (m, 1H), 7.99 (m, 1H), 7.71 (m, 1H), 7.63-7.54 (m, 3H), 7.53-7.47 (m, 3H), 7.46 (m, 1H), 7.40 (m, 1H), 7.35 (d, J=8 Hz, 1H), 7.18 (d, J=8 Hz, 1H), 6.94 (m, 2H), 5.88 (s, 2H), 3.83 (s, 3H), 2.61 (s, 3H).

### Example 20

### 5-(4-Methoxyphenyl)-3-phenyl-1-[3-(trifluoromethyl)benzyl]indole-2-carboxylic acid

### (a) 5-(4-Methoxyphenyl)-3-phenyl-1-[3-(trifluoromethyl)benzyl]indole-2-carboxylic acid ethyl ester

A mixture of 3-iodo-5-(4-methoxyphenyl)-1-[3-(trifluoromethyl)benzyl]-indole-2-carboxylic acid ethyl ester (12.9 mg, 22 nmol; see Example 19(c)), phenylboronic acid (4.1 mg, 33 nmol), K₃PO₄ (17 mg, 78 nmol), Pd(OAc)₂ (0.25 mg, 1.0 nmol) and toluene (500 μL) was heated at 170° C. for 5 minutes using microwave irradiation. The mixture was filtered through Celite® and the filter cake was washed with Et₂O. The combined filtrates were poured into NaHCO₃ (aq., sat.) and extracted with Et₂O. The combined extracts were dried over Na₂SO₄ and concentrated. Purification by chromatography yielded the sub-title compound (60%).

### (b) 5-(4-Methoxyphenyl)-3-phenyl-1-[3-(trifluoromethyl)benzyl]indole-2-carboxylic acid

The title compound was prepared from 5-(4-methoxyphenyl)-3-phenyl-1-[3-(trifluoromethyl)benzyl]indole-2-carboxylic acid ethyl ester (see step (a)) in accordance with Example 19(e).

### Example 21

### 3,5-Bis(4-methoxyphenyl)-1-[3-(trifluoromethyl)benzyl]indole-2-carboxylic acid

The title compound was prepared in accordance with Example 20 using 4-methoxyphenylboronic acid instead of phenylboronic acid.

¹H NMR (200 MHz, CDCl₃): δ 7.68 (m, 1H), 7.59 (dd, J=9, 2 Hz, 1H), 7.52-7.46 (m, 5H), 7.45 (m, 1H), 7.40-7.34 (m, 2H), 7.19 (d, J=7 Hz, 1H), 7.02 (m, 2H), 6.95 (m, 2H), 5.87 (s, 2H), 3.89 (s, 3H), 3.84 (s, 3H).

### Example 22

### 5-(4-Methoxyphenyl)-3-(3-nitrophenyl)-1-[3-(trifluoromethyl)benzyl]-indole-2-carboxylic acid

The title compound was prepared in accordance with Example 19 using 3-nitrophenylboronic acid and 2.5 mol % Pd(PPh₃)₂Cl₂.

¹H NMR (200 MHz, CDCl₃): δ 8.39 (m, 1H), 8.26 (m, 1H), 7.83 (m, 1H), 7.65 (m, 1H), 7.62 (m, 1H), 7.58 (m, 1H), 7.54-7.34 (m, 6H), 7.19 (d, J=7 Hz, 1H), 6.95 (m, 2H), 5.89 (s, 2H), 3.83 (s, 3H).

### Example 23

### 5-(4-Methoxyphenyl)-3-(pyridin-3-yl)-1-[3-(trifluoromethyl)benzyl]indole-2-carboxylic acid

The title compound was prepared in accordance with Example 19 using pyridin-3-ylboronic acid and 5 mol % Pd(PPh₃)₂Cl₂.

¹H NMR (200 MHz, DMSO-d₆): δ 8.76 (s, 1H), 8.50 (d, J=4 Hz, 1H), 8,00 (d, J=8 Hz, 1H), 7.72 (s, 1H), 7.63-7.37 (m, 9H), 6.98 (m, 2H), 5.95 (s, 2H), 3.77 (s, 3H).

### Example 24

### 3-(4-Fluoro-3-methylphenyl)-5-(4-methoxyphenyl)-1-[3-(trifluoromethyl)-benzyl]indole-2-carboxylic acid

The title compound was prepared in accordance with Example 19 using 4-fluoro-3-methylphenylboronic acid and 5 mol % Pd(PPh₃)₂Cl₂.

¹H NMR (200 MHz, DMSO-d₆): δ 7.69-7.50 (m, 8H), 7.42 (m, 1H), 7.37-7.28 (m, 2H), 7.24-7.17 (m, 1H), 6.98 (m, 2H), 5.94 (s, 2H), 3.76 (s, 3H), 2.29 (d, J=2 Hz, 3H).

### Example 25

### 3-(3,5-Dichlorophenyl)-5-(4-methoxyphenyl)-1-[3-(trifluoromethyl)-benzyl]indole-2-carboxylic acid

The title compound was prepared in accordance with Example 19 using 3,5-dichlorophenylboronic acid and 3.5 mol % Pd(PPh₃)₂Cl₂.

¹H NMR (200 MHz, CDCl₃): δ 7.63-7.58 (m, 2H), 7.53-7.44 (m, 4H), 7.40-7.34 (m, 5H), 7.14 (d, J=8 Hz, 1H), 6.97 (m, 2H), 5.82 (s, 2H), 3.84 (s, 3H).

### Example 26

### 5-(2-Methoxyphenyl)-3-(3-nitrophenyl)-1-[3-(trifluoromethyl)benzyl]-indole-2-carboxylic acid

### (a) 5-(2-Methoxyphenyl)indole-2-carboxylic acid ethyl ester

A mixture of 5-bromoindole-2-carboxylic acid ethyl ester (268 mg, 1 mmol), 2-methoxyphenylboronic acid (304 mg, 2 mmol), Pd(PPh₃)₂Cl₂ (35 mg, 50 mmol), Na₂CO₃ (159 mg, 1.5 mmol), and DME/H₂O/EtOH 7:3:2 (3 mL) was heated at 160° C. for 8 minutes using microwave irradiation. The mixture was poured into water and extracted with EOAc. The combined extracts were dried over Na₂SO₄ and concentrated. The residue was crystallised from EtOH/H₂O to yield the sub-title compound (37%).

### (b) 5-(2-Methoxyphenyl)-3-(3-nitrophenyl)-1-[3-(trifluoromethyl)benzyl]-indole-2-carboxylic acid

The title compound was prepared in accordance with Example 19(b), 19(c) and 19(d) using 3-nitrophenylboronic acid and 2.5 mol % Pd(PPh₃)₂Cl₂.

¹H NMR (200 MHz, DMSO-d₆): δ 13.31 (br s, 1H), 8.32 (m, 1H), 8.23 (m, 1H), 7.98 (d, J=8 Hz, 1H), 7.80-7.49 (m, 7H), 7.32-7.26 (m, 3H), 7.07 (d, J=8 Hz, 1H), 6.98 (t, J=8 Hz, 1H), 6.01 (s, 2H), 3.72 (s, 3H).

### Example 27

### 5-(2-Methoxyphenyl)-3-(4-methoxyphenyl)-1-[3-(trifluoromethyl)benzyl]-indole-2-carboxylic acid

The title compound was prepared in accordance with Example 26 using 4-methoxyphenylboronic acid and 2.5 mol % Pd(PPh₃)₂Cl₂.

¹H NMR (200 MHz, DMSO-d₆): δ 12.98 (br s, 1H), 7.66-7.60 (m, 3H), 7.55 (t, J=8 Hz, 1H), 7.50-7.38 (m, 4H), 7.34-7.24 (m, 3H), 7.06 (d, J=8 Hz, 1H), 7.04-6.95 (m, 3H), 5.95 (s, 2H), 3.81 (s, 3H), 3.71 (s, 3H).

### Example 28

### 3-(3,5-Dichlorophenyl)-5-(2-methoxyphenyl)-1-[3-(trifluoromethyl)benzyl]indole-2-carboxylic acid

The title compound was prepared in accordance with Example 26 using 3,5-dichlorophenylboronic acid and 2.5 mol % Pd(PPh₃)₂Cl₂.

¹H NMR (200 MHz, DMSO-d₆): δ 7.72 (s, 1H), 7.65-7.56 (m, 5H), 7.54-7.51 (m, 2H), 7.46 (m, 1H), 7.35-7.27 (m, 3H), 7.10 (m, 1H), 6.99 (m, 1H), 5.96 (s, 2H), 3.73 (s, 3H).

### Example 29

### 5-(3-Acetylphenyl)-3-(4-methoxyphenyl)-1-[3-(trifluoromethyl)benzyl]-indole-2-carboxylic acid

### (a) 5-(3-Acetylphenyl)indole-2-carboxylic acid ethyl ester

The sub-title compound was prepared in accordance with Example 26(a) using 3-acetylphenylboronic acid and 2.5 mol % Pd(PPh₃)₂Cl₂ and heating at 160° C. for 10 min.

### (b) 5-(3-Acetylphenyl)-3-(4-methoxyphenyl)-1-[3-(trifluoromethyl)benzyl]-indole-2-carboxylic acid

The title compound was prepared in accordance with Example 26(b) using 4-methoxyphenylboronic acid and 2.5 mol % Pd(PPh₃)₂Cl₂.

¹H NMR (200 MHz, DMSO-d₆): δ 13.06 (br s, 1H), 8.10 (s, 1H), 7.89 (m, 2H), 7.80-7.67 (m, 3H), 7.65-7.54 (m, 4H), 7.44 (m, 2H), 7.28 (d, J=8 Hz, 1H), 7.03 (m, 2H), 5.98 (s, 2H), 3.82 (s, 3H), 2.63 (s, 3H).

### Example 30

### 3,5-Bis(3-acetylphenyl)-1-[3-(trifluoromethyl)benzyl]indole-2-carboxylic acid

The title compound was prepared in accordance with Example 29 using 3-acetylphenylboronic acid and 2.5 mol % Pd(PPh₃)₂Cl₂

¹H NMR (200 MHz, DMSO-d₆): δ 13.21 (br s, 1H), 8.10 (m, 2H), 7.98 (m, 1H), 7.89 (m, 2H), 7.84-7.78 (m, 2H), 7.74 (m, 1H), 7.70 (m, 1H), 7.67-7.52 (m, 5H), 7.30 (d, J=8 Hz, 1H), 6.02 (s, 2H), 2.64 (s, 3H), 2.62 (s, 3H).

### Example 31

### 5-(3-Acetylphenyl)-3-phenyl-1-[3-(trifluoromethyl)benzyl]indole-2-carboxylic acid

The title compound was prepared in accordance with Example 29 using phenylboronic acid and 2.5 mol % Pd(PPh₃)₂Cl₂.

¹H NMR (200 MHz, DMSO-d₆): δ 13.14 (br s, 1H), 8.10 (m, M1), 7.89 (m, 2H), 7.82-7.67 (m, 3H), 7.65-7.44 (m, 8H), 7.38 (m, 1H), 7.30 (d, J-Hz, 1H), 5.99 (s, 2H), 2.62 (s, 3H).

### Example 32

### 5-(3-Acetylphenyl)-3-(4-pyridyl)-1-[3-(trifluoromethylbenzyl]indole-2-carboxylic acid

The title compound was prepared in accordance with Example 29 using 4-pyridylboronic acid and 2.5 mol % Pd(PPh₃)₂Cl₂.

¹H NMR (200 MHz, DMSO-d₆): δ 8.65 (m, 2H), 8.13 (m, 1H), 7.91 (m, 2H), 7.85-7.80 (m, 1H), 7.77-7.73 (m, 2H), 7.66-7.51 (m, 6H), 7.30 (d, J=8 Hz, 1H), 6.01 (s, 2H), 2.63 (s, 3H).

### Example 33

### 5-(3-Acetylphenyl)-3-(4-chlorophenyl)-1-[3-(trifluoromethyl)benzyl]indole-2-carboxylic acid

The title compound was prepared in accordance with Example 29 using chlorophenylboronic acid and 2.5 mol % Pd(PPh₃)₂Cl₂.

¹H NMR (200 MHz, DMSO-d₆): δ 13.22 (br s, 1H), 8.12 (m, 1H), 7.90 (m, 2H), 7.83-7.67 (m, 3H), 7.65-7.50 (m, 8H), 7.29 (d, J=8 Hz, 1H), 6.00 (s, 2H), 2.63 (s, 3H).

### Example 34

### 5-(4-Chlorophenyl-3-(3-pyridyl)-1-[3-(trifluoromethyl)benzyl]indole-2-carboxylic acid

### (a) 5-(4-Chlorophenyl)indole-2-carboxylic acid ethyl ester

The sub-title compound was prepared in accordance with Example 26(a) using 4-chlorophenylboronic acid and 4.0 mol % Pd(PPh₃)₂Cl₂ and heating at 160° C. for 10 min.

### (b) 1-(3-(Trifluoromethyl)benzyl)-5-(4-chlorophenyl)-3-(3-pyridyl)-1-[3-(trifluoromethyl)benzyl]indole-2-carboxylic acid

The title compound was prepared in accordance with Example 26(b) using 3-pyridylboronic acid and 2.5 mol % Pd(PPh₃)₂Cl₂.

### Example 35

### 5-(4-Chlorophenyl)-3-(4-(hydroxymethyl)phenyl)-1-[3-(trifluoromethyl)benzyl]indole-2-carboxylic acid

The title compound was prepared in accordance with Example 34 using 4-(hydroxymethyl)phenylboronic acid and 5 mol % Pd(PPh₃)₂Cl₂.

¹H NMR (200 MHz, DMSO-d₆): δ 7.74 (m, 2H), 7.66-7.58 (m, 4H), 7.55-7.26 (m, 9H), 5.95 (s, 2H), 5.29 (br s, 1H), 4.57 (s, 2H).

### Example 36

### 5-(4-Chlorophenyl)-3-(4-fluoro-3-methylphenyl)-1-[3-(trifluoromethyl)benzyl]indole-2-carboxylic acid

The title compound was prepared in accordance with Example 34 using 4-fluoro-3-methylphenylboronic acid and 5 mol % Pd(PPh₃)₂Cl₂.

### Example 37

### 5-(4-Chlorophenyl)-3-(4-ethylphenyl)-1-[3-(trifluoromethyl)benzyl]indole-2-carboxylic acid

The title compound was prepared in accordance with Example 34 using 4-ethylphenylboronic acid and 5 mol % Pd(PPh₃)₂Cl₂.

¹H NMR (200 MHz, DMSO-d₆): δ 7.76-7.70 (m, 1H), 7.66-7.58 (m, 5H), 7.57-7.50 (m, 2H), 7.48-7.40 (m, 4H), 7.33-7.30 (m, 3H), 5.94 (s, 2H), 2.68 (q, J=8 Hz, 21), 1.25 (t, J=8 Hz, 3H).

### Example 38

### 6-(3-Aminophenyl)-3-phenyl-1-[(3-(trifluoromethoxy)benzyl]indole-2-carboxylic acid hydrochloride

### (a) 6-(3-Aminophenyl-3-phenyl-1-[(3-(trifluoromethoxy)benzyl]indole-2-carboxylic acid ethyl ester hydrochloride

6-(3-Nitrophenyl)-3-phenyl-1-[(3-(trifluoromethoxy)benzyl]-indole-2-carboxylic acid ethyl ester, prepared in accordance with the procedure in Example 1(a)-(d) from 6-bromoindole-2-carboxylic acid ethyl ester, 3-nitrophenylboronic acid and 3-(trifluoromethoxy)benzyl chloride (1.55 g, 2.76 mmol), in EtOAc (35 mL) was hydrogenated at ambient temperature and pressure over Pd—C (10%, 440 mg) until all starting material was consumed as judged by TLC. The mixture was filtered through Celite® and the filtrate concentrated. The residue was purified by chromatography, dissolved in anhydrous Et₂O, whereafter the sub-title compound was precipitated by the addition of an excess of HCl (4M) in dioxane. Yield: 955 mg (73%).

### (b) 6-(3-Aminophenyl)-3-phenyl-1-[(3-(trifluoromethoxy)benzyl]indole-2-carboxylic acid hydrochloride

The title compound was prepared by hydrolysis of 6-(3-aminophenyl)-3-phenyl-1-[(3-(trifluoromethoxy)benzyl]-indole-2-carboxylic acid ethyl ester in accordance with the procedure described in Example 2(b) (2M KOH (aq.), dioxane, 100° C., 1 h), followed by precipitation from an ethereal solution by addition of HCl (4M) in dioxane as described above,

¹H NMR (200 MHz, DMSO-d₆): δ 11.5-9.6 (3H, br s), 7.94 (1H, s), 7.67-7.05 (15H, m), 5.99 (2H, s), 4.5-3.0 (1H, br s).

### Example 39

### 6-[3-(2,2-Dimethylpropionylamino)phenyl]-3-phenyl-1-[3-(trifluoromethoxybenzyl]indole-2-carboxylic acid

### (a) 6-[3-(2,2-Dimethylpropionylamino)phenyl]-3-phenyl-1-[3-(trifluoromethoxy)benzyl]indole-2-carboxylic acid ethyl ester

Pivaloyl chloride (156 μL, 1.27 mmol) was added over 5 min to a stirred solution of 6-(3-aminophenyl)-3-phenyl-1-[3-(trifluoromethoxy)benzyl]-indole-2-carboxylic acid ethyl ester hydrochloride (600 mg, 1.06 mmol; see Example 38(a)), DMAP (65 mg, 0.53 mmol), Et₃N (530 μL, 3.8 mmol), and dry CH₂Cl₂ (10 mL). The mixture was stirred at room temperature overnight whereafter another portion of pivaloyl chloride (0.156 μL, 1.27 mmol) and Et₃N (530, μL, 3.8 mmol) was added. After 2 h at room temperature, the mixture was diluted with CH₂Cl₂ and washed with HCl (aq., 1M), NaHCO₃ (aq., sat.) and brine, and concentrated. The residue was treated with pentane to give the title compound as a white solid (480 mg, 74%).

### (b) 6-[3-(2,2-Dimethylpropionylamino)phenyl]-3-phenyl-1-[3-(trifluoromethoxy)benzyl]indole-2-carboxylic acid

The title compound was prepared by hydrolysis of 6-[3-(2,2-dimethylpropionylamino)phenyl]-3-phenyl-1-[3-(trifluoromethoxy)benzyl]indole-2-carboxylic acid ethyl ester in accordance with the procedure described in Example 1(e) (2M KOH (aq.), dioxane, 120° C., 2 h).

¹H NMR (200 MHz, DMSO-d₆): δ 13.0 (1H, s), 9.29 (1H, s), 7.98-7.96 (1H, m), 7.90 (1H, s), 7.72-7.66 (1H, m), 7.59-7.36 (10H, m), 7.26-7.19 (2H, m), 7.11-7.07 (1H, m), 6.00 (2H, s), 1.24 (9H, s).

### Example 40

### 6-(3-(Methanesulfonylamino)phenyl)-3-phenyl-1-[3-(trifluoromethoxy)benzyl]indole-2-carboxylic acid

The title compound was prepared in accordance with the procedure in Example 39 using methanesulfonyl chloride instead of pivaloyl chloride.

¹H NMR (200 MHz, DMSO-d₆): δ 13.2-12.9 (1H, br s), 9.80 (1H, s), 7.88-7.86 (1H, m), 7.56-7.35 (1H, m), 7.24-7.15 (3H, m), 7.10-7.05 (1H, m), 5.97 (2H, s), 3.00 (3H, s).

### Example 41

### 6-(3-But-3-enylaminophenyl)-3-phenyl-1-[(3-(trifluoromethoxy)benzyl]-indole-2-carboxylic acid

### (a) 6-(3-But-3-enylaminophenyl)-3-phenyl-1-[(3-(trifluoromethoxy)benzyl]indole-2-carboxylic acid ethyl ester

4-Bromo-1-butene (143 mg, 1.06 mmol) was added to a mixture of 6-(3-aminophenyl)-3-phenyl-1-[3-(trifluoromethoxy)benzyl]indole-2-carboxylic acid ethyl ester hydrochloride (400 mg, 0.71 mmol; see Example 38(a)), NaI (317 mg, 2.12 mmol), K₂CO₃ (390 mg, 2.84 mmol), and dry DMF (3 mL). The mixture was heated at 100° C. for 12 h, allowed to cool and poured into water. The mixture was extracted with EtOAc and the combined extracts were washed with water, brine, dried over Na₂CO₃ and concentrated. The residue was purified by chromatography to give the title compound (173 mg, 42%).

### (b) 6-(3-But-3-enylaminophenyl)-3-phenyl-1-[(3-(trifluoromethoxy)benzyl]indole-2-carboxylic acid

The title compound was prepared by hydrolysis of 6-(3-but-3-enylaminophenyl)-3-phenyl-1-[(3-(trifluoromethoxy)benzyl]indole-2-carboxylic acid ethyl ester in accordance with the procedure described in Example 1(e) (NaOH (aq., 40%), DMF, room temperature, 2 h).

¹H NMR (200 MHz, DMSO-d₆): δ 7.79 (1H, s), 7.51-7.31 (8H, m), 7.24-7.06 (4H, m), 6.84-6.80 (2H, m), 6.57-6.52 (1H, m), 5.97 (2H, s), 5.88 (1H, ddt, J=17.2, 10.3, 6.7 Hz), 5.15-4.98 (2H, m), 3.11 (2H, t, J=7.2 Hz) 2.30 (2H, q, J=6.9 Hz).

### Example 42

### 6-[3-(Allylmethanesulfonylamino)phenyl]-3-phenyl-1-(3-trifluoromethoxybenzyl)-indole-2-carboxylic acid

Allyl iodide (54 μL, 0.59 mmol) was added to a mixture of 6-[3-(methanesulfonylamino)phenyl]-3-phenyl-1-(3-trifluoromethoxy-benzyl)indole-2-carboxylic acid ethyl ester (178 mg, 0.29 mmol; see Example 40), Cs₂CO₃ (333 mg, 1.02 mmol), and dry DMF (2.5 mL). The mixture was heated at 85° C. for 30 min using microwave irradiation, allowed to cool and poured into water. The mixture was extracted with EtOAc and the combined extracts were washed with brine, dried over Na₂CO₃ and concentrated. The residue was hydrolyzed in accordance with the procedure described in Example 2(b) (2M KOH (aq.), dioxane, 110° C., 55 min) to give the title compound (140 mg, 78%).

¹H NMR (200 MHz, DMSO-d₆): δ 13.08 (1H, s), 7.94 (1H, s), 7.68-7.63 (2H, m), 7.58-7.34 (10H, m), 7.25-7.20 (2H, m), 7.12-7.07 (1H, m), 6.01 (2H, s), 5.79 (1H, ddt, J=17.1, 10.2, 5.9 Hz), 5.19 (1H, dd, J=17.1, 1.5 Hz), 5.07 (1H, dd, J=10.2, 1.5 Hz), 4.35 (2H, d, J=5.9 Hz), 3.05 (3H, s).

### Example 43

### 1-(3-Chlorobenzyl)-6-(3,5-difluorophenyl)-3-[3-(4-methoxyphenyl)-propionylamino]indole-2-carboxylic acid

### (a) 3-Amino-1-(3-chlorobenzyl)-6-(3,5-difluorophenyl)indole-2-carboxylic acid ethyl ester

A mixture of 3-amino-6-bromo-1-(3-chlorobenzyl)indole-2-carboxylic acid ethyl ester (1.04 g, 2.25 mmol, see Example 54(b)) Pd(OAc)₂ (31 mg, 0.14 mmol), tri-o-tolylphosphine (84 mg, 0.28 mmol), K₂CO₃ (1.33 g, 9.64 mmol) and toluene (30 mL) was stirred under argon at room temperature for 10 min whereafter 3,5-dimethoxyphenylboronic acid (0.78 g, 4.13 mmol) and EtOH (10 mL) was added. The mixture was heated at reflux for 2.5 h allowed to cool and filtered through Celite®. The filter cake was washed with EtOAc and the combined filtrates were washed with NaHCO₃ (aq., sat.). The aqueous phase was extracted with EtOAc and the combined organic phases were washed with water, brine and dried over Na₂CO₃. Concentration and purification by chromatography gave the title compound (1.28 g, 98%).

### (b) 1-(3-Chlorobenzyl)-6-(3,5-difluorophenyl)-3-[3-(4-methoxyphenyl)-propionylamino]indole-2-carboxylic acid ethyl ester

DMAP (22.3 mg, 0.18 mmol) and Et₃N (154 μL, 1.1 mmol) were added at room temperature to a stirred mixture of 3-amino-1-(3-chlorobenzyl)-6-(3,5-difluorophenyl)indole-2-carboxylic acid ethyl ester (150 mg, 0.37 mmol), 3-(4-methoxyphenyl)propionyl chloride (109 mg, 0.55 mmol) and MeCN (3.5 mL). The mixture was stirred at room temperature overnight, poured into HCl (aq., 1M) and extracted with EtOAc. The combined extracts were washed with NaHCO₃ (aq., sat.), dried over Na₂CO₃ and concentrated. The sub-title compound was obtained by crystallisation of the residue from EtOAc/benzene (108 mg, 50%).

### (c) 1-(3-Chlorobenzyl)-6-(3,5-difluorophenyl-3-[3-(4-methoxyphenyl)-propionylamino]indole-2-carboxylic acid

The title compound was prepared by hydrolysis of 1-(3-chlorobenzyl)-6-(3,5-difluorophenyl)-3-[3-(4-methoxyphenyl)-propionylamino]indole-2-carboxylic acid ethyl ester (108 mg, 0.18 mmol) according to the procedure described in Example 2(b) (KOH (aq., 2M), dioxane, 60° C. 50 min then 100° C. 30 min). Yield 75 mg (73%).

¹H NMR (200 MHz, DMSO-d₆): δ 13.6-13.2 (1H, br s), 9.73 (1H, s), 8.09-8.03 (1H, m), 7.57-6.86 (13H, m), 5.94 (2H, s), 3.73 (3H, s), 2.95-2.86 (2H, m), 2.73-2.64 (2H, m).

The following Examples 44 to 53 were made in accordance with the procedure in Example 43 using the appropriate acid chloride or sulfonyl chloride.

### Example 44

### 1-(3-Chlorobenzyl)-6-(3,5-difluorophenyl)-3-[(3,5-dimethyladamantane-1-carbonyl)amino]indole-2-carboxylic acid

¹H NMR (200 MHz, DMSO-d₆): δ 13.8-13.2 (1H, br s), 9.52 (1H, s), 8.04 (1H, s), 7.81 (1H, d, J=8.6 Hz), 7.60-7.48 (3H, m), 7.34-7.16 (3H, m), 7.11-7.10 (1H, m), 6.98-6.90 (1H, m), 5.94 (2H, s), 2.16-2.11 (1H, m), 1.81-1.80 (2H, m), 1.68-1.53 (4H, m), 1.45-1.30 (4H, m), 1.23-1.14 (2H, m), 0.87 (6H, s).

### Example 45

### 3-(2-Adamant-1-ylacetylamino)-1-(3-chlorobenzyl)-6-(3,5-difluorophenyl)indole-2-carboxylic acid

¹H NMR (200 MHz, DMSO-d₆): δ 13.5-13.2 (1H, br s), 9.56 (1H, s), 8.04 (1H, s), 7.70 (1H, d, J=8.6 Hz), 7.59-7.49 (3H, m), 7.33-7.07 (4H, m), 6.94-6.89 (1H, m), 5.90 (2H, s), 2.14 (2H, s), 1.96-1.92 (3H, m), 1.69-1.63 (12H, m).

### Example 46

### 1-(3-Chlorobenzyl)-6-(3,5-difluorophenyl)-3-(toluene-4-sulfonylamino)indole-2-carboxylic acid

¹H NMR (200 MHz, DMSO-d₆): δ 13.8-12.8 (1H, br s), 9.33 (1H, s), 8.02 (1H, s), 7.78 (1H, d, J=8.6 Hz), 7.58-7.52 (3H, m), 7.44-7.38 (2H, m), 7.34-7.17 (4H, m), 6.99 (2H, s), 6.79-6.73 (1H, m), 5.84 (2H, s), 2.33 (3H, s).

### Example 47

### 1-(3-Chlorobenzyl)-3-(2-cyclohexylideneacetylamino)-6-(3,5-difluorophenyl)indole-2-carboxylic acid

¹H NMR (200 MHz, DMSO-d₆): δ 13.7-12.8 (1H, br s), 9.62 (1H, s), 8.03 (1H, s), 7.74 (1H, d, J=8.6 Hz), 7.58-7.48 (3H, m), 7.33-7.23 (2H, m), 7.18 (1H, ddd, J=9.3, 2.3, 2.3 Hz), 7.09-7.07 (1H, m), 6.97-6.89 (1H, m), 5.94 (1H, s), 5.91 (2H, s), 2.89-2.83 (2H, m), 2.21-2.16 (2H, m), 1.65-1.52 (6H, m).

### Example 48

### 1-(3-Chlorobenzyl)-6-(3,5-difluorophenyl)-3-[2-(3,3,5,5-tetramethylcyclohexylidene)acetylamino]indole-2-carboxylic acid

¹H NMR (200 MHz, DMSO-d₆): δ 13.6-13.1 (1H, br s), 9.67 (1H, s), 8.06 (1H, s), 7.74 (1H, d, J=8.6 Hz), 7.61-7.51 (3H, m), 7.35-7.16 (3H, m), 7.11-7.09 (1H, m), 6.99-6.91 (1H, m), 6.07 (1H, s), 5.94 (2H, s), 2.70 (2H, s), 1.99 (2H, s), 1.33 (2H, s), 0.97-0.96 (12H, m).

### Example 49

### 1-(3-Chlorobenzyl)-6-(3,5-difluorophenyl)-3-[2-(3,3,5,5-tetramethylcyclohexylidene)acetylamino]indole-2-carboxylic acid sodium salt

¹H NMR (200 MHz, DMSO-d₆): δ 12.13 (1H, s), 8.38 (1H, d, J=8.6 Hz), 7.76 (1H, s), 7.48-7.42 (2H, m), 7.36-7.29 (1H, m), 7.25-7.15 (3H, m), 7.11-7.04 (2H, m), 6.17 (2H, s), 5.85 (1H, s), 2.74 (2H, s), 1.97 (2H, s), 1.31 (2H, s), 0.97-0.92 (12H, m).

### Example 50

### 1-(3-Chlorobenzyl)-6-(3,5-difluorophenyl)-3-(4-isopropoxybenzoylamino)indole-2-carboxylic acid

¹H NMR (200 MHz, DMSO'd₆): δ 13.6-13.2 (1H, br s), 10.13 (1H, s), 8.08 (1H, s), 8.03-7.96 (2H, m), 7.83 (1H, d, J=8.6 Hz), 7.62-7.51 (3H, m), 7.35-7.12 (4H, m), 7.08-7.01 (2H, m), 6.99-6.93 (1H, m), 5.96 (2H, s), 4.74 (1H, septet, J=6.1 Hz), 1.29 (6H, d, J=6.1 Hz).

### Example 51

### 1-(3-Chlorobenzyl)-6-(3,5-difluorophenyl)-3-(4-isopropoxybenzoylamino)indole-2-carboxylic acid sodium salt

¹H NMR (200 MHz, DMSO-d₆): δ 13.14 (1H, br s), 8.49 (1H, d, J=8.4 Hz), 7.98-7.91 (2H, m), 7.81 (1H, s), 7.51-7.44 (2H, m), 7.40-7.33 (1H, m), 7.25-7.17 (3H, m), 7.16-6.99 (4H, m), 6.19 (2H, s), 4.72 (1H, septet, J=6.0 Hz), 1.28 (6H, d, J=6.0 Hz).

### Example 52

### 1-(3-Chlorobenzyl)-6-(3,5-difluorophenyl)-3-[4-(trifluoromethyl)-benzoylamino]indole-2-carboxylic acid

¹H NMR (200 MHz, DMSO-d₆): δ 13.7-13.1 (1H, br s), 10.49 (1H, s), 8.23 (2H, m), 8.11 (1H, s), 7.93 (2H, m), 7.77 (1H, d, J-8.6 Hz), 7.59-7.53 (3H, m), 7.35-7.09 (4H, m), 6.99-6.94 (1H, m), 5.97 (2H, s).

### Example 53

### 1-(3-Chlorobenzyl)-3-[(6-chloropyridine-3-carbonyl)amino]-6-(3,5-difluorophenyl)indole-2-carboxylic acid

¹H NMR (200 MHz, DMSO-d₆): δ 14.21-14.16 (1H, br s), 8.99 (1H, d, J=2.2 Hz), 8.56 (1H, d, J=8.6 Hz), 8.37 (1H, dd, J=8.4, 2.6 Hz), 7.83-7.82 (1H, m), 7.74 (1H, d, J=8.2 Hz), 7.51-7.36 (3H, m), 7.30-7.06 (5H, m), 6.21 (2H, s).

### Example 54

### 3-(4-Chlorobenzoylamino)-1-(3-chlorobenzyl)-6-[4-(methylsulfonyl)-phenyl]indole-2-carboxylic acid

### (a) 6-Bromo-1-(3-chlorobenzyl)-3-nitroindole-2-carboxylic acid ethyl ester

Cu(NO₃)₂×2.5H₂O (2.26 g, 9.74 mmol) was added to Ac₂O (10 mL) at 5° C. The mixture was stirred until a homogenous solution was formed whereafter a solution 6-bromo-1-(3-chlorobenzyl)indole-2-carboxylic acid ethyl ester, prepared from 6-bromoindole-2-carboxylic acid ethyl ester and 3-chlorobenzyl chloride in accordance with the procedure in Example 1(c), (4.72 g, 12.02 mmol), in Ac₂O (20 mL) was added dropwise. The mixture was allowed to come to room temperature and was stirred for 1.5 h and filtered. The filtrate was poured onto ice and was left to stir overnight. The precipitate was collected and dried to give the sub-title compound (4.82 g, 92%).

### (b) 3-Amino-6-bromo-1-(3-chlorobenzyl)indole-2-carboxylic acid ethyl ester

A mixture of 6-bromo-1-(3-chlorobenzyl)-3-nitroindole-2-carboxylic acid ethyl ester (4.82 g, 11.01 mmol), Fe-powder (3.15 g, 56.3 mmol), NH₄Cl (aq., sat., 75 mL) and isopropanol (160 mL) was heated at reflux for 2 h, whereafter additional portions of Fe-powder (3.15 g, 56.3 mmol) and NH₄Cl (aq., sat., 75 mL) were added. The mixture was heated for an additional 2 h, allowed to cool and filtered through Celite®. The filter cake was washed with EtOAc and the combined filtrates were extracted with EtOAc. The combined extracts were washed with water, brine and dried over Na₂CO₃. Concentration and purification by chromatography gave the title compound (3.99 g, 89%).

### (c) 6-Bromo-3-(4-chlorobenzoylamino)-1-(3-chlorobenzyl)indole-2-carboxylic acid ethyl ester

A mixture of 3-amino-6-bromo-1-(3-chlorobenzyl)indole-2-carboxylic acid ethyl ester (2.00 g, 4.91 mmol), 4-chlorobenzoyl chloride (1.72 g, 9.82 mmol), DMAP (300 mg, 2.46 mmol), Et₃N (1.38 mL, 9.82 mmol) and MeCN (50 mL) was stirred at room temperature for 24 h. The mixture was poured into water and extracted with EtOAc. The combined extracts were washed with water, brine and dried over Na₂CO₃, Concentration, crystallisation from EtOH/EtOAc (1:1) and chromatography gave the title compound.

### (d) 3-(4-Chlorobenzoylamino)-1-(3-chlorobenzyl)-6-[4-(methylsulfonyl)-phenyl]indole-2-carboxylic acid ethyl ester

A mixture of 6-bromo-3-(4-chlorobenzoylamino)-1-(3-chlorobenzyl)indole-2-carboxylic acid ethyl ester (160 mg, 0.29 mmol), 4-(methanesulfonyl)phenylboronic acid (87.9 mg, 0.44 mmol), Pd(OAc)₂ (3.4 mg, 0.015 mmol), tri-o-tolylphosphine (8.8 mg, 0.029 mmol), K₃PO₄ (215 mg, 1.02 mmol), toluene (3 ml) and EtOH (0.5 mL) was stirred at room temperature for 20 min and heated at 90° C. for 3 h. The mixture was allowed to cool, poured into NaHCO₃ (aq., sat.) and extracted with EtOAc. The combined extracts were washed brine and dried over Na₂CO₃. Concentration and purification by chromatography gave the title compound (120 mg, 67%).

### (e) 3-(4-Chlorobenzoylamino)-1-(3-chlorobenzyl)-6-[4-(methylsulfonyl)-phenyl]indole-2-carboxylic acid

The title compound was prepared by hydrolysis of 3-(4-chlorobenzoylamino)-1-(3-chlorobenzyl)-6-[4-(methylsulfonyl)phenyl]indole-2-carboxylic acid ethyl ester (120 mg, 0.19 mmol) according to the procedure described in Example 1 (e). Yield 84 mg (74%).

¹H NMR (200 MHz, DMSO-d₆): δ 13.6-13.4 (1H, br s), 10.36 (1H, s), 8.12-7.97 (7H, m), 7.83 (1H, d, J=8.4 Hz), 7.69-7.61 (2H, m), 7.57 (1H, d, J=8.7 Hz), 7.38-7.25 (2H, m), 7.17-7.13 (1H, m), 7.03-6.96 (1H, m), 6.00 (2H, s), 3.26 (3H, s).

The following Examples 55 to 64 were made in accordance with the procedure in Example 54 using the appropriately substituted phenylboronic is acids.

### Example 55

### 3-(4-Chlorobenzoylamino)-1-(3-chlorobenzyl)-6-[4-(methylthio)phenyl]-indole-2-carboxylic acid

¹H NMR (200 MHz, DMSO-d₆): δ 13.7-13.1 (1H, br s), 10.35 (1H, s), 8.12-8.04 (2H, m), 7.93 (1H, s), 7.77 (1H, d, J=8.5 Hz), 7.74-7.61 (4H, m), 7.47 (1H, d, J=8.5 Hz), 7.39-7.25 (4H, m), 7.16-7.12 (1H, m), 7.04-6.97 (1H, m), 5.97 (2H, s), 2.51 (3H, s).

### Example 56

### 3-(4-Chlorobenzoylamino)-1-(3-chlorobenzyl)-6-(4-vinylphenyl)indole-2-carboxylic acid

¹HNMR (200 MHz, DMSO-d₆): δ 13.6-13.2 (1H, br s), 10.35 (1H, s), 8.12-8.04 (2H, m), 7.97 (1H, s), 7.78 (1H, d, J=8.4 Hz), 7.78-7.72 (2H, m), 7.69-7.62 (2H, m), 7.61-7.54 (2H, m), 7.51 (1H, d, J=8.6 Hz), 7.38-7.25 (2H, m), 7.18-7.13 (1H, m), 7.05-6.98 (1H, m), 6.79 (H, dd, J=17.8, 11.0 Hz), 5.98 (2H, s), 5.90 (1H, d, J=17.8 Hz), 5.29 (1H, d, 11.0 Hz).

### Example 57

### 3-(4-Chlorobenzoylamino)-1-(3-chlorobenzyl)-6-(4-isopropoxyphenyl)indole-2-carboxylic acid

¹H NMR (200 MHz, DMSO-d₆): δ 13.5-13.3 (1H, br s), 10.35 (1H, s), 8.12-8.03 (2H, m), 7.85 (1H, s), 7.75 (1H, d, J=8.5 Hz), 7.70-7.61 (4H, m), 7.43 (1H, d, J=8.7 Hz), 7.38-7.25 (2H, m), 7.15-7.11 (1H, m), 7.05-6.96 (3H, m), 5.96 (2H, s), 4.67 (1H, septet, J=6.0 Hz), 1.28 (6H, d, J=6.0 Hz).

### Example 58

### 6-(4-tert-Butylphenyl)-3-(4-chlorobenzoylamino)-1-(3-chlorobenzyl)indole-2-carboxylic acid

¹HNMR (200 MHz, DMSO-d₆): δ 13.5-13.3 (1H, br s), 10.34 (1H, s), 8.12-8.03 (2H, m), 7.88 (1H, s), 7.76 (1H, d, J=8.4 Hz), 7.70-7.60 (4H, m), 7.52-7.42 (3H, m), 7.38-7.25 (2H, m), 7.17-7.13 (1H, m), 7.05-6.98 (1H, m), 5.96 (2H, s), 1.31 (9H, s).

### Example 59

### 3-(4-Chlorobenzoylamino)-1-(3-chlorobenzyl)-6-[4-(trifluoromethyl)phenyl]indole-2-carboxylic acid

¹H NMR (200 MHz, DMSO-d₆): δ 13.6-13.3 (1H, br s), 10.43 (1H, s), 8.12-8.04 (3H, m), 8.03-7.95 (2H, m), 7.87-7.78 (3H, m), 7.69-7.61 (2H, m), 7.54 (1H, dd, J=8.5, 1.0 Hz), 7.38-7.25 (2H, m), 7.16-7.12 (1H, m), 7.03-6.96 (1H, m), 6.00 (2H, s).

### Example 60

### 3-(4-Chlorobenzoylamino)-1-(3-chlorobenzyl)-6-[4-(trifluoromethoxy)-phenyl]indole-2-carboxylic acid

¹H NMR (200 MHz, DMSO-d₆): δ 13.6-13.2 (1H, br s), 10.39 (1H, s), 8.10-8.02 (2H, m), 7.97 (1H, s), 7.89-7.82 (2H, m), 7.79 (1H, d, J=8.5 Hz), 7.67-7.59 (2H, m), 7.50-7.41 (3H, m), 7.36-7.23 (2H, m), 7.14-7.10 (1H, m), 7.02-6.95 (1H, m), 5.96 (2H, s).

### Example 61

### 3-(4-Chlorobenzoylamino)-1-(3-chlorobenzyl)-6-(4-cyclohexylphenyl)-indole-2-carboxylic acid

¹H NMR (200 MHz, acetone-d₆): δ 11.9-11.5 (1H, br s), 8.61-8.52 (1H, m), 8.24-8.15 (2H, m), 7.71 (1H, s), 7.66-7.58 (2H, m), 7.58-7.49 (2H, m), 7.45-7.37 (1H, m), 7.34-7.12 (6H, m), 6.16 (2H, s), 2.64-2.47 (1H, m), 1.93-1.68 (5H, m), 1.60-1.22 (5H, m).

### Example 62

### 6-(4-Butylphenyl)-3-(4-chlorobenzoylamino)-1-(3-chlorobenzyl)indole-2-carboxylic acid

¹H NMR (200 MHz, DMSO-d₆): δ 13.5-13.2 (1H, br s), 8.49 (1H, d, J=8.6 Hz), 8.12-8.00 (2H, m), 7.70-7.59 (4H, m), 7.57 (1H, s), 7.37-7.17 (6H, m), 7.16-7.08 (1H, m), 6.20 (2H, s), 2.61 (2H, t, J=7.4 Hz), 1.67-1.49 (2H, m), 1.42-1.22 (2H, m), 0.91 (3H, t, J=7.2 Hz).

### Example 63

### 3-(4-Chlorobenzoylamino)-1-(3-chlorobenzyl)-6-(4-cyanophenyl)indole-2-carboxylic acid

¹H NMR (200 MHz, DMSO-d₆): δ 13.3-13.1 (1H, br s), 8.52 (1H, d, J=8.6 Hz), 8.10-8.01 (2H, m), 7.98-7.84 (5H, m), 7.68-7.60 (2H, m), 7.43 (1H, dd, J=8.6, 0.9 Hz), 7.34-7.18 (3H, m), 7.14-7.07 (1H, m), 6.21 (2H, s).

### Example 64

### 3-(4-Chlorobenzoylamino-1-(3-chlorobenzyl)-6-(4-chlorophenyl)indole-2-carboxylic acid

¹H NMR (200 MHz, DMSO-d₆): δ 13.8-13.2 (1H, br s), 11.8-11.4 (1H, br s), 8.14-8.02 (3H, m), 7.87 (1H, s), 7.81-7.73 (2H, m), 7.68-7.60 (2H, m), 7.56-7.48 (2H, m), 7.42 (1H, dd, J=8.7, 1.1 Hz), 7.36-7.22 (2H, m), 7.19-7.14 (1H, m), 7.08-7.00 (1H, m), 6.07 (2H, s).

The following Examples 65 to 109 were prepared by analogous techniques to those described herein.

### Example 65

### 1-(3-Chlorobenzyl)-6-(3,5-difluorophenyl)-3-(4-methoxybenzoylamino)indole-2-carboxylic acid

¹H NMR (200 MHz, DMSO-d₆): δ 10.19 (1H, s), 8.14-8.02 (3H, m), 7.86 (1H, d, J=8.6 Hz), 7.64-7.53 (3H, m), 7.39-7.07 (6H, m), 7.03-6.96 (1H, m), 6.00 (2H, s), 3.87 (3H, s).

### Example 66

### 1-(3-Chlorobenzyl)-3-(4-methoxybenzoylamino)-6-naphth-1-ylindole-2-carboxylic acid

¹H NMR (200 MHz, DMSO-d₆): δ 10.21 (1H, s), 8.09-7.91 (4H, m), 7.86 (1H, d, J=8.4 Hz), 7.66-7.69 (2H, m), 7.62-7.51 (2H, m), 7.48 (1H, d, J=1.2 Hz), 7.46-7.29 (3H, m), 7.23 (1H, dd, J=8.4, 1.2 Hz), 7.17-7.14 (1H, m), 7.13-7.05 (2H, m), 7.04-6.97 (1H, m) 5.91 (2H, s), 3.84 (3H, s).

### Example 67

### 6-Benzo[1,3]dioxol-5-yl-1-(3-cyanobenzyl)-3-(3-phenylpropionylamino)-indole-2-carboxylic acid

¹H NMR (200 MHz, DMSO-d₆): δ 9.7 (1H, s), 7.79 (1H, s), 7.70-7.64 (1H, m), 7.56-7.14 (12H, m), 6.97 (1H, d, J=8.1 Hz), 6.03 (2H, s), 5.92 (2H, s), 3.00-2.90 (2H, m), 2.75-2.64 (2H, m).

### Example 68

### 1-(3-Chlorobenzyl)-6-naphth-1-yl-3-pentanoylaminoindole-2-carboxylic acid

¹H NMR (200 MHz, DMSO-d₆): δ 13.37 (1H, s), 9.67 (1H, s), 8.02-7.91 (2H, m), 7.74-7.65 (3H, m), 7.61-7.25 (6H, m), 7.22 (1H, dd, J=8.4, 0.9 Hz), 7.12-7.09 (1H, m), 7.02-6.93 (1H, m), 5.86 (2H, s), 2.41 (2H, t, J=7.2 Hz), 1.72-1.56 (2H, m), 1.49-1.29 (2H, m), 0.93 (3H, t, J=7.3 Hz).

### Example 69

### 1-(3-Chlorobenzyl)-6-(3,5-difluorophenyl)-3-pentanoylaminoindole-2-carboxylic acid

¹H NMR (200 MHz, DMSO-d₆): δ 13.4 (1H, br s), 9.67 (1H, s), 8.08 (1H, s), 7.71 (1H, d, J=8.5 Hz), 7.62-7.51 (3H, m), 7.37-7.18 (3H, m), 7.13-7.09 (1H, m), 7.00-6.92 (1H, m), 5.95 (2H, s), 2.42 (2H, t, J=7.3 Hz), 1.73-1.58 (2H, m), 1.49-1.31 (2H, m), 0.95 (3H, t, J=7.3 Hz).

### Example 70

### 3-[(Biphenyl-4-carbonyl)amino]-1-(3-chlorobenzyl)-6-(3,5-difluorophenyl)indole-2-carboxylic acid

¹H NMR (200 MHz, DMSO-d₆): δ 13.6-12.4 (1H, br s), 10.70 (1H, s), 8.21-8.06 (3H, m), 7.96 (1H, d, J=8.6 Hz), 7.92-7.85 (2H, m), 7.84-7.77 (2H, m), 7.63-7.17 (10H, m), 7.06-6.99 (1H, m), 6.04 (2H, s).

### Example 71

### 1-(3-Chlorobenzyl)-6-(3,5-difluorophenyl)-3-[2-(4-methoxyphenyl)acetylamino]indole-2-carboxylic acid

¹H NMR (200 MHz, DMSO-d₆): δ 9.81 (1H, s), 8.03 (1H, s), 7.63 (1H, d, J=8.6 Hz), 7.59-7.45 (3H, m), 7.35-7.13 (5H, m), 7.09-7.05 (1H, m), 6.95-6.84 (3H, m), 5.91 (2H, s), 3.73 (3H, s), 3.66 (2H, s).

### Example 72

### 1-(3-Chlorobenzyl)-6-(3,5-difluorophenyl)-3-phenylindole-2-carboxylic acid

¹H NMR (200 MHz, DMSO-d₆): δ 13.10 (1H, s), 8.11 (1H, s), 7.60-7.43 (8H, m), 7.42-7.14 (5H, m), 7.03-6.95 (1H, m), 5.96 (2H, s).

### Example 73

### 6-Benzo[1]dioxol-5-yl-1-(3-cyanobenzyl)-3-[4-(dimethylamino)butrylamino]indole-2-carboxylic acid hydrochloride

¹H NMR (200 MHz, DMSO-d₆): δ 13.8-12.8 (1H, br s), 9.89 (1H, s), 7.87-7.84 (1H, m), 7.75-7.67 (2H, m), 7.54 (1H, d, J-8.1 Hz), 7.51-7.39 (2H, m), 7.36 (1H, d, J=1.7 Hz), 7.34-7.28 (1H, m), 7.23 (1H, dd, J-8.2, 1.7 Hz), 7.02 (1H, d, J=8.1 Hz), 6.08 (2H, s), 5.97 (2H, s), 3.19-3.08 (2H, m), 2.79 (6H, s), 2.58-2.48 (2H, m, overlapped with DMSO signal), 2.11-1.94 (2H, m).

### Example 74

### 6-Benzo[1,3]dioxol-5-yl-3-(3-phenylacryloylamino)-1-[3-(2H-tetrazol-5-yl)benzyl]indole-2-carboxylic acid

¹H NMR (200 MHz, DMSO-d₆): δ 9.95 (1H, s), 7.89-7.82 (3H, m), 7.71 (1H, d, J=8.6 Hz), 7.68-7.35 (8H, m), 7.33 (1H, d, J=1.7 Hz), 7.23-7.14 (2H, m), 7.06 (1H, d, J=15.7 Hz), 6.97 (1H, d, J=8.2 Hz), 6.03 (2H, s), 6.01 (2H, s).

### Example 75

### 1-(3-Chlorobenzyl)-5-(4-cyanophenyl)-3-(3,5-dimethoxybenzoylamino)-indole-2-carboxylic acid

¹H NMR (200 MHz, DMSO-d₆): δ 13.8-13.1 (1H, br s), 10.23 (1H, s), 8.08 (1H, s), 7.88 (4H, s), 7.75 (2H, s), 7.38-7.26 (2H, m), 7.24-7.17 (2H, m), 7.16-7.11 (1H, m), 7.05-6.95 (1H, m), 6.76-6.69 (1H, m), 5.90 (2H, s), 3.81 (6H, s).

### Example 76

### 1-(3-Chlorobenzyl)-3-pentanoylamino-6-[4-(trifluoromethyl)phenyl]-indole-2-carboxylic acid

¹H NMR (200 MHz, DMSO-d₆): δ 9.83 (1H, s), 7.96-7.73 (6H, m), 7.47 (1H, d, J=8.7 Hz), 7.33-7.21 (2H, m), 7.10-7.07 (1H, m), 6.97-6.92 (1H, m), 5.93 (2H, s), 2.38 (2H, t, J=7.2 Hz), 1.69-1.55 (2H, m), 1.46-1.28 (2H, m), 0.91 (3H, t, J=7.3 Hz).

### Example 77

### 1-(3-Chlorobenzyl)-3-[(3,3,5,5-tetramethylcyclohexanecarbonyl)amino]-6-[4-(trifluoromethyl)phenyl]-indole-2-carboxylic acid

¹H NMR (200 MHz, DMSO-d₆): δ 13.5-13.2 (1H, br s), 9.68 (1H, s), 8.03-8.00 (1H, m), 7.99-7.94 (2H, m), 7.84-7.80 (2H, m), 7.70 (1H, d, J=8.5 Hz), 7.51 (1H, d, J=8.5 Hz), 7.35-7.25 (2H, m), 7.09 (1H, s), 7.01-6.94 (1H, m), 5.93 (2H, s), 2.85 (1H, t, J=12.4 Hz) 1.63 (2H, d, J=12.4 Hz), 1.34-1.21 (3H, m), 1.12-1.09 (1H, m), 1.06 (6H, s), 0.94 (6H, s).

### Example 78

### 3-Benzoylamino-1-(3-chlorobenzyl)-5-(4-cyanophenyl)indole-2-carboxylic acid

¹H NMR (200 MHz, DMSO-d₆): δ 10.33 (1H, s), 8.13-8.01 (3H, m), 7.88 (4H, s), 7.77-7.73 (2H, m), 7.64-7.49 (3H, m), 7.38-7.26 (2H, m), 7.17-7.12 (1H, m), 7.04-6.96 (1H, m), 5.90 (2H, s).

### Example 79

### 5-(3-Chlorophenyl)-3-phenyl-1-[(3-(trifluoromethoxy)benzyl]indole-2-carboxylic acid

¹H NMR (200 MHz, DMSO-d₆): δ 13.2-13.0 (1H, br s), 7.78-7.70 (1H, m), 7.69-7.62 (3H, m), 7.60-7.30 (9H, m), 7.28-7.14 (2H, m), 7.10-7.02 (1H, m), 5.92 (2H, s).

### Example 80

### 5-(2-Chlorophenyl)-3-phenyl-1-[(3-(trifluoromethoxy)benzyl]indole-2-carboxylic acid

¹H NMR (200 MHz, DMSO-d₆): δ 13.2-13.0 (1H, br s), 7.75-7.67 (1H, m), 7.56-7.19 (14H, m), 7.15-7.07 (1H, m), 5.93 (2H, s).

### Example 81

### 5-(4-Chlorophenyl)-3-phenyl-1-[(3-(trifluoromethoxy)benzyl]indole-2-carboxylic acid

¹H NMR (200 MHz, DMSO-d₆): δ 13.2-13.0 (1H, br s), 7.77-7.56 (5H, m), 7.54-7.30 (8H, m), 7.27-7.13 (2H, m), 7.10-7.02 (1H, m), 5.92 (2H, s).

### Example 82

### 5-(2-Chlorophenyl)-3-pentanoylamino-1-[(3-(trifluoromethoxy)benzyl]-indole-2-carboxylic acid

¹H NMR (200 MHz, DMSO-d₆): δ 13.5-13.2 (1H, br s), 9.63 (1H, s), 7.69-7.58 (2H, m), 7.58-7.50 (1H, m), 7.48-7.31 (5H, m), 7.27-7.17 (1H, m), 7.13 (1H, s), 7.03 (1H, d, f-7.7 Hz), 5.87 (2H, s), 2.36 (2H, t, J=7.3 Hz), 1.67-1.47 (2H, m), 1.44-1.23 (2H, m), 0.88 (3H, t, J=7.2 Hz).

### Example 83

### 5-(3-Chlorophenyl)-3-pentanoylamino-1-[(3-(trifluoromethoxy)benzyl]-indole-2-carboxylic acid

¹H NMR (200 MHz, DMSO-d₆): δ 13.4-13.2 (1H, br s) 9.67 (1H, s) 7.86 (1H, s) 7.74-7.55 (4H, m) 7.53-7.33 (3H, m) 7.27-7.16 (1H, m) 7.09 (1H, s) 6.98 (1H, d, J=7.8 Hz) 5.87 (2H, s) 2.40 (2H, t, J=7.3 Hz) 1.72-1.50 (2H, m) 1.48-1.27 (2H, m) 0.91 (3H, t, J=7.2 Hz).

### Example 84

### 5-(4-Chlorophenyl)-3-pentanoylamino-1-[(3-(trifluoromethoxy)benzyl]-indole-2-carboxylic acid

¹H NMR (200 MHz, DMSO-d₆): δ 13.4-13.1 (1H, br s), 9.65 (1H, s), 7.83 (1H, s), 7.72-7.56 (4H, m), 7.55-7.45 (2H, m), 7.40 (1H, dd, J=8.0 and 8.0 Hz), 7.25-7.16 (1H, m), 7.09 (1H, s), 6.97 (1H, d, J=8.0 Hz), 5.86 (2H, s), 2.40 (2H, t, J=7.2 Hz), 1.70-1.50 (2H, m), 1.48-1.28 (2H, m), 0.91 (3H, t, J=7.2 Hz).

### Example 85

### 1-(3-Chlorobenzyl)-5-(4-chlorophenyl)-3-phenylindole-2-carboxylic acid

¹H NMR (200 MHz, DMSO-d₆): δ 13.2-13.0 (1H, br s), 7.77-7.57 (5H, m), 7.54-7.29 (9H, m), 7.22 (1H, s), 7.05-6.97 (1H, m), 5.87 (2H, s).

### Example 86

### 5-(4-Chlorophenyl)-1-(3,5-dimethylbenzyl)-3-phenylindole-2-carboxylic acid

¹H NMR (200 MHz, DMSO-d₆): δ 13.1-13.0 (1H, br s), 7.73-7.57 (5H, m), δ 7.53-7.30 (7H, m), 6.85 (1H, s), 6.74 (2H, s), 5.77 (2H, s), 2.17 (6H, s).

### Example 87

### 5-(4-Chlorophenyl)-1-(3,5-difluorobenzyl)-3-phenylindole-2-carboxylic acid

¹H NMR (200 MHz, DMSO-d₆): δ 13.2-13.0 (1H, br s), 7.76-7.58 (5H, m), 7.55-7.31 (17H, m), 7.12 (1H, t, J=9.4, 2.5 Hz), 6.86-6.73 (2H, m), 5.89 (2H, s).

### Example 88

### 6-Benzo[1]dioxol-5-yl-3-pentanoylamino-1-(3-phenoxybenzyl)indole-2-carboxylic acid

¹H NMR (200 MHz, DMSO-d₆): δ 13.3 (1H, br s), 9.73 (1H, br s), 7.78 (1H, s), 7.66 (1H, d, J=8.4 Hz), 7.42-7.17 (6H, m), 7.16-7.07 (1H, m), 7.01 (1H, d, J=8.2 Hz), 6.99-6.91 (2H, m), 6.84-6.76 (3H, m), 6.09 (2H, s), 5.93 (2H, s), 2.41 (2H, t, J=7.0 Hz), 1.73-1.56 (2H, m), 1.50-1.30 (2H, m), 0.95 (3H, t, J=7.2 Hz).

### Example 89

### 6-Benzo[1]dioxol-5-yl-1-(9H-fluoren-2-ylmethyl)-3-pentanoylaminoindole-2-carboxylic acid

¹H NMR (200 MHz, DMSO-d₆): δ 13.2 (1H, br s), 10.16 (1H, br s), 7.85-7.75 (4H, m), 7.53 (1H, d, J=7.4 Hz), 7.40-7.26 (5H, m), 7.25-7.13 (2H, m), 7.00 (1H, d, J=8.4 Hz), 6.06 (2H, s), 6.04 (2H, s), 3.83 (2H, s), 2.41 (2H, t, J=7.4 Hz), 1.75-1.58 (2H, m), 1.51-1.30 (2H, m), 0.95 (3H, t, J=7.2 Hz).

### Example 90

### 1-(3-Chlorobenzyl)-6-(3,5-difluorophenyl)-3-(3-phenylacryloylamino)-indole-2-carboxylic acid

¹H NMR (200 MHz, DMSO-d₆): δ 13.52 (1H, br s), 10.1 (1H, s), 8.1.1 (1H, s), 7.79 (1H, d, J=8.5 Hz), 7.74-7.38 (9H, m), 7.36-7.18 (3H, m), 7.16-7.03 (2H, m), 7.01-6.94 (1H, m), 5.98 (2H, s).

### Example 91

### 1-(3-Chlorobenzyl)-6-(3,5-difluorophenyl)-3-(2,2-dimethylpropionylamino)indole-2-carboxylic acid

¹H NMR (200 MHz, DMSO-d₆): δ 13.8-13.2 (1H, s), 9.51 (1H, s), 8.06 (1H, s), 7.81 (1H, d, J=8.6 Hz), 7.63-7.51 (3H, m), 7.37-7.13 (4H, m), 7.01-6.93 (1H, m), 5.96 (2H, s), 1.31 (9H, s).

### Example 92

### 3-(4-Chlorobenzoylamino)-1-(3-chlorobenzyl)-6-(3,5-difluorophenyl)indole-2-carboxylic acid

¹H NMR (200 MHz, DMSO-d₆): δ 10.3 (1H, s), 8.12-8.06 (3H, m), 7.80 (1H, d, J=8.6 Hz), 7.67-7.55 (5H, m), 7.37-7.14 (4H, m), 7.01-6.96 (1H, m), 5.99 (2H, s).

### Example 93

### 1-(3-Chlorobenzyl)-6-(2-isopropoxyphenyl)-3-pentanoylaminoindole-2-carboxylic acid

¹H NMR (200 MHz, DMSO-d₆): δ 13.2-13.5 (1H, br s) 10.4 (1H, br s) 7.85 (1H, d, J=8.5 Hz) 7.56 (1H, s) 7.38-7.24 (4H, m) 7.21 (1H, d, J=8.4 Hz) 7.12-6.96 (4H, m) 5.93 (2H, s) 4.51 (1H, septet, J=6.1 Hz) 2.41 (2H, t, J=7.3 Hz) 1.76-1.58 (2H, m) 1.51-1.31 (2H, m), 1.05 (6H, d, J=6.1 Hz), 0.95 (3H, t, J=7.5 Hz).

### Example 94

### 1-(3-Chlorobenzyl)-6-(2-isopropoxyphenyl)-3-phenylindole-2-carboxylic acid

¹H NMR (200 MHz, DMSO-d₆): δ 12.99 (1H, br s), 7.71 (1H, s), 7.56-7.26 (11H, m), 7.24-7.20 (1H, m), 7.13-6.97 (3H, m), 5.89 (2H, s), 4.54 (1H, septet, J=6.0 Hz), 1.07 (6H, d, J=6.0 Hz).

### Example 95

### 3-[(Biphenyl-4-carbonyl)amino]-1-(3-chlorobenzyl)-6-(2-isopropoxyphenyl)indole-2-carboxylic acid

¹H NMR (200 MHz, DMSO-d₆): δ 11.0-11.3 (1H, br s), 8.22-8.14 (2H, m), 7.97 (1H, d, J=8.4 Hz), 7.93-7.86 (2H, m), 7.84-7.78 (2H, m), 7.65 (1H, s), 7.60-7.44 (3H, m), 7.42-7.24 (5H, m), 7.16-6.98 (4H, m), 5.97 (2H, s), 4.53 (1H, septet, J=6.0 Hz), 1.07 (6H, d, J=6.0 Hz).

### Example 96

### 1-(3-Chlorobenzyl)-6-(4-isopropoxyphenyl)-3-pentanoylaminoindole-2-carboxylic acid

¹H NMR (200 MHz, DMSO-d₆): δ 13.3 (1H, br s), 9.65 (1H, s), 7.81 (1H, s), 7.70-7.92 (3H, m), 7.42 (1H, dd, J=8.5, 1.1 Hz), 7.37-7.25 (2H, m), 7.12-6.94 (4H, m), 5.92 (2H, s), 4.68 (1H, septet, J==6.0 Hz), 2.42 (2H, t, J=7.4 Hz), 1.74-1.56 (2H, m), 1.51-1.29 (2H, m), 1.26 (6H, d, J=6.0 Hz), 0.95 (3H, t, J=7.2 Hz).

### Example 97

### 1-(3-Chlorobenzyl)-6-(4-isopropoxyphenyl)-3-phenylindole-2-carboxylic acid

¹HNMR (200 MHz, DMSO-d₆): δ 12.98 (1H, br s), 7.88 (1H, s), 7.70-7.61 (2H, m), 7.56-7.27 (9H, m), 7.26-7.22 (1H, m), 7.08-6.97 (3H, m), 5.97 (2H, s), 4.68 (1H, septet, J=6.0 Hz), 1.30 (6H, d, 6.0 Hz).

### Example 98

### 1-(3-Chlorobenzyl)-3,6-bis-(4-isopropoxyphenyl)indole-2-carboxylic acid

¹H NMR (200 MHz, DMSO-d₆): δ 12.95 (1H, br s), 7.85 (1H, s), 7.69-7.60 (2H, m), 7.54 (1H, d, J=8.6 Hz), 7.47-7.27 (5H, m), 7.24-7.20 (1H, m), 7.06-6.96 (5H, m), 5.95 (2H, s), 4.69 (1H, septet, J=6.0 Hz), 4.68 (1H, septet, J=6.0 Hz), 1.34 (6H, d, J=6.0 Hz) 1.30 (6H, d, J=6.0 Hz).

### Example 99

### 1-(3-Chlorobenzyl)-6-(3-isopropoxyphenyl)-3-phenylindole-2-carboxylic acid

¹H NMR (200 MHz, DMSO-d₆): δ 13.08 (1H, br s), 7.94 (1H, s), 7.58-7.31 (10H, m), 7.30-7.21 (3H, m), 7.08-7.02 (1H, m), 6.97-6.89 (1H, m), 5.99 (2H, s), 4.73 (1H, septet, J=6.0 Hz), 1.31 (6H, d, J=6.0 Hz).

### Example 100

### 1-(3-Chlorobenzyl)-3-(3-isopropoxyphenyl)-6-(4-isopropoxyphenyl)indole-2-carboxylic acid

¹H NMR (200 MHz, DMSO-d₆): δ 7.81 (1H, s), 7.64-7.57 (2H, m), 7.51 (1H, d, J=8.5 Hz), 7.44-7.38 (1H, m), 7.34 (1H, dd, J=8.0, 2.0 Hz), 7.30-7.24 (2H, m), 7.22-7.19 (1H, m), 7.05-6.93 (5H, m), 6.88 (1H, dd, J=8.0, 2.1 Hz), 5.89 (2H, s), 4.63 (1H, septet, J=6.0 Hz), 4.61 (1H, septet, J=6.0 Hz), 1.28 (6H, d, J=6.0 Hz), 1.26 (6H, d, J=6.0 Hz).

### Example 101

### 1-(3-Chlorobenzyl)-6-(3-isopropoxyphenyl)-3-pentanoylaminoindole-2-carboxylic acid

¹H NMR (200 MHz, DMSO-d₆): δ 13.5-13.1 (1H, br s), 9.64 (1H, br s), 7.84 (1H, s), 7.64 (1H, d, J=8.5 Hz), 7.41 (1H, d, J=8.5 Hz), 7.36 (1H, d, J=7.8 Hz), 7.31-7.17 (4H, m), 7.12-7.08 (1H, m), 6.95-6.85 (2H, m), 5.91 (2H, s), 4.70 (1H, septet, J=6.0 Hz), 2.38 (2H, t, J=7.3 Hz), 1.70-1.53 (2H, m), 1.47-1.27 (2H, m), 1.27 (6H, d, J=6.0 Hz), 0.91 (3H, t, J=7.2 Hz).

### Example 102

### 1-(3-Chlorobenzyl)-6-(4-isopropoxyphenyl)-3-(2-isopropoxyphenyl)indole-2-carboxylic acid

¹H NMR (200 MHz, DMSO-d₆): δ 12.67 (1H, br s), 7.81 (1H, s), 7.67-7.59 (2H, m), 7.43-7.26 (6H, m), 7.19-7.15 (1H, m), 7.13-6.97 (5H, m), 5.96 (2H, s), 4.67 (1H, septet, J=6.0 Hz), 4.46 (1H, septet, J=6.0 Hz), 1.30 (6H, d, J=6.0 Hz), 1.13 (6H, d, J=6.0 Hz).

### Example 103

### 1-(3-Chlorobenzyl)-3-pentanoylamino-5-[4-(trifluoromethyl)phenyl]indole-2-carboxylic acid

¹H NMR (200 MHz, DMSO-d₆): δ 13.5-13.3 (1H, br s), 9.7 (1H, br s), 8.93 (1H, s), 7.90-7.75 (4H, m), 7.73-7.63 (2H, m), 7.35-7.24 (2H, m), 7.13-6.04 (1H, m), 7.00-6.92 (1H, m), 5.84 (2H, s), 2.40 (2H, t, J=7.5 Hz), 1.71-1.51 (2H, m), 1.47-1.26 (2H, m), 0.91 (3H, t, J=7.2 Hz).

### Example 104

### 1-(3-Chlorobenzyl)-5-[4-(methylthio)phenyl]-3-pentanoylaminoindole-2-carboxylic acid

¹H NMR (200 MHz, DMSO-d₆): δ 10.9-10.7 (1H, br s), 8.21 (1H, s), 7.59-7.44 (4H, m), 7.36-7.20 (4H, m), 7.12 (1H, s), 7.06-6.97 (1H, m), 5.96 (2H, s), 2.48 (3H, s, overlapped with DMSO), 2.37 (2H, t, J=7.4 Hz), 1.71-1.51 (2H, m), 1.46-1.24 (2H, m), 0.90 (3H, t, J=7.2 Hz).

### Example 105

### 1-(3-Chlorobenzyl)-3-(4-methoxybenzoylamino)-5-[4-(methylthio)phenyl]-indole-2-carboxylic acid

¹H NMR (200 MHz, DMSO-d₆): δ 12.0-11.8 (1H, br s), 8.47 (1H, s), 8.07-7.96 (2H, m), 7.63-7.47 (4H, m), 7.36-7.22 (4H, m), 7.17 (1H, s), 7.12-7.00 (3H, m), 6.03 (2H, s), 3.80 (3H, s), 2.48 (3H, s, overlapped with DMSO).

### Example 106

### 3-[(Biphenyl-4-carbonyl)amino]-5-(4-tert-butylphenyl)-1-(3-chlorobenzyl)-indole-2-carboxylic acid

¹H NMR (200 MHz, DMSO-d₆): δ 10.4-10.3 (1H, br s), 8.19-8.09 (2H, m), 7.93 (1H, s), 7.89-7.81 (2H, m), 7.80-7.61 (4H, m), 7.62-7.35 (7H, m), 7.34-7.25 (2H, m), 7.18-7.13 (1H, m), 7.05-6.97 (1H, m), 5.88 (2H, s), 1.28 (9H, s).

### Example 107

### 3-(3-Amino-4-methylbenzoylamino)-1-(3-chlorobenzyl)-5-[4-(methylsulfonyl)phenyl]indole-2-carboxylic acid

¹H NMR (200 MHz, DMSO-d₆): δ 10.2-10.1 (1H, br s), 8.16 (1H, s), 8.01-7.86 (4H, m), 7.75-7.72 (2H, m), 7.37-7.24 (3H, m), 7.16-7.11 (2H, m), 7.09-6.93 (2H, m), 5.58 (2H, s), 3.22 (3H, s), 2.11 (3H, s).

### Example 108

### 5-(4-tert-Butylphenyl)-1-(3-chlorobenzyl)-3-(4-isopropoxyphenyl)indole-2-carboxylic acid

¹H NMR (200 MHz, DMSO-d₆): δ 7.64 (1H, s), 7.60-7.34 (8H, m), 7.33-7.22 (3H, m), 7.16-7.06 (1H, m), 6.99-6.89 (2H, m), 5.80 (2H, s), 4.62 (1H, septet, J=6.0 Hz), 1.28 (6H, d, J=6.0 Hz), 1.27 (9H, s).

### Example 109

### 1-(3-Chlorobenzyl)-6-(4-isopropoxyphenyl)-3-(4-methoxybenzoylamino)indole-2-carboxylic acid

¹H NMR (200 MHz, DMSO-d₆): δ 13.5-13.3 (1H, br s), 10.16 (1H, s), 8.09-8.00 (2H, m), 7.86-7.77 (2H, m), 7.70-7.62 (2H, m), 7.42 (1H, d, J=8.5 Hz), 7.38-7.24 (2H, m), 7.16-7.10 (2H, m), 7.07 (1H, s), 7.04-6.96 (3H, m), 5.95 (2H, s), 4.67 (1H, septet, J=6.1 Hz), 3.86 (3H, s), 1.28 (6H, d, J-6.1 Hz).

### Example 110

### N-[1-(3-Chlorobenzyl)-2-hydroxymethyl-6-(4-isopropoxyphenyl)indol-3-yl]-4-methoxybenzamide

LiAlH₄ (6.4 mg, 0.17 mmol) was added to a solution of 1-(3-chlorobenzyl)-6-(4-isopropoxyphenyl)-3-(4-methoxybenzoylamino)indole-2-carboxylic acid ethyl ester, prepared in accordance with the procedure described in Example 7(a), (100 mg, 0.17 mmol) in THF (5 mL) at 0° C. After stirring for 1 h another portion of LiAlH₄ (6.4 mg, 0.17 mmol) was added and the stirring was continued at room temperature for 1 h. The mixture was acidified to pH 2 with HCl (aq., 4M), diluted with water and extracted with EtOAc. The combined extracts were washed with water, brine, and dried over Na₂SO₄ and concentrated. Purification of the residue by chromatography gave the title compound (59 mg, 64%).

¹H NMR (200 MHz, DMSO-d₆): δ 9.85 (1H, s), 8.08-7.99 (2H, m), 7.59 (1H, s), 7.57-7.50 (2H, m), 7.44 (1H, d, J=8.4 Hz), 7.38-7.23 (3H, m), 7.21-7.16 (1H, m), 7.14-7.02 (3H, m), 6.99-6.90 (2H, m), 5.63 (2H, s), 5.20 (1H, t, J=5.1 Hz), 4.62 (1H, septet, J=6.0 Hz), 4.54 (2H, d, J=5.1 Hz), 3.83 (3H, s), 1.25 (6H, d, J=6.0 Hz).

The following Examples 111 to 130 were prepared by analogous techniques to those described herein.

### Example 111

### 5-(4-tert-Butylphenyl)-1-(3-chlorobenzyl)-3-[4-(trifluoromethoxy)phenyl]indole-2-carboxylic acid

¹H NMR (200 MHz, CDCl₃): δ 7.69-7.60 (2H, m), 7.58-7.30 (9H, m), 7.25-7.12 (3H, m), 7.03-6.94 (1H, m), 5.83 (2H, s), 1.34 (9H, s).

### Example 112

### 3-(4-Chlorobenzoylamino)-1-(3-chlorobenzyl)-5-[4-(trifluoromethyl)phenyl]-indole-2-carboxylic acid

¹H NMR (200 MHz, DMSO-d₆): δ 12.4-12.2 (1H, br s), 8.53 (1H, s), 8.10-7.99 (2H, m), 7.92-7.72 (4H, m), 7.64-7.56 (4H, m), 7.34-7.15 (3H, m), 7.10-7.01 (1H, m), 6.04 (2H, s).

### Example 113

### 5-(4-tert-Butylphenyl)-1-(3-chlorobenzyl)-3-[4-(methylthio)phenyl]indole-2-carboxylic acid

¹H NMR (200 MHz, CDCl₃): δ 7.73-7.69 (1H, m), 7.67-7.58 (1H, m), 7.54-7.30 (9H, m), 7.25-7.13 (3H, m), 7.02-6.94 (1H, m), 5.81 (2H, s), 2.56 (3H, s), 1.35 (9H, s).

### Example 114

### 1-(3-Chlorobenzyl)-6-(3,5-difluorophenyl)-3-[4-(dimethylamino)benzoylamino]indole-2-carboxylic acid

¹H NMR (200 MHz, DMSO-d₆): δ 13.6-13.4 (1H, br s), 10.08 (1H, br s), 8.05 (1H, s), 7.95-7.87 (3H, m), 7.59-7.48 (3H, m), 7.31-7.11 (4H, m), 6.97-6.91 (1H, m), 6.82-6.74 (2H, m), 5.96 (2H, br s), 3.00 (6H, s).

### Example 115

### 3-(4-tert-Butylbenzoylamino)-1-(3-chlorobenzyl)-6-(3,5-difluorophenyl)indole-2-carboxylic acid

¹H NMR (200 MHz, DMSO-d₆): δ 13.6-13.4 (1H, br s), 10.24 (1H, s), 8.11 (1H, s), 8.03-7.96 (2H, m), 7.84 (1H, d, J=8.6 Hz), 7.62-7.54 (5H, m), 7.37-7.13 (4H, m), 7.00-6.95 (1H, m), 5.99 (2H, s), 1.34 (9H, s).

### Example 116

### 1-(3-Chlorobenzyl)-6-(3,5-difluorophenyl)-3-(3,5-dimethoxybenzoylamino)indole-2-carboxylic acid

¹H NMR (200 MHz, DMSO-d₆): δ 13.6-13.3 (1H, br s), 10.26 (1H, s), 8.09 (1H, s), 7.82 (1H, d, J=8.6 Hz), 7.60-7.49 (3H, m), 7.31-7.10 (6H, m), 7.00-6.93 (1H, m), 6.73-6.69 (1H, m), 5.97 (2H, s), 3.81 (6H, s).

### Example 117

### 1-(3-Chlorobenzyl)-6-(3,5-difluorophenyl)-3-[2-(3,3,5,5-tetramethylcyclohexyl)acetylamino]-indole-2-carboxylic acid

¹H NMR (200 MHz, DMSO-d₆): δ 13.4-13.3 (1H, br s), 9.68 (1H, s), 8.04 (1H, s), 7.67 (1H, d, J=8.5 Hz), 7.59-7.50 (3H, m), 7.30-7.14 (3H, m), 7.10-7.05 (1H, m), 6.96-6.89 (1H, m), 5.91 (2H, s), 2.25-2.19 (2H, m), 1.55-1.45 (2H, m), 1.25-0.75 (5H, m), 0.99 (6H, s), 0.86 (6H, s).

### Example 118

### 1-(3-Chlorobenzyl)-6-(3,5-difluorophenyl)-3-[2-(3,3,5,5-tetramethylcyclohexyl)acetylamino]indole-2-carboxylic acid sodium salt

¹H NMR (200 MHz, DMSO-d₆): δ 12.09 (1H, s) 7.35 (1H, d, J=8.5 Hz) 7.78 (1H, s) 7.51-7.43 (2H, m) 7.34 (1H, d, J=8.5 Hz) 7.27-7.16 (3H, m) 7.15-7.07 (2H, m) 6.17 (2H, s) 2.22-2.16 (2H, m) 1.54-1.44 (2H, m) 1.27-1.18 (1H, m) 1.06-0.73 (4H, m) 1.00 (6H, s), 0.87 (6H, s).

### Example 119

### 1-(3-Chlorobenzyl)-3-(3-cyclohexylpropionylamino)-6-(3,5-difluorophenyl)indole-2-carboxylic acid

¹H NMR (200 MHz, DMSO-d₆): δ 13.4-13.3 (1H, br s), 9.63 (1H, s), 8.04 (1H, s), 7.66 (1H, d, J=8.6 Hz), 7.58-7.48 (3H, m), 7.30-7.14 (3H, m), 7.08-7.05 (1H, m), 6.95-6.89 (1H, m), 5.91 (2H, s), 2.43-2.34 (2H, m), 1.79-1.48 (7H, m), 1.30-1.10 (4H, m), 0.98-0.81 (2H, m).

### Example 120

### 3-(4-Butylbenzoylamino)-1-(3-chlorobenzyl)-6-(3,5-difluorophenyl)indole-2-carboxylic acid

¹H NMR (200 MHz, DMSO-d₆): δ 13.6-13.3 (1H, br s), 10.21 (1H, s), 8.09 (1H, s), 7.98-7.92 (2H, m), 7.82 (1H, d, J=8.5 Hz), 7.61-7.51 (3H, m), 7.38-7.33 (2H, m), 7.31-7.11 (4H, m), 6.98-6.93 (1H, m), 5.96 (2H, s), 2.69-2.62 (2H, m), 1.66-1.51 (2H, m), 1.40-1.22 (2H, m), 0.89 (3H, t, J=7.2 Hz).

### Example 121

### 1-(3-Chlorobenzyl)-6-(3,5-difluorophenyl)-3-(4-isopropylbenzoylamino)indole-2-carboxylic acid

¹H NMR (200 MHz, DMSO-d₆): δ 13.6-13.3 (1H, br s), 10.26 (1H, br s), 8.08 (1H, m), 8.00-7.94 (2H, m), 7.83 (1H, d, J=8.6 Hz), 7.60-7.50 (3H, m), 7.43-7.37 (2H, m), 7.32-7.11 (4H, m), 7.00-6.93 (1H, m), 5.97 (2H, s), 2.98 (1H, septet, J=7.4 Hz), 1.24 (6H, d, J=7.4 Hz).

### Example 122

### 3-[(1-Adamantylcarbonyl)amino]-1-(3-chlorobenzyl)-6-(3,5-difluorophenyl)indole-2-carboxylic acid

¹H NMR (200 MHz, DMSO-d₆): δ 13.8-13.2 (1H, br s), 9.45 (1H, s), 8.03 (1H, s), 7.79 (1H, d, J=8.6 Hz), 7.58-7.46 (3H, m), 7.32-7.08 (4H, m), 6.96-6.88 (1H, m), 5.92 (2H, s), 2.04-2.00 (3H, m), 1.96-1.95 (6H, m), 1.72-1.69 (6H, m).

### Example 123

### 3-[(1-Adamantylcarbonyl)amino]-1-(3-chlorobenzyl)-6-(3,5-difluorophenyl)indole-2-carboxylic acid sodium salt

¹H NMR (200 MHz, DMSO-d₆): δ 12.34 (1H, s), 8.37 (1H, d, J=8.6 Hz), 7.74 (1H, s), 7.48-7.41 (2H, m), 7.31-7.16 (4H, m), 7.14-7.04 (2H, m), 6.16 (2H, s), 2.05-1.99 (3H, m), 1.96-1.91 (6H, m), 1.73-1.68 (6H, m).

### Example 124

### 1-(3-Chlorobenzyl)-6-(3,5-difluorophenyl)-3-[4-(trifluoromethoxy)benzoylamino]indole-2-carboxylic acid

¹H NMR (200 MHz, DMSO-d₆): δ 13.6-13.3 (1H, br s), 10.38 (1H, s), 8.19-8.09 (3H, m), 7.78 (1H, d, J=8.6 Hz), 7.58-7.52 (5H, m), 7.32-7.11 (4H, m), 6.99-6.94 (1H, m), 5.97 (2H, s).

### Example 125

### 1-(3-Chlorobenzyl)-3-(4-cyanobenzoylamino)-6-(3,5-difluorophenyl)indole-2-carboxylic acid

¹H NMR (200 MHz, DMSO-d₆): δ 13.5-13.3 (1H, br s), 10.50 (1H, s), 8.22-8.15 (2H, m), 8.12-8.01 (3H, m), 7.77 (1H, d, J=8.6 Hz), 7.60-7.51 (3H, m), 7.35-7.10 (4H, m), 7.00-6.94 (1H, m), 5.97 (2H, s).

### Example 126

### 6-(4-Butylphenyl)-1-(3-chlorobenzyl)-3-[(3,5-dimethyladamant-1-ylcarbonyl)amino]indole-2-carboxylic acid

¹H NMR (200 MHz, DMSO-d₆): δ 13.5-13.4 (1H, br s), 9.51 (1H, s), 7.81 (1H, s), 7.77 (1H, d, J=8.8 Hz), 7.66-7.58 (2H, m), 7.42-7.36 (1H, m), 7.29-7.23 (4H, m), 7.10-7.09 (1H, m), 6.96-6.91 (1H, m), 5.90 (2H, s), 2.60 (2H, t, J=7.2 Hz), 2.15-2.08 (1H, m), 1.81-1.78 (2H, m), 1.62-1.48 (6H, m), 1.40-1.24 (6H, m), 1.19-1.16 (2H, m) 0.89 (3H, t, J=7.2 Hz), 0.85 (6H, s).

### Example 127

### 6-(4-Butylphenyl)-1-(3-chlorobenzyl)-3-[(3,5-dimethyladamant-1-ylcarbonyl)amino]indole-2-carboxylic acid sodium salt

¹H NMR (200 MHz, DMSO-d₆): δ 12.30 (1H, s), 8.30 (1H, d, J=8.5 Hz), 7.57-7.50 (3H, m), 7.26-7.15 (6H, m), 7.11-7.05 (1H, m), 6.13 (2H, s), 2.57 (2H, t, J=7.2 Hz), 2.15-2.08 (1H, m), 1.79-1.74 (2H, m), 1.59-1.47 (6H, m), 1.39-1.25 (6H, m), 1.19-1.15 (2H, m) 0.88 (3H, t, J=7.2 Hz), 0.85 (6H, s).

### Example 128

### 6-(4-Butylphenyl)-1-(3-chlorobenzyl)-3-[4-(2,5-dimethylpyrrol-1-yl)benzoylamino]indole-2-carboxylic acid

¹H NMR (200 MHz, DMSO-d₆): δ 13.4-13.2 (1H, br s), 10.48 (1H, br s), 8.20-8.12 (2H, m), 7.88 (1H, s), 7.81 (1H, d, J=8.5 Hz), 7.69-7.61 (2H, m), 7.48-7.42 (3H, m), 7.32-7.24 (4H, m), 7.16-7.13 (1H, m), 7.04-6.98 (1H, m), 5.96 (2H, br s), 5.83 (2H, s), 2.60 (2H t, J=7.2 Hz), 2.01 (6H, s), 1.63-1.49 (2H, m), 1.39-1.22 (2H, m), 0.89 (3H, t, J=7.2 Hz).

### Example 129

### 6-(4-Butylphenyl)-1-(3-chlorobenzyl)-3-[4-(trifluoromethoxy)benzoylamino]indole-2-carboxylic acid

¹H NMR (200 MHz, DMSO-d₆): δ 13.5-13.3 (1H, br s), 10.37 (1H, s), 8.20-8.14 (2H, m), 7.88 (1H, s), 7.76 (1H, d, J=7.6 Hz), 7.66-7.60 (2H, m), 7.59-7.53 (2H, m), 7.47-7.41 (1H, m), 7.32-7.23 (4H, m), 7.15-7.11 (1H, m), 7.02-6.96 (1H, m), 5.95 (2H, s), 2.60 (2H, t, J=7.2 Hz), 1.63-1.49 (2H, m), 1.39-1.21 (2H, m), 0.89 (3H, t, J=7.2 Hz).

### Example 130

### 6-(4-Butylphenyl)-1-(3-chlorobenzyl)-3-[4-(trifluoromethoxy)benzoylamino]indole-2-carboxylic acid sodium salt

¹H NMR (200 MHz, DMSO-d₆): δ 13.67 (1H, s), 8.51 (1H, d, J=7.6 Hz), 8.18-8.12 (2H, m), 7.61-7.51 (5H, m), 7.31-7.18 (6H, m), 7.14-7.08 (1H, m), 6.19 (2H, s), 2.57 (2H, t, J=7.2 Hz), 1.62-1.47 (2H, m), 1.35-1.21 (2H, m), 0.88 (3H, t, J=7.2 Hz).

### Example 131

### 6-(4-Butylphenyl)-1-(3-chlorobenzyl)-3-(4-isopropoxybenzoylamino)indole-2-carboxylic acid

### (a) 6-(4-Butylphenyl)-1-(3-chlorobenzyl)-3-(4-isopropoxybenzoylamino)indole-2-carboxylic acid ethyl ester

A mixture of 3-amino-6-(4-butylphenyl)-1-(3-chlorobenzyl)indole-2-carboxylic acid ethyl ester (250 mg, 540 nmol), 4-isopropoxybenzoyl chloride (162 mg, 810 nmol), DMAP (33 mg, 270 nmol), triethylamine (229 μL, 1.63 mmol) and dry MeCN (2 mL) was stirred at room temperature under argon for 18 h and then heated at 80° C. for 10 min, at 100° C. for 5 min and finally at 12⁰° C. for 10 min using microwave irradiation. The mixture was poured into HCl (1M) and extracted with EOAc. The combined extracts were washed with NaHCO₃, dried with Na₂SO₄ and concentrated. The residue was crystallised from EOAc/benzene to yield the title compound (150 mg, 44%).

### (b) 6-(4-Butylphenyl)-1-(3-chlorobenzyl)-3-(4-isopropoxybenzoylamino)indole-2-carboxylic acid

The title compound (45 mg, 32%) was prepared by hydrolysis of 6-(4-butylphenyl)-1-(3-chlorobenzyl)-3-(4-isopropoxybenzoylamino)indole-2carboxylic acid ethyl ester (see Example 131(a)) under conditions as hereinbefore described, for example at 90° C. for 10 min in 1,4-dioxane.

¹H NMR (200 MHz, DMSO-d₆): δ 13.5-13.3 (1H, br s), 10.14 (1H, s), 8.03-7.95 (2H, m), 7.86 (1H, s), 7.80 (1H, d, J=7.6 Hz), 7.67-7.60 (2H, m), 7.46-7.39 (1H, m), 7.31-7.23 (4H, m), 7.14-7.11 (1H, m), 7.08-7.04 (2H, m), 7.01-6.95 (1H, m), 5.94 (2H, s), 4.74 (1H, septet, J=6.0 Hz), 2.60 (2H, t, J=7.2 Hz), 1.64-1.49 (2H, m), 1.36-1.25 (2H, m), 1.30 (6H, d, J=6.0 Hz), 0.89 (3H, t, J=7.2 Hz).

### Example 132

### 6-(4-Butylphenyl)-1-(3-chlorobenzyl)-3-(3-isopropoxybenzoylamino)indole-2-carboxylic acid

The title compound was prepared in accordance with the procedures described herein.

¹H NMR (200 MHz, DMSO-d₆): δ 13.5-13.3 (1H, br s), 10.25 (1H, s), 7.87 (1H, s), 7.75 (1H, d, J=8.5 Hz), 7.67-7.54 (4H, m), 7.47-7.37 (2H, m), 7.34-7.22 (4H, m), 7.16-7.10 (2H, m), 7.02-6.95 (1H, m), 5.94 (2H, s), 4.71 (1H, septet, J=6.0 Hz), 2.60 (2H, t, J=7.2 Hz), 2.15-2.08 (2H, m), 1.36-1.21 (2H, m), 1.29 (6H, d, J=6.0 Hz), 0.88 (3H, t, J=7.2 Hz).

### Example 133

### 6-(4-Butylphenyl)-1-(3-chlorobenzyl)-3-(3-isopropoxybenzoylamino)indole-2-carboxylic acid sodium salt

The title compound was prepared in accordance with the procedures described herein.

¹H NMR (200 MHz, DMSO-d₆): δ 13.58 (1H, s) 8.52 (1H, d, J=8.6 Hz) 7.60-7.51 (5H, m) 7.46-7.38 (1H, m) 7.29-7.17 (6H, m) 7.14-7.07 (2H, m) 6.18 (2H, s) 4.69 (1H, septet, J=6.0 Hz) 2.58 (2H, t, J=7.2 Hz) 1.63-1.49 (2H, m) 1.35-1.24 (2H, m) 1.30 (6H, d, J=6.0 Hz), 0.88 (3H, t, J=7.2 Hz).

### Example 134

### 6-(4-Carboxyphenyl)-3-(4-chlorobenzoylamino)-1-(3-chlorobenzyl)indole-2-carboxylic acid

The title compound was prepared in accordance with the procedures described herein.

¹H NMR (200 MHz, DMSO-d₆): δ 13.6-12.8 (2H, br s), 10.37 (1H, s), 8.13-7.99 (5H, m), 7.94-7.86 (2H, m), 7.82 (1H, d, J=8.6 Hz), 7.68-7.61 (2H, m), 7.59-7.51 (1H, m), 7.38-7.24 (2H, m), 7.17-7.11 (1H, m), 7.04-6.95 (1H, m), 5.99 (2H, s).

### Example 135

### 3-(4-Chlorobenzoylamino)-1-(3-chlorobenzyl)-6-(4-hydroxymethylphenyl)indole-2-carboxylic acid

### (a) 3-(4-Chlorobenzoylamino)-1-(3-chlorobenzyl)-6-(4-hydroxymethylphenyl)indole-2-carboxylic acid ethyl ester

1M BH₃×THF (1M, 200 μL, 0.20 mmol) was added to a stirred solution of 6-(4-carboxyphenyl)-3-(4-chlorobenzoylamino)-1-(3-chlorobenzyl)indole-2-carboxylic acid ethyl ester, prepared by analogous techniques to those described hereinbefore, (120 mg, 0.20 mmol) in THF (5 mL) at −5° C. The temperature of the mixture was allowed to reach room temperature and stirring was continued for 12 h whereafter another portion of 1M BH₃×THF (1M, 200 μL, 0.20 mmol) was added. The mixture was stirred for 8 h at room temperature and poured into AcOH (aq., 50%, 20 mL) and stirred for 1 h. The mixture was extracted with EtOAc and the combined extracts were washed with H₂O and brine, dried over Na₂SO₄ and concentrated. Purification of the residue by chromatography gave the sub-title compound (90 mg, 78%).

### (b) 3-(4-Chlorobenzoylamino)-1-(3-chlorobenzyl)-6-(4-hydroxymethylphenyl)indole-2-carboxylic acid

The title compound was prepared by hydrolysis of 3-(4-chlorobenzoylamino)-1-(3-chlorobenzyl)-6-(4-hydroxymethylphenyl)indole-2-carboxylic acid ethyl ester in accordance with the procedure in Example 2(b) (NaOH (1M), MeCN, 80° C., 20 min).

¹H NMR (200 MHz, DMSO-d₆): δ 13.5-13.2 (1H, br s), 10.35 (1H, s), 8.13-8.04 (2H, m), 7.92 (1H, s), 7.79 (1H, d, J=8.4 Hz), 7.70-7.57 (4H, m), 7.51-7.25 (5H, m), 7.17-7.12 (1H, m), 7.04-6.96 (1H, m), 5.98 (2H, s), 5.14-5.34 (1H, m), 4.62-4.54 (2H, m).

### Example 136

### 6-(4-Butylphenyl)-1-(3-chlorobenzyl)-3-[3-(2,5-dimethylpyrrol-1-yl)benzoylamino]indole-2-carboxylic acid

### (a) 6-(4-Butylphenyl)-1-(3-chlorobenzyl)-3-(3-nitrobenzamido)indole-2-carboxylic acid ethyl ester

The sub-title compound was prepared in accordance with Example 131(a) using 3-nitrobenzoyl chloride (room temperature overnight, and then 90° C. for 10 min, 110° C. for 10 min and finally 130° C. for 20 min).

### (b) 3-(3-Aminobenzamido)-6-(4-butylphenyl)-1-(3-chlorobenzyl)indole-2-carboxylic acid ethyl ester

6-(4-Butylphenyl)-1-(3-chlorobenzyl)-3-(3-nitrobenzamido)indole-2-carboxylic acid ethyl ester (235 mg, 0.39 mmol; see Example 136(a)) was hydrogenated at ambient pressure and temperature using palladium on charcoal (10%, 62 mg) in EtOAc for 6 h, then filtered through Celite®. The filtrate was concentrated and the residue was crystallised from EtOAc/benzene to yield the sub-title compound (140 mg, 62%).

### (c) 6-(4-Butylphenyl)-1-(3-chlorobenzyl)-3-(3-(2,5-dimethylpyrrol-1-yl)benzamido)indole-2-carboxylic acid ethyl ester

A mixture of 3-(3-aminobenzamido)-6-(4-butylphenyl)-1-(3-chlorobenzyl)indole-2-carboxylic acid ethyl ester (140 mg, 240 nmol; see Example 136(b)), hexane-2,5-dione (189 μL, 1.21 mmol), p-toluenesulfonic acid (50 ng, 0.24 nmol) and toluene (1 mL) was stirred at room temperature for 1 h and then heated at 70° C. for 20 min using microwave irradiation. The mixture was diluted with EOAc, washed with Na₂CO₃ (aq., sat.), dried over Na₂SO₄, and concentrated. The residue was crystallised from petroleum ether/benzene to yield the sub-title compound (97 mg, 61%).

### (d) 6-(4-Butylphenyl)-1-(3-chlorobenzyl)-3-[3-(2,5-dimethylpyrrol-1-yl)benzoylamino]indole-2-carboxylic acid

The title compound was prepared by hydrolysis of 6-(4-butylphenyl)-1-(3-chlorobenzyl)-3-[3-(2,5-dimethylpyrrol-1-yl)benzoylamino]indole-2-carboxylic acid ethyl ester (see Example 136(c)) in accordance with the procedure described in Example 2(b) (2M KOH, dioxane, 70° C., 10 min, then 80° C. 20 min and finally 90° C. 10 min).

¹H NMR (200 MHz, DMSO-d₆): δ 13.5-13.3 (1H, br s), 10.8-10.4 (1H, br s), 8.14-8.08 (1H, m), 7.93-7.80 (3H, m), 7.73-7.69 (1H, m), 7.66-7.60 (2H, m), 7.55-7.49 (1H, m), 7.43 (1H, d, J=8.5 Hz), 7.31-7.23 (4H, m), 7.13-7.11 (1H, m), 7.02-6.96 (1H, m), 5.96 (2H, s), 5.83 (2H, s), 2.59 (2H, t, J=7.2 Hz), 2.00 (6H, s), 1.63-1.49 (2H, m), 1.39-1.21 (2H, m), 0.88 (3H, t, J=7.2 Hz).

### Example 137

### 6-(4-Butylphenyl)-1-(3-chlorobenzyl)-3-[3-(2,5-dimethylpyrrol-1-yl)benzoylamino]indole-2-carboxylic acid sodium salt

The title compound was prepared in accordance with the procedures described herein.

¹H NMR (200 MHz, DMSO-d₆): δ 13.91 (1H, br s), 8.57 (1H, d, J=8.5 Hz), 8.11-8.07 (1H, m), 7.83-7.81 (1H, m), 7.74-7.66 (1H, m), 7.61-7.54 (3H, m), 7.52-7.76 (1H, m), 7.31-7.19 (6H, m), 7.14-7.09 (1H, m), 6.19 (2H, s), 5.84 (2H, s), 2.59 (2H, t, J=7.2 Hz), 2.03 (6H, s), 1.64-1.49 (2H, m), 1.38-1.22 (2H, m), 0.89 (3H, t, J=7.2 Hz).

### Example 138

### 3-(4-Chlorobenzoylamino)-1-(4-chlorobenzyl)-5-[4-(trifluoromethyl)phenyl]-indole-2-carboxylic acid

The title compound was prepared in accordance with the procedures described herein.

¹H NMR (200 MHz, DMSO-d₆): δ 13.5-13.1 (1H, br s), 10.6-10.4 (1H, br s), 8.12-8.00 (3H, m), 7.93-7.84 (2H, m), 7.82-7.68 (4H, m), 7.68-7.57 (2H, m), 7.41-7.30 (2H, m), 7.13-7.03 (2H, m), 5.89 (2H, m).

### Example 139

### 3-[Acetyl-(4-methoxybenzyl)amino]-1-(3-chlorobenzyl)-5-(4-isopropoxyphenyl)indole-2-carboxylic acid

### (a) 3-Bromo-5-(4-isopropoxyphenyl)indole-2-carboxylic acid ethyl ester

A solution of NBS (0.904 5.082 mmol) in acetone (10 mL) was added dropwise to a solution of 5-(4-isopropoxyphenyl)indole-2-carboxylic acid ethyl ester, prepared in accordance with Example 1(a) from 5-bromoindolecarboxylic acid ethyl ester and 4-isopropoxyphenylboronic acid, (1.5 g, 4.62 mmol) in acetone (35 mL) at room temperature. After 2.5 h an additional portion of NBS (164 mg, 0.92 mmol) was added and temperature of the mixture was increased to 45° C. After 1.5 h the mixture was cooled to room temperature, poured into Na₂S₂O₃ (aq., 10%) and extracted with EtOAc. The extract was washed with Na₂S₂O₃ (aq., 10%), NaHCO₃ (aq., sat) and brine, dried over Na₂SO₄ and concentrated. The residue was crystallised from EtOH to give the sub-title compound (1.63 g, 88%).

### (b) 1-(3-Chlorobenzyl)-5-(4-isopropoxyphenyl)indole-2-carboxylic acid ethyl ester

The sub-title compound was prepared in accordance with Example 1(c) from 3-bromo-5-(4-isopropoxyphenyl)indole-2-carboxylic acid ethyl ester (1.0 g, 2.5 mmol, see step (a) above) and 3-chlorobenzylchloride (0.604 g, 3.75 mmol). Yield 0.784 g (60%).

### (c) 1-(3-Chlorobenzyl)-5-(4-isopropoxyphenyl)-3-[(4-methoxybenzyl)amino]indole-2-carboxylic acid ethyl ester

4-Methoxybenzylamine (60 μL, 0.46 mmol) was added to a mixture of 1-(3-chlorobenzyl)-5-(4-isopropoxyphenyl)indole-2-carboxylic acid ethyl ester (200 mg, 0.38 mmol), Pd₂(dba)₃ (17.4 mg, 0.019 mmol), BINAP (35.5 mg, 0.057 mmol), Cs₂CO₃ (173 mg, 0.53 mmol) and anhydrous toluene (10 mL). The mixture was stirred at 120° C. for 24 h, cooled to room temperature and diluted with Et₂O. The mixture was filtered through Celite® and the filter cake washed with Et₂O. The combined filtrates were concentrated and the residue purified by chromatography to yield the sub-title compound (0.213 g, 96%).

### (d) 3-[Acetyl-(4-methoxybenzyl)amino]-1-(3-chlorobenzyl)-5-(4-isopropoxyphenyl)indole-2-carboxylic acid ethyl ester

Acetyl chloride (24 μL, 0.34 mmol) was added at room temperature to a solution of 1-(3-chlorobenzyl)-5-(4-isopropoxyphenyl)-3-[(4-methoxybenzyl)amino]indole-2-carboxylic acid ethyl ester (200 mg, 0.34 mmol) in dry toluene (2 mL). The mixture was stirred at 70° C. for 1 h, allowed to cool to room temperature, diluted with EtOAc and washed with NaHCO₃ (aq., sat.), brine and finally dried over Na₂SO₄. Concentration and purification by chromatography gave the sub-title compound (0.136 g, 64%).

### (e) 3-[Acetyl-(4-methoxybenzyl)amino]-1-(3-chlorobenzyl)-5-(4-isopropoxyphenyl)indole-2-carboxylic acid

The title compound was prepared by hydrolysis of 3-[acetyl-(4-methoxybenzyl)amino]-1-(3-chlorobenzyl)-5-(4-isopropoxyphenyl)indole-2-carboxylic acid ethyl ester (136 mg, 0.217 mmol; see step (d) above), in accordance with the procedure in Example 1(e) (NaOH (aq.), dioxane, 80° C., 1 h). Yield: 93 mg (72%).

¹H NMR (200 MHz, DMSO-d₆): δ 13.7-13.3 (1H, br s), 7.55-7.40 (2H, m), 7.35-7.24 (4H, m), 7.15-7.06 (2H, m), 7.02-6.84 (5H, m), 6.80-6.71 (2H, m), 5.86 (2H, s), 5.34 (1H, d, J=14.0 Hz), 4.61 (1H, septet J=6.0 Hz), 4.21 (1H, d, J=14.0 Hz), 3.64 (3H, s), 1.72 (3H, s), 1.26 (6H, d, J=6.0 Hz).

### Example 140

### 1-(3-Chlorobenzyl)-3-{(4-chlorobutyryl)-[2-(4-fluorophenyl)ethyl]amino}-5-(4-isopropoxyphenyl)indole-2-carboxylic acid

The title compound was prepared in accordance with the procedure in Example 139(c)-(e) from 1-(3-chlorobenzyl)-5-(4-isopropoxyphenyl)indole-2-carboxylic acid ethyl ester, 2-(4-fluorophenyl)ethylamine and 4-chlorobutyryl chloride.

¹H NMR (200 MHz, DMSO-d₆): δ 7.54-7.36 (5H, m), 7.34-6.87 (10H, m), 6.05 (1H, d, J=16.0 Hz), 5.87 (1H, d, J=16.0 Hz), 4.60 (1H, septet, J=6.0 Hz), 4.19-3.96 (1H, m), 3.82-3.61 (1H, m), 3.22 (2H, t, J=6.4 Hz), 2.85-2.70 (2H, m), 2.37-2.10 (1H, m), 2.07-1.85 (1H, m), 1.67-1.44 (2H, m), 1.25 (6H, d, J=6.0 Hz).

### Example 141

### 1-(3-Chlorobenzyl)-3-{(6-chloropyridine-3-carbonyl)-[2-(4-fluorophenyl)ethyl]amino}-5-(4-isopropoxyphenyl)indole-2-carboxylic acid

The title compound was prepared in accordance with the procedure in Example 139(c)-(e) from 1-(3-chlorobenzyl)-5-(4-isopropoxyphenyl)indole-2-carboxylic acid ethyl ester, 2-(4-fluorophenyl)ethylamine and 6-chloronicotinoyl chloride.

¹H NMR (200 MHz, DMSO-d₆): δ 8.31 (1H, d, J=1.9 Hz), 7.82 (1H, s), 7.75 (1H, dd, J=8.2, 1.9 Hz), 7.66-7.43 (4H, m), 7.30 (1H, d, J=8.2 Hz), 7.21-6.91 (9H, m), 6.38-6.29 (1H, m), 6.06 (1H, d, J=16.6 Hz), 5.60 (1H, d, J=16.6 Hz), 4.63 (1H, septet, J=6.1 Hz), 4.11-3.93 (2H, m), 3.09-2.75 (2H, m), 1.27 (6H, d, J=6.1 Hz).

### Example 142

Title compounds of the invention were tested in the biological test described above and were found to exhibit 50% inhibition of mPGES-1 at a concentration of 10M or below. For example the inhibition of mPGES-1 is exemplified by the following compounds of the examples, as listed in the following table:

Example 1: 96% inhibition at 10M

Example 2: 100% inhibition at 10M

Example 3: 85% inhibition at 10M

Example 4: 81% inhibition at 10M

Example 5: 93% inhibition at 10M

Example 6: 100% inhibition at 10M

1. A compound of formula I, 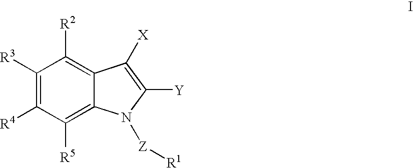
wherein X represents an optionally substituted aryl or heteroaryl group or an optionally substituted amide, amine or sulfonamide group, which latter three groups are connected to the indole ring through their nitrogen atom;
Y represents a carboxylic acid, a carboxylic acid ester, a carboxylic acid amide, a hydroxamic acid, a hydroxamic acid ester or hydroxymethyl;
Z represents a spacer group;
R¹ represents an optionally substituted aryl or heteroaryl group;
one of the groups R², R³, R⁴ and R⁵ represents an optionally substituted aryl or heteroatyl group.

2. A compound as claimed in claim 1, wherein;
X represents:
i) an aryl group or a heteroaryl group, both of which groups are optionally substituted by one or more substituents selected from A; or
ii) —N(R⁶)-E-R⁷;
E represents a single bond, —C(O)— or —S(O)ₙ—;
Y represents —CH₂OH, —C(O)N(H)R⁸, —C(O)N(H)OR⁸ or —C(O)OR⁸;
Z represents a C₁₋₈ alkylene or a C₂₋₈ heteroalkylene chain, both of which:
(i) optionally contain one or more unsaturations;
(ii) are optionally substituted by one or more substituents selected from halo, —R⁸, —N(R⁸)(R⁹), —OR⁸ and ═O; and/or
(iii) may form part of an additional 3- to 8-membered ring formed between any one or more members of the C₁₋₈ alkylene or C₂₋₈ heteroalkylene chain, which ring optionally contains 1 to 3 heteroatoms and/or 1 to 3 unsaturations and which ring is itself optionally substituted by one or more substituents selected from halo, —R⁸, —N(R⁸)(R⁹), —OR⁸ and ═O;
R¹ represents an aryl or a heteroaryl group, both of which are optionally substituted by one or more substituents selected from A;
one of the groups R², R³, R⁴ and R⁵ represents an aryl group or a heteroaryl group (both of which are optionally substituted by one or more substituents selected from A) and:
a) the other groups are independently selected from hydrogen, G¹, an aryl group, a heteroaryl group (which latter two groups are optionally substituted by one or more substituents selected from A), C₁₋₆ alkyl, C₃₋₁₀ cycloalkyl, C₂₋₆ alkenyl. C₂₋₆ alkynyl or C₃₋₈ heterocycloalkyl (which latter five groups are optionally substituted by one or more substituents selected from G¹ and/or Q¹); and/or
b) any two other groups which are adjacent to each other are optionally linked to form, along with two atoms of the essential benzene ring in the compound of formula I, a 5- to 6-membered ring, optionally containing 1 or more heteroatoms and/or 1 to 3 unsaturations, which ring is itself optionally substituted by one or more substituents selected from halo, —R⁸, —OR⁸ and ═O;
A represents, on each occasion when mentioned above:
I) an aryl group or a heteroaryl group, both of which are optionally substituted by one or more substituents selected from B;
II) a C₁₋₆ alkyl, C₃₋₁₀ cycloalkyl, C₂₋₆ alkenyl, C₂₋₆ alkynyl or C₃₋₈ heterocycloalkyl group, all of which are optionally substituted by one or more substituents selected from G¹ and/or Q¹; or
III) a G¹ group; or
IV) two adjacent A substituents may be linked together to form, along with the essential atoms of the aryl or heteroaryl group to which the two A substituents are attached, a further 5- to 6-membered ring, optionally containing 1 or more heteroatoms and/or 1 to 3 unsaturations, which ring is itself optionally substituted by one or more substituents selected from halo, —R⁸, —OR⁸ and ═O;
G¹ represents, on each occasion when mentioned above, halo, cyano, —N₃, —NO₂, —ONO₂ or -A¹-R¹⁰;
wherein A¹ represents a single bond or a spacer group selected from —C(Q²)A²-, —S(O)ₙA³-, —N(R¹¹)A⁴-, —OA⁵- and —S—, in which:
A² represents A⁶ or —S—;
A³ represents A⁶;
A⁴ represents A⁷, —C(Q²)N(R¹¹)C(Q²)N(R¹¹)—, —C(Q²)N(R¹¹)C(Q²)O—,
—C(Q²)N(R¹¹)S(O)ₙN(R¹¹)—, C(Q²)S—, —S(O)ₙN(R¹¹)C(Q²)N(R¹¹)—,
—S(O)ₙN(R¹¹)C(Q²)O—, —S(O)ₙN(R¹¹)S(O)ₙN(R¹¹)— or —S(O)ₙO—;
A⁵ represents A⁷ or —S(O)ₙO—;
A⁶ represents a single bond, —N(R¹¹)— or O—;
A⁷ represents a single bond, —C(Q²)-, —C(Q²)N(R¹¹)—, —C(Q²)O—, —S(O)ₙ— or —S(O)ₙN(R¹¹);
Q¹ and Q² independently represent, on each occasion when mentioned above, ═O, ═S, ═NR¹⁰, ═NN(R¹⁰)(R¹¹), ═NOR¹⁰, ═NS(O)₂N(R¹⁰)(R¹¹), ═NCN, ═C(H)NO₂ or ═C(R¹⁰)(R¹¹);
R⁶ and R⁷ independently represent, on each occasion when mentioned above:
I) hydrogen;
II) an aryl group or a heteroaryl group, both of which are optionally substituted by one or more substituents selected from B; or
III) a C₁₋₆ alkyl, C₃₋₁₀ cycloalkyl, C₂₋₆ alkenyl, C₂₋₆ alkynyl or C₃₋₈ heterocycloalkyl group, all of which groups are optionally substituted by one or more substituents selected from G² and/or Q³; or
R⁶ and R⁷ may be linked together to form along with the N atom and -E- group to which R⁶ and R⁷ are respectively attached, a 5- to 8-membered ring, optionally containing 1 to 3 heteroatoms and/or 1 to 3 unsaturations, which ring is optionally substituted by one or more substituents selected from G² and/or Q³;
B represents, on each occasion when mentioned above:
I) an aryl group or a heteroaryl group, both of which are optionally substituted by one or more substituents selected from G² and/or wherein any two adjacent atoms of the aryl or heteroaryl group may be linked together to form a further 5- to 6-membered ring, optionally containing 1 or more heteroatoms and/or 1 to 3 unsaturations, which ring is itself optionally substituted by one or more-substituents selected from halo, —R⁸, —OR⁸ and ═O;
II) a C₁₋₆ alkyl, C₃₋₁₀ cycloalkyl, C₂₋₆ alkenyl, C₂₋₆ alkynyl or C₃₋₈ heterocycloalkyl group, all of which are optionally substituted by one or more substiuents selected from G² and/or Q³; or
III) a G² group; or
IV) two adjacent B substituents may be linked together to form, along with the essential atoms of the aryl or heteroaryl group to which the two B substituents are attached, a further 5- to 6-membered ring, optionally containing 1 or more heteroatoms and/or 1 to 3 unsaturations, which ring is itself optionally substituted by one or more substituents selected from halo, —R⁸, —OR⁸ and ═O;
G² represents, on each occasion when mentioned above, halo, cyano, —N₃, —NO₂, —ONO₂ or -A⁸-R¹²;
wherein A³ represents a single bond or a spacer group selected from —C(Q⁴)A⁹-, —S(O)ₙA¹⁰-, —N(R¹³)A¹¹-, —OA¹²- and —S—, in which:
A⁹ represents A¹³ or —S—;
A¹⁰ represents A¹³;
A¹¹ represents A¹⁴, —C(Q⁴)N(R¹³)C(Q⁴)N(R¹³)—, —C(Q⁴)N(R¹³)C(Q⁴)O—, —C(Q⁴)N(R¹³)S(O)ₙN(R¹³)—, —C(Q⁴)S—, —S(O)ₙN(R¹³)C(Q⁴)N(R¹³)—, —S(O)N(R¹³)C(Q⁴)O—, —S(O)ₙN(R¹³)S(O)N(R¹³)— or —S(O)ₙO—;
A¹² represents A¹⁴ or —S(O)ₙO—;
A¹³ represents a single bond, —N(R¹³)— or —O—;
A¹⁴ represents a single bond, —C(Q⁴)-, —C(Q⁴)N(R¹³)—, —C(Q⁴)O—, —S(O)ₙ— or —S(O)ₙN(R¹³);
Q³ and Q⁴ independently represent, on each occasion when mentioned above, ═O, ═S, ═NR¹², ═NN(R¹²)(R¹³), ═NOR¹², ═NS(O)₂N(R¹²)(R¹³), ═NCN, ═C(H)NO₂ or ═C(R¹²)(R¹³);
R⁸, R⁹, R¹⁰, R¹¹, R¹² and R¹³ are independently selected from:
i) hydrogen;
ii) an aryl or a heteroaryl group, both of which are optionally substituted by one or more substituents selected from G³ and/or wherein any two adjacent atoms of the aryl or heteroaryl group may be linked together to form a further 5- to 6-membered ring, optionally containing 1 or more heteroatoms, which ring is itself optionally substituted by one or more substituents selected from halo, —R¹⁴, —OR¹⁴ and ═O; or
iii) a C₁₋₆ alkyl, C₃₋₁₀ cycloalkyl, C₂₋₆ alkenyl, C₂₋₆ alkynyl or C₃₋₈ heterocycloalkyl group, all of which are optionally substituted by one or more substituents selected from G³ and/or W¹; or
any pair of R⁸, R⁹, R¹⁰, R¹¹, R¹² and R¹³ may, for example when present on the same or on adjacent atoms, be linked together to form with those, or other relevant, atoms, a further 5- to 8-membered ring, optionally containing 1 to 3 heteroatoms and/or 1 to 3 unsaturations, which ring is itself optionally substituted by one or more substituents selected from G³ and/or W¹;
G³ represents, on each occasion when mentioned above, halo, cyano, —N₃, —NO₂, —ONO₂ or -A¹⁵-R¹⁵;
wherein A¹⁵ represents a single bond or a spacer group selected from —C(W²)A¹⁶-, —S(O)ₙA¹⁷-, —N(R¹⁶)A¹⁸-, —OA¹⁹- and —S—, in which:
A¹⁶ represents A²⁰ or —S—;
A¹⁷ represents A²⁰;
A¹⁸ represents A²¹, —C(W²)N(R¹⁶)C(W²)N(R⁶)_, —C(W²)N(R¹⁶)C(W²)O—, —C(W²)N(R¹⁶)S(O)ₙN(R¹⁶)—, —C(W²)S—, —S(O)ₙN(R¹⁶)C(W²)N(R¹⁶)_, —S(O)ₙN(R¹⁶)C(W²)O—, —S(O)ₙN(R¹⁶)S(O)ₙN(R¹⁶)— or —S(O)ₙO—;
A¹⁹ represents A²¹ or —S(O)ₙO—;
A²⁰ represents a single bond, —N(R¹⁶)— or —O—;
A²¹ represents a single bond, —C(W²)—, —C(W²)N(R¹⁶)—, —C(W²)O—, —S(O)ₙ— or —S(O)ₙN(R¹⁶);
W¹ and W² independently represent, on each occasion when mentioned above, ═O, ═S, ═NR¹⁵, ═NN(R¹⁵)(R¹⁶), ═NOR¹⁵, ═NS(O)₂N(R¹⁵)(R¹⁶), ═NCN, ═C(H)NO₂ or ═C(R¹⁵)(R¹⁶);
R¹⁴, R¹⁵ and R¹⁶ are independently selected from:
i) hydrogen;
ii) an aryl or a heteroaryl group, both of which are optionally substituted by one or more substituents selected from G⁴, methylenedioxy, difluoromethylenedioxy and/or dimethylmethylenedioxy; or
iii) a C₁₋₆ alkyl, C₃₋₁₀ cycloalkyl, C₂₋₆ alkenyl, C₂₋₆ alkynyl or C₃₋₈ heterocycloalkyl group, all of which are optionally substituted by one or more substituents selected from G⁴ and/or J; or
any pair of R¹⁴, R¹⁵ and R¹⁶ may, for example when present on the same or on adjacent atoms, be linked together to form with those, or other relevant, atoms, a further 5- to 7-membered ring, optionally containing 1 to 3 heteroatoms and/or 1 to 3 unsaturations, which ring is itself optionally substituted by one or more substituents selected from G⁴ and J;
G⁴ represents, on each occasion when mentioned above, halo, cyano, —N₃, —NO₂, —ONO₂ or -A²²-R¹⁷;
wherein A²² represents a single bond or a spacer group selected from —C(O)A²³—, —S(O)A²⁴-N(R¹⁸)A²⁵-, —OA²⁶- and —S—, in which:
A²³ represents A²⁷ or —S—;
A²⁴ represents A²⁷;
A²⁵ represents A²⁸, —C(O)N(R¹⁸)C(O)N(R¹⁸)—, —C(O)N(R¹⁸)C(O)O—, —C(O)N(R¹⁸)S(O)ₙN(R¹⁸)—, —C(O)S—, —S(O)ₙN(R¹⁸)C(O)N(R¹⁸)—, —S(O)ₙN(R¹⁸)C(O)O—, —S(O)ₙN(R¹⁸)S(O)ₙN(R¹⁸)— or —S(O)ₙO—;
A²⁶ represents A²⁸ or —S(O)ₙO—;
A²⁷ represents a single bond, —N(R¹⁸)— or —O—;
A²⁸ represents a single bond, —C(O)—, —C(O)N(R¹⁸)—, —C(O)O—, —S(O)ₙ— or —S(O)ₙN(R¹⁸);
J represents, on each occasion when mentioned above, ═O, ═S, ═NR¹⁷, ═NN(R⁷)(R¹⁸), ═NOR¹⁷, ═NS(O)₂N(R⁷)(R¹⁸), ═NCN, ═C(H)NO₂ or ═C(R¹⁷)(R¹⁸);
R¹⁷ and R¹⁸ are independently selected from hydrogen and C₁₋₆alkyl, which latter group is optionally substituted by one or more substituents selected from halo, —NH₂, —N(H)Me, —N(H)Et, —N(H)i-Pr, —NMe₂, —N(Me)Et, —N(Me)i-Pr, —NEt₂, —OH, —OMe, —OEt, -Oi-Pr and ═O; and
n represents, on each occasion when mentioned above, 1 or 2,
or a pharmaceutically-acceptable salt thereof.

3. A compound as claimed in claim 2, wherein n represents 2.

4. A compound as claimed in claim 2, wherein A represents G¹ or any two adjacent A substituents may be linked by a methylenedioxy group.

5. A compound as claimed in claim 2, wherein G¹ represents halo, cyano, —NO₂ or -A¹-R¹⁰.

6. A compound as claimed in claim 2, wherein A² represents A⁶.

7. A compound as claimed in claim 2, wherein A³ and A⁵ independently represent a single bond.

8. A compound as claimed in claim 2, wherein A⁴ represents a single bond, —C(Q²)- or —S(O)₂—.

9. A compound as claimed in claim 2, wherein Q² represents ═O.

10. A compound as claimed in claim 2, wherein B represents G².

11. A compound as claimed in claim 2, wherein G² represents halo, cyano, —NO₂— or -A⁸-R¹².

12. A compound as claimed in claim 2, wherein A⁸ represents a single bond, —N(R³)A¹¹- or —OA¹²-.

13. A compound as claimed in claim 2, wherein A¹¹ and A¹² independently represent a single bond.

14. A compound as claimed in claim 1, wherein Z represents C₁₋₆ alkylene, in which one of the carbon atoms in the chain may be replaced with oxygen.

15. A compound as claimed in claim 1, wherein Y represents —CH₂OH, —C(O)NHR⁸ or —C(O)OR⁸.

16. A compound as claimed in claim 1, wherein R¹ represents optionally substituted fluorenyl, phenyl or pyridyl.

17. A compound as claimed in claim 1, wherein (when X represents an optionally substituted aryl or heteroaryl group) X represents an optionally substituted phenyl, thienyl, pyridyl, pyrazolyl, pyrazinyl or quinolinyl group.

18. A compound as claimed in any one of the preceding claim 1, wherein (when they represent an optionally substituted aryl or heteroaryl group) R², R³, R⁴ and R⁵ represent optionally substituted phenyl, pyridyl or naphthyl.

19. A compound as claimed in claim 18, wherein the other substituents on the benzene ring of the indole represent hydrogen or G¹.

20. A compound as claimed in claim 2, wherein R⁶ represents hydrogen or C₁₋₃ alkyl group (which latter group is optionally substituted by G²).

21. A compound as claimed in claim 2, wherein R⁷ represents phenyl or pyridyl (which groups are optionally substituted by one or more substituents selected from B), or C₁₋₄ alkyl, C₂₋₄ alkenyl or C₅₋₁₀ cycloalkyl (which latter three groups are optionally substituted by one or more substituents selected from G²).

22. A compound as claimed in claim 2, wherein R⁶ and R⁷ are linked to form a 5- to 6-membered ring optionally substituted by ═O.

23. A compound as claimed in any one of claims 2 to 22, wherein R⁸ and R¹³ independently represent C₁₋₃ alkyl or hydrogen.

24. A compound as claimed in claim 2, wherein R¹⁰ represents hydrogen, phenyl, tetrazolyl, C₁₋₄ alkyl, C₂₋₄ alkenyl or C₅₋₆ cycloalkyl, which latter five groups are optionally substituted by one or more substituents selected from G³.

25. A compound as claimed in claim 2, wherein R¹² represents hydrogen, phenyl, pyrrolyl, C₁₋₄alkyl or C₅₋₁₀ cycloalkyl, which latter four groups are optionally substituted by one or more substituents selected from G³.

26. A compound as claimed in claim 2, wherein R¹¹ represents hydrogen or C₂₋₄ alkenyl.

27. A compound as claimed in claim 2, wherein G³ represents halo, —R¹⁵ or —OR⁵.

28. A compound as claimed in claim 2, wherein R¹⁵ represents hydrogen, C₁₋₃ alkyl or phenyl.

29. A compound as claimed in claim 16, wherein the optional substituents are selected from halo, —NO₂, cyano, methylenedioxy, C₁₋₆ alkyl (which alkyl group is optionally substituted by one or more substituents selected from a halo group, a phenyl group and OR¹⁹), C₂₋₆ alkenyl, C₃₋₁₀ cycloalkyl (which cycloalkyl group is optionally substituted with C₁₋₆ alkyl), phenyl (which group is optionally substituted with one or more substituents selected from halo and OR¹⁹), a heteroaryl group selected from tetrazolyl and pyrrolyl (which groups are optionally substituted by one or more C₁₋₆ alkyl groups), methylthio, methylsulfonyl, methylsulfonyl, ═O, —OR¹⁹, —N(R¹⁹)R²⁰, —C(O)OR¹⁹, —C(O)R¹⁹, —C(O)N(R¹⁹)R²⁰, —S(O)₂N(R¹⁹)R²⁰ and/or —N(R¹⁹)S(O)₂R², wherein R¹⁹ and R²⁰ independently represent hydrogen, phenyl, C₁₋₄ alkenyl, C₁₋₆ alkyl (which alkyl group is optionally substituted by one or more fluoro atoms) or a phenyl group and R²¹ represents phenyl or C₁₋₆ alkyl (which alkyl group is optionally substituted by one or more fluoro atoms).

30. A compound as defined in claim 1, or a pharmaceutically-acceptable salt thereof, for use as a pharmaceutical.

31. A pharmaceutical formulation including a compound as defined in claim 1, or a pharmaceutically-acceptable salt thereof, in admixture with a pharmaceutically acceptable adjuvant, diluent or carrier.

32. The use of a compound as defined in claim 1, or a pharmaceutically-acceptable salt thereof, for the manufacture of a medicament for the treatment of a disease in which inhibition of the activity of microsomal prostaglandin E synthase-1 is desired and/or required.

33. A use as claimed in claim 32, wherein the disease is inflammation.

34. A use as claimed in claim 33 wherein the disease is inflammatory bowel disease, irritable bowel syndrome, migraine, headache, low back pain, fibromyalgia, a myofascial disorder, a viral infection, a bacterial infection, a fungal infection, dysmenorrhea, a burn, a surgical or dental procedure, a malignancy, atherosclerosis, gout, arthritis, osteoarthritis, juvenile arthritis, rheumatoid arthritis, rheumatic fever, ankylosing spondylitis, systemic lupus erythematosus, vasculitis, pancreatitis, nephritis, bursitis, conjunctivitis, iritis, scleritis, uveitis, wound healing, dermatitis, eczema, psoriasis, stroke, diabetes, a neurodegenerative disorder, an autoimmune disease, osteoporosis, asthma, chronic obstructive pulmonary disease, pulmonary fibrosis, an allergic disorder, rhinitis, an ulcer, coronary heart disease, sarcoidosis or any other disease with an inflammatory component.

35. A method of treatment of a disease in which inhibition of the activity of mPGES-1 is desired and/or required, which method comprises administration of a therapeutically effective amount of a compound as defined in claim 1, or a pharmaceutically-acceptable salt thereof, to a patient suffering from, or susceptible to, such a condition.

36. A combination product comprising:
(A) a compound as defined in claim 1, or a pharmaceutically-acceptable salt thereof, and
(B) another therapeutic agent that is useful in the treatment of inflammation, wherein each of components (A) and (B) is formulated in admixture with a pharmaceutically-acceptable adjuvant, diluent or carrier.

37. A combination product as claimed in claim 36 which comprises a pharmaceutical formulation including a compound as defined above, or a pharmaceutically-acceptable salt thereof, another therapeutic agent that is useful in the treatment of inflammation, and a pharmaceutically-acceptable adjuvant, diluent or carrier.

38. A combination product as claimed in claim 36 which comprises a kit of parts comprising components:
(a) a pharmaceutical formulation including a compound as defined above, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

39. A process for the preparation of a compound as defined in claim 2, which comprises:
(i) reaction of a compound of formula II, 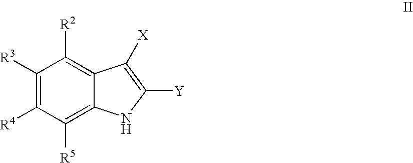
wherein X Y, R², R³, R⁴ and R⁵ are as defined in claim 2, with a compound of formula II,
R¹ZL¹ III
wherein L¹ represents a suitable leaving group and R¹ and Z are as defined in claim 2;
(ii) reaction of a compound of formula IV, 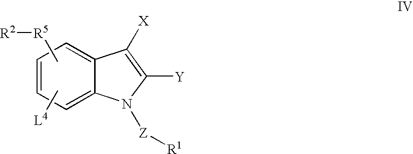
wherein L⁴ represents L² or L³, in which L² and L³ represent appropriate leaving groups and L⁴ is attached to one or more of the carbon atoms of the benzenoid ring of the indole, and the remaining positions of the benzenoid ring are substituted with 1 to 3 (depending on the number of L⁴ substituents) substituents R² to R⁵ as appropriate, and Z, X, Y, R¹, R², R³, R⁴ and R⁵ are as defined in claim 2, with a compound of formula V,
R²²L⁵ V
wherein R²² represents R², R³, R⁴ or R⁵ (as appropriate), and L⁵ represents L² (when L⁴ is L³) or L³ (when L⁴ is L²) as defined above;
(iii) for compounds of formula I in which X represents an optionally substituted aryl or heteroaryl group, reaction of a compound of formula VI, 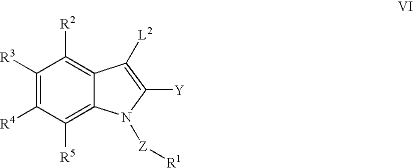
wherein L² is as defined above and Z, Y, R¹, R², R³, R⁴ and R⁵ are as defined in claim 2, with a compound of formula VII,
X^{a}L³ VII
wherein L³ is as defined above and Xa represents an aryl or heteroaryl group, optionally substituted as defined in claim 2;
(iv) for compounds of formula I in which X represents
—N(R⁶)-E-R⁷,
reaction of a compound of formula VI as defined above, with a compound of formula VIII,
HN(R⁶)-E-R⁷
wherein E, R⁶ and R⁷ are as defined in claim 2;
(v) for compounds of formula I in which X represents —N(R⁶)-E-R⁷, reaction of a compound of formula IX, 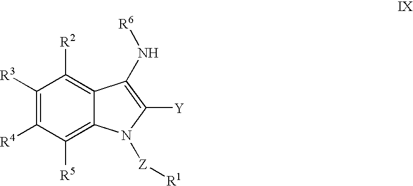
wherein Z, Y, R, R², R³, R⁴, R⁵ and R⁶ are as defined in claim 2, with a compound of formula X,
R⁷-E-L X
wherein L¹ is as defined above and E and R⁷ are as defined in claim 2;
(vi) for compounds of formula I in which E represents a single bond and R⁷ is a C₁₋₆ alkyl group, C₃₋₆ alkenyl or a C₃₋₆ alkynyl group, reduction of a compound of formula I, wherein X represents —C(O)— and R⁷ represents H, a C₁₋₅, alkyl group, a C₂₋₅ alkenyl or a C₂₋₅ alkynyl group.
